(19) United States
(12) Patent Application Publication
(10) Pub. No.: US 2026/0049291 A1
(43) Pub. Date: Feb. 19, 2026

(54) CHIMERIC POXVIRUSES

(71) Applicant: Transgene, Illkirch Graffenstaden (FR)

(72) Inventors: Philippe ERBS, Illkirch Graffenstaden (FR); Annie FINDELI, Illkirch Graffenstaden (FR)

(73) Assignee: Transgene, Illkirch Graffenstaden (FR)

(21) Appl. No.: 19/102,438

(22) PCT Filed: Aug. 18, 2023
(86) PCT No.: PCT/EP2023/072773

§ 371 Date: Feb. 10, 2025

(30) Foreign Application Priority Data
Aug. 18, 2022 (EP) 22306234.0

(51) Int. Cl. C12N 7/00; A61K 35/768; A61P 35/00

(52) U.S. Cl. CPC C12N 7/00; A61K 35/768; A61P 35/00; C12N 2710/24021; C12N 2710/24033; C12N 2710/24052

(57) ABSTRACT

The present invention relates to a chimeric poxvirus with improved anticancer activity (higher cancer cell killing capacities and better tumor selectivity), as well as variant version thereof with one or more altered viral genes, recombinant versions thereof comprise one or more nucleic acid(s) of interest and recombinant variant versions thereof, all of which may be included in a composition and used for the treatment of a disease, in particular proliferative disease, notably cancers.

### TECHNICAL FIELD OF THE INVENTION

The present invention is in the field of oncolytic viruses and provides new chimeric poxviruses particularly useful for treating proliferative diseases like, but not limited to, cancers. More precisely, the invention provides chimeric poxviruses which are obtained by the pooling of different strains of parental poxviruses and the selection of more potent chimeric viruses by cellular passages. These chimeric poxviruses present many improved characteristics compared to their parental Vaccinia virus strain Copenhagen (COP) which is known in the art as being a particularly effective oncolytic viral vector, and more specifically compared to their parental poxviruses.

The chimeric poxviruses according to the invention may be modified by altering the thymidine kinase-encoding gene (locus J2R) and/or the ribonucleotide reductase-encoding gene (locus|4L and/or F4L), forming variant chimeric poxviruses.

The chimeric poxviruses according to the invention may further encode for one or more heterologous transgene(s), forming recombinant chimeric poxviruses. Said recombinant chimeric poxviruses also express higher rates of transgenes compared to their parental poxviruses.

The present invention also deals with methods for obtaining chimeric poxviruses.

The chimeric poxviruses of the invention may be used for treatment of proliferative diseases, like cancers.

### BACKGROUND ART

Oncolytic viruses are a class of therapeutic agents that have the unique property of tumor-dependent self-perpetuation (Hermiston et al., 2006, Curr. Opin. Mol. Ther., 8 (4): 322-30). The benefit of using these viruses is that as they replicate, they lyse their host cells. Oncolytic viruses are capable of selective replication in dividing cells (mainly cancer cells) while leaving non-dividing cells (e.g.: healthy cells or primary cells) unharmed. As the infected dividing cells are destroyed by lysis, they release new infectious particles to infect the surrounding dividing cells. Due to its potential to be more effective and less toxic than current therapies due to the viruses' selective growth and amplification in tumor cells, oncolytic virus therapy has been recognized as a promising therapeutic approach for cancer treatment. Cancer cells are ideal hosts for many viruses because they have the antiviral interferon pathway inactivated or have mutated tumor suppressor genes that enable viral replication to proceed unhindered (Chernajovsky et al., 2006, BMJ, 332 (7534): 170-2). Several viruses including adenovirus, herpes simplex virus, reovirus, poxvirus, Newcastle disease virus, measles virus, Vesicular Stomatitis Virus, Seneca Valley Virus and the hemagglutinating virus of Japan Envelope have been clinically tested as oncolytic agents.

Among them, oncolytic poxviruses demonstrated encouraging results in multiple pre-clinical tumor models and some clinical trials for the treatment of various cancers. Six poxviruses from four genera have been investigated as potential oncolytic viruses: Vaccinia virus, Raccoonpox virus and Cowpoxvirus (Orthopoxviruses), Myxoma virus (Leporipoxvirus), Yaba monkey tumor virus (Yatapoxvirus) and Squirrelpox virus (Torres-Domingez et al., 2019, Review Expert Opin Biol Ther.; 19 (6): 561-573). Poxviruses represent about 13% of the total number of clinical studies evaluating oncolytic viruses from 2000 and 2020 (Macedo et al., 2020, Journal for ImmunoTherapy of Cancer; 8). Among them, recombinant oncolytic Vaccinia viruses (VACV) are promising vectors for tumor therapy. The genome organization, lysis capacity and wide tumor tropism of VACV make it an ideal oncolytic agent for cancer treatment and the most used poxvirus vector for cancer therapy (Haddad et al., 2017, Front Oncol., 7, 96. doi: 10.3389/fonc.2017.00096). It targets tumors selectively after systemic administration and thus displays natural tumor tropism (McFadden, 2005, Nat Rev Microbiol., 3, 201-213). Several strains of VACV are currently evaluated in preclinical and clinical trials including Wyeth, Western Reserve, Copenhagen and Lister strains (Heo et al., 2013 Nat Med., 19, 329-336. doi: 10.1038/nm.3089, Zeh et al., 2015, Mol Ther., 23, 202-214. doi: 10.1038/mt.2014.194, Foloppe et al., 2008, Gene Ther., 15, 1361-1371. doi: 10.1038/gt.2008.82, Mell et al., 2017, Clin Cancer Res., 23, 5696-5702. doi: 10.1158/1078-0432.CCR-16-3232). However, neither rabbitpox viruses nor cowpox viruses have been evaluated in clinical studies, for different reasons. Rabbitpox viruses are too virulent to be used as oncolytic viruses for treating human cancers, which may be explained by the presence of three specific virulence genes, encoding a zinc RING finger protein, an ankyrin repeat family protein and a chemokine-binding protein (Li et al., 2005, Journal of General Virology, 86, 2969-2977). Cowpox viruses, despite interesting results in vitro (Ricordel et al. 2017, Mol. Ther. Oncolytics Vol. 7), have a too weak oncolytic power in vivo to represent a potential oncolytic virus platform.

Modifications of the naturally occurring viruses have already been practiced in order to enhance the ability of poxviruses, and more particularly of vaccinia viruses, to infect and lyse 100% of the tumor cells, which is difficult to achieve in in vivo context. For this purpose, many strategies are currently used to modify the viruses (e.g.: tropism modification to redirect virus to the cancer cell surface). Oncolytic vaccinia viruses are often “armed” with enzyme-prodrug systems that enhance the oncolytic efficacy of the virus therapy by exerting a strong bystander effect and thus permit elimination of neighboring uninfected tumor cells. For example, armament with the so-called FCU1 suicide gene, encoding a bifunctional chimeric polypeptide that combines the enzymatic activities of FCY1 and FUR1, efficiently catalyzed the direct conversion of 5-fluorocytosine (5-FC), a nontoxic antifungal agent, into the toxic metabolites 5-fluorouracil (5-FU) and 5-fluorouridine-5′monophosphate (5-FUMP), thus bypassing the natural resistance of certain human tumor cells to 5-fluorouracil (Erbs et al., 2000, Cancer Res., 60 (14): 3813-22). Foloppe et al. showed that a VACV expressing the FCU1 gene has potent anti-tumor effect both in vitro and in vivo in a murine model of a human colon tumor (Foloppe et al., 2008, Gene Ther., 15:1361-1371). The vaccinia viruses expressing the FCU1 fusion suicide gene, combined with the administration of the 5-fluorocytosine (5-FC) prodrug, displayed a highly potent anti-tumor effect both in vitro and in vivo. The potential of FCU1 delivered by an oncolytic cowpox virus as therapeutic agent was also explored (Ricordel et al., 2017, Molecular Therapy-Oncolytics, 7:1-11). The cowpox viruses expressing the FCU1 fusion suicide gene, combined with the administration of the prodrug, also displayed an anti-tumor effect in vitro.

Viral modifications can also be used to increase safety. In this regard, thymidine kinase (TK) deleted poxvirus was shown to have decreased pathogenicity compared with wild-type poxvirus, but replication in tumor cells was preserved (Buller et al., 1985, Nature, 317 (6040): 813-5). Attenuated poxviruses, and in particular vaccinia virus strains, have thus been developed for therapeutic and diagnostic applications and are being evaluated in clinical studies. However, methods of attenuating vaccinia viruses lead to a decrease of their efficacy. In a therapeutic point of view, this decrease of efficacy can result into a diminution of overall response, a diminution of patient's survival, an increase of mortality, pathology resistances . . . As a result, the first oncolytic vaccinia viruses tested in clinical trials have been highly safe in patients but have generally fallen short of their expected therapeutic value as monotherapies. Moreover, some tumor cells appeared to be poorly permissive or resistant to infection and replication of oncolytic poxviruses, meaning that some cancers are still refractory or resistant to oncolytic poxvirus-based therapy (e.g. National Clinical Trial NCT01380600: safety but no efficacy of Pexa-Vec (pexastimogene devacirepvec, JX-594, an oncolytic and immunotherapeutic vaccinia Wyeth (WY) based virus engineered to express GM-CSF) in colorectal carcinoma patients who are refractory to, or intolerant to, oxaliplatin, irinotecan, and Erbitux treatments; National Clinical Trial NCT01387555: no efficacy of Pexa-Vec in patients suffering from advanced liver cancer who failed sorafenib; National Clinical Trial NCT01636284: no efficacy of Pexa-Vec in sorafenib-naïve advanced liver cancer patients).

Another way to obtain evolved viruses is the generation of new oncolytic chimeric viruses via a directed evolution method, a method used to mimic and accelerate the process of natural selection.

Viruses undergo genetic changes by several mechanisms, including point mutation and recombination. Recombination is a widespread phenomenon in viruses and can have a major impact on their evolution. Recombination occurs when at least two viral homologous sequences co-infect the same host cells and exchange genetic fragments. Homologous recombination (HR) occurs in the same site in both parental strands and creates new genetic combinations that may change the phenotype of the chimeric viruses. Homologous recombination is the basis for many widely used genetic techniques in virus research, including construction of recombinant vectors (Hruby, 1990, Clin. Microbiol. Rev, 3 (2) 153-170). In 1958, experimentations showed that different strains of poxviruses could recombine (Fenner and Comben. 1958, Virology, 5, 530-548). Some characteristics of the obtained chimeras were studied (e.g.: virus replication, heat resistance or hemagglutinin production), but no oncolytic power or therapeutic index were explored. Paszkowski et al. have studied the mechanism of poxvirus genetic recombination (Paszkowski et al., 2016, PLOS Pathogens, 12 (8) e1005824). They observed multiple genetic exchanges even after one round of selection, showing that homologous intramolecular and intermolecular recombination occurred efficiently; however, no functional characteristics were studied.

The directed evolution methodology is usually employed for the creation of gene libraries (Koerber et al., 2006, Nat. Protocols 1 (2) p.701-706). This methodology applied to oncolytic virotherapy has a different purpose. It was recently used for obtaining oncolytic chimeric poxviruses, CF33 and CF17, by the pooling of nine strains of poxviruses known to be oncolytic on non-resistant tumor cells (WO2018/031694, O'Leary et al., 2018, Mol. Therap. Vol 9:13-21, Chaurasiya et al., 2020, Cancer Gene Therapy 27:125-135, Hammad et al., 2020, Mol. Ther. Oncolytics, vol. 19 p. 278-282). The mix of viruses was grown and shuffled on a non-tumor cell line (African green monkey kidney fibroblasts CV-1), a cell line usually used in the research field for poxvirus production, due to its high permissiveness to poxviruses replication. However, in infected HCT116 colorectal cancer cell line, the generated chimeric CF33 virus secreted less extra cellular enveloped viruses (EEV) at early stages than IHD parental strain, reflecting a lower spreading capacity of the CF33 virus in tumor cells, compared to at least one of the parental strains. Moreover, the overall viral titer of CF33 in the HCT116 cell lysates was found to be similar to the one obtained with parental Western Reserve (WR) and Elstree strains 72 h post-infection, showing that CF33 virus did not replicate more than at least two parental viruses. Moreover, O'Leary et al. does not provide any data on the effect of the chimera on healthy cells (preferably primary cells). The tumoral specificity of the chimeras is thus unknown and cannot be compared to the tumoral specificity of the parental strains.

DeVV5, an oncolytic chimeric vaccinia virus, was also generated by a directed evolution method (WO2020011754, Ricordel et al., 2018 Cancers, July 10; 10 (7): 231). Four different vaccinia virus strains (Modified Vaccinia Virus Ankara (MVA), Copenhagen (COP), Wyeth (WY) and Western Reserve (WR)) were pooled in resistant cancer cell lines, amplified, and selected through successive passages under stringent conditions. However, even if the selected chimeric vaccinia virus deVV5 showed enhanced oncolytic properties and tumor selectivity compared to its parental viruses in vitro, but no results were shown in vivo.

Combination therapies with standards and emerging anticancer therapies (chemotherapy, immune-checkpoint inhibitors (ICI), etc.) are also used to improve the oncolytic potency of oncolytic poxviruses (Filley et al., 2017, Front Oncol. 7, 106. doi: 10.3389/fonc.2017.00106). For example, Pexa-Vec was evaluated in a randomized controlled Phase 3 trial in advanced first line hepatocarcinoma (HCC) comparing the administration of Pexa-Vec and sorafenib to sorafenib alone (National Clinical Trial NCT02562755). TG6002 (Heinrich et al., 2017, Onco Targets Ther., 10, 2389-2401. doi: 10.2147/OTT.S126320), which is a derivative of a Copenhagen (COP) based TK-deleted VACV expressing the FCU1 fusion suicide gene and given in combination with 5-FC, entered clinical development in recurrent glioblastoma patients (National Clinical Trial NCT03294486). BT-001 (Vaccinia Virus encoding GM-CSF and aCTLA4) is tested in a phase I/IIa study, combined with Pembrolizumab (a-PD1) in patients with cutaneous or subcutaneous lesions or easily injectable lymph nodes of metastatic/advanced solid tumors (National Clinical Trial NCT04725331). The trial evaluating the combination of Pexa-Vec with Nivolumab for the treatment of HCC in patients naive to sorafenib was prematurely terminated, due to the failure of Pexa-Vec and nivolumab in their respective pivotal trials (National Clinical Trial NCT03071094).

### Technical Problem and Proposed Solution

Despite the different current approaches tested, like viral modifications, viral armaments, chimeras' generation or combinations with standards or emerging therapies, the known poxvirus platforms do not have a sufficient oncolytic power to provide satisfying therapeutic results for the treatment of cancers. Moreover, poxviruses are not capable of infecting and replicating in all tumor cells, which means that some cancers are refractory or resistant to therapies based on oncolytic poxviruses. Furthermore, oncolytic poxviruses can be limited in their intratumoral spread because of physical barriers within the tumor microenvironment; neutralizing antibodies can also impede the poxviruses' systemic delivery.

As a result, there is still a need for highly potent oncolytic poxviruses, with improved abilities to infect and lyse many tumor cells without increasing the injection dose, to avoid toxic events. The use of these poxviruses with better anti-cancer efficacy will result in better cancer cell killing capacities compared to the currently developed oncolytic viruses, whether they are administrated in monotherapy or combined with other anticancer therapies. The use of these poxviruses will also result in an increased oncolytic effect on cells poorly permissive or resistant to current therapies with existing oncolytic viruses, oncolytic poxviruses or oncolytic vaccinia viruses.

Moreover, there is a need of safe oncolytic viruses: the poxviruses should have improved tumor selectivity to be of safe use for treated subjects, but with no or no significant impact on the virus' cancer cell killing capacities.

There is also a need of oncolytic poxviruses being less neutralized by the host immune system, to avoid the reduction of their effects because of rapid systemic elimination.

In the context of the invention, the inventors showed that it was possible to create and select new chimeric poxviruses and recombinant versions thereof, which importantly and surprisingly achieved better anticancer therapy, with an increased oncolytic power, an increased extracellular-enveloped virus (EEV)-secretion capacity, and a better spreading capacity, resulting in higher cancer cell killing capacities, a better antitumor efficacy both in injected tumors (tumors into which the viruses are directly injected) and non-injected tumors (tumors in which viruses are not injected, said viruses being administered through another route, for example intravenous, subcutaneous, etc.) and increased survival of subjects receiving the chimeric poxviruses. In the situation where these chimeric poxviruses carry a transgene encoding a protein of interest, their metabolism, and more particularly their rapid replication and their ability to infect a high number of cells, due to the chimeras' increased capacity to produce EEV, lead to an increased ability to produce proteins of interest. By inducing also, the syncytia formation, chimeric poxviruses according to the invention enhance virus yield, the said spreading and cytopathic effect, as well as antitumor immunity. Chimeric poxviruses according to the invention also replicate less in healthy cells (preferably primary cells), having thus a better tumor selectivity and safety profile. Having both a better oncolytic power and a better safety profile, said chimeric poxviruses have an increased therapeutic index. They are also less neutralized by anti-vaccinia virus antibodies, thus allowing their use in subjects carrying such antibodies (e.g.: subjects vaccinated with vaccinia viruses and therefore having been induced an adaptive immune response) and more resistant to complement-mediated virus neutralization (e.g. induced by the innate immune system).

The chimeric poxviruses according to the invention were generated via a directed evolution method. The starting pool consisted in a mix of sixteen different poxvirus strains: Rabbitpox virus strain Utrecht (RPX), Cowpox virus strain Brighton (CPX), Vaccinia virus strain Copenhagen (COP), Vaccinia virus strain Western Reserve (WR), Vaccinia virus strain Wyeth (WY), Modified Vaccinia virus strain Ankara (MVA), Raccoonpox virus strain Herman (RCN), ORF virus strain NZ2 (ORF), Pseudocowpox virus strain TJS (PCP), Bovine Papular stomatitis virus strain Illinois 721 (BPS), Myxoma virus strain Lausanne (MYX), Squirrelpox virus strain Kilham (SQF), Fowlpox virus strain FP9 (FPV), Swinepox virus strain Kasza (SPV), Yaba-like disease virus strain Davis (YLD) and Cotia virus strain SP An 232 (CTV).

Most of these parental poxvirus strains are known to be oncolytic to varying degrees of intensity, but it is not the case of MVA, FPV and SPV, which are known not to replicate efficiently in mammalian cells and are therefore not oncolytic (Ricordel et al., 2018, Oncotarget vol 9, 35891-35906; Guse et al., 2011, Expert Opinion Biol. Ther.11 (5): 595-608). MVA and FPV are instead known for their high safety profile in humans, which is why they have been included in the starting mix of poxvirus strains.

After exposure of this mix to a directed evolution method, the inventors selected a chimeric poxvirus, named POXSTG19503 (thereafter referred as “wild-type chimeric poxvirus according to the invention”), with enhanced oncolytic properties and therapeutic index in vitro and better spreading capacity in vivo due to improved syncytia formation and EEV-secretion capacities, compared to its parental strains, and unexpectedly higher EEV-secretion capacity compared to the Vaccinia virus strain IHD-J and rabbitpox virus known as high EEV producer strains of poxvirus. Even more, the inventors selected the said chimeric poxvirus, named POXSTG19503, which induces a superior specific T cell response against tumor.

More precisely, POXSTG19503 is a chimeric orthopoxvirus because it comprises nucleic acid fragments originating only from orthopoxviruses: Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA). Unexpectedly, the enhanced oncolytic activity was present even though a significant proportion of the genome of the chimeric poxvirus was derived from MVA. Importantly, contrary to deVV5, POXSTG19503 showed enhanced anticancer efficiency in vivo.

The inventors also showed that it was possible to delete one or more genes of the chimeric poxviruses without altering its ability to destroy cancer cells, to secrete a high percentage of EEV particles or to induce syncytia formation. This/these deletion(s) (genes encoding TK and/or RR) further improved the viruses' safety, and modified viruses still had stronger oncolytic properties than their parental correspondingly deleted strains.

The inventors also explored the feasibility of the recombination approach using said chimeric poxviruses as viral vectors, and thus constructed armed oncolytic chimeric poxviruses encoding FCU1 or interleukin (IL-12). It appeared that the insertion of said transgenes in the chimeric poxviruses did not alter their anticancer activities. Moreover, it appeared that, due to their metabolism, and more particularly to their rapid replication and their ability to infect a high number of cells due to the chimeras' increased capacity to produce EEV and to induce syncytia formation, said recombinant chimeric poxviruses expressed higher rates of transgenes than their parental vaccinia virus strain Copenhagen expressing the same transgenes.

Based on these results, one may anticipate that the chimeric poxviruses of the invention may be successfully used as a therapeutical solution, especially for treatment of proliferative diseases, and for replacing the existing oncolytic viruses. They have a better efficiency profile in vivo, while remaining safe for use. The chimeric poxviruses of the invention may also be advantageous for treating cancers refractory or resistant to poxvirus-based therapy. The chimeras of the invention can also be exploited in combination with additional anticancer therapy/ies.

Other and further aspects, features and advantages of the present invention will be apparent from the following description of the presently preferred embodiments of the invention. These embodiments are given for the purpose of disclosure.

### SUMMARY OF THE INVENTION

In a first aspect, the invention provides a chimeric poxvirus (optionally variant and/or recombinant), wherein said chimeric poxvirus comprises a nucleic acid sequence having a sequence identity of at least 96,6%, preferably at least 96,7%, at least 96,8%, at least 96,9%, at least 97%, at least 97,1%, at least 97,2%, at least 97,3%, at least 97,4%, at least 97,5%, at least 97,6%, at least 97,7%, at least 97,8%, at least 97,9%, at least 98%, at least 98,1%, at least 98,2%, at least 98,3%, at least 98,4%, at least 98,5%, at least 98,6%, at least 98,7%, at least 98,8%, at least 98,9%, at least 99%, at least 99,1%, at least 99,2%, at least 99,3%, at least 99,4%, at least 99,5%, at least 99,6%, at least 99,7%, at least 99,8%, at least 99,9%, at least 99,91%, at least 99,92%, at least 99,93%, at least 99,94%, at least 99,95%, at least 99,96%, at least 99,97%, at least 99,98%, at least 99,99%, or even 100% of identity with SEQ ID NO:1.

In one embodiment, the chimeric poxvirus of the invention is the chimeric poxvirus POXSTG19503 clone 7 deposited at the Collection Nationale de Cultures de Microorganismes (CNCM) on 20 Oct. 2022 under Accession Number CNCM I-5913.

In the present disclosure, the chimeric poxvirus deposited under Accession Number CNCM I-5913 is also referred as POXSTG19503.

In a preferred embodiment, the chimeric poxvirus (optionally variant and/or recombinant) of the invention comprises nucleic acid fragments from Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA). In a specific embodiment, said chimeric poxvirus (optionally variant and/or recombinant) may comprise a glutamic acid in position 151 of the protein encoded by the A34R gene, or a valine in position 19 of the protein encoded by the A34R gene, or both. In a further specific embodiment, said chimeric virus (optionally variant and/or recombinant) may be partially or totally defective in the A56R locus, in particular may comprise the A56R gene from Rabbitpox virus, preferably from the parental Rabbitpox virus strain Utrecht (RPX). The invention also provides derivatives of any chimeric poxvirus, including recombinant derivatives (i.e.: further comprising one or more heterologous nucleic acid(s) of interest), variant derivatives defective in one or more loci, notably defective in the J2R locus (in particular derivatives in which the J2R locus has been deleted) or deficient in the J2R locus and in the 14L and/or F4L locus (in particular derivatives in which the J2R locus and the 14L and/or F4L locus has been deleted), and recombinant variant derivatives defective in one or more loci as indicated above and further comprising one or more heterologous nucleic acid(s) of interest. A recombinant (optionally variant) chimeric poxvirus according to the invention may encode one or more polypeptide(s) of therapeutic interest, which are preferably selected from polypeptides capable of reinforcing the oncolytic nature of the chimeric poxvirus, polypeptides capable of potentiating anti-tumor efficacy, antigens for inducing or activating an immune humoral and/or cellular response, and permease. In a preferred embodiment, the recombinant chimeric poxvirus according to the invention may encode an interleukin. More preferably, said interleukin is an IL-12.

In a second aspect, the invention provides a chimeric poxvirus (optionally variant and/or recombinant), wherein, for at least one tumor, the oncolytic power of said chimeric poxvirus (optionally variant and/or recombinant) is higher than the oncolytic power measured in the same conditions and at the same time post-infection of at least one of the oncolytic parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY) and

Vaccinia virus strain Western Reserve (WR), wherein for a given tumor, a given virus, given conditions and a given time post-infection, an oncolytic power OP(tumor, virus, conditions, time post-infection) is defined as:

$$OP ( tumor , virus , conditions , time post - infection ) = ( 100 - percentage of surviving tumor cells after infection by virus ) .$$

In a preferred embodiment, for at least one tumor, the oncolytic power of said chimeric poxvirus (optionally variant and/or recombinant) is higher than the oncolytic power measured in the same conditions and at the same time post-infection of the parental Vaccinia virus strain Copenhagen (COP) (optionally variant and/or recombinant) or the parental Rabbitpox virus strain Utrecht (RPX) (optionally variant and/or recombinant), two particularly oncolytic poxvirus strains. More preferably, for at least one tumor, the oncolytic power of said chimeric poxvirus (optionally variant and/or recombinant) is higher than the oncolytic power measured in the same conditions and at the same time post-infection of at least two of the oncolytic parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR). For instance, for at least one tumor, the oncolytic power of said chimeric poxvirus (optionally variant and/or recombinant) is higher than the oncolytic powers measured in the same conditions and at the same time post-infection of the parental Vaccinia virus strain Copenhagen (COP) (optionally variant and/or recombinant) and the parental Rabbitpox virus strain Utrecht (RPX) (optionally variant and/or recombinant). In a more preferred embodiment, for at least one tumor, the oncolytic power of said chimeric poxvirus (optionally variant and/or recombinant) is higher than the oncolytic power measured in the same conditions and at the same time post-infection of at least three, more preferably higher than at least four, and even more preferably higher than each of the five oncolytic parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR).

In a third aspect, the invention provides a chimeric poxvirus (optionally variant and/or recombinant), wherein for at least one healthy cell (preferably primary cell), the viral replication in the healthy cell of said chimeric poxvirus (optionally variant and/or recombinant) is lower than that of at least one of the five oncolytic parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR).

Preferably, for at least one healthy cell (preferably primary cell), the viral replication in the healthy cell of said chimeric poxvirus (optionally variant and/or recombinant) is lower than that of the parental Vaccinia virus strain Copenhagen (COP) (optionally variant and/or recombinant). More preferably, for at least one healthy cell (preferably primary cell), the viral replication in the healthy cell (preferably primary cell) of said chimeric poxvirus (optionally variant and/or recombinant) is lower than that of at least two, more preferably at least three, more preferably at least four, and even more preferably each of the five oncolytic parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR).

In a fourth aspect, the invention provides a chimeric poxvirus (optionally variant and/or recombinant), wherein, for at least one organ, the therapeutic index of said chimeric poxvirus (optionally variant and/or recombinant) is higher than the therapeutic index measured in the same conditions and at the same time post-infection of at least one of the five oncolytic parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) wherein for a given organ, a given tumor, a given virus, given conditions and a given time post-infection, the therapeutic index TI(organ, tumor, virus, conditions, time post-infection) is defined as:

TI(organ, tumor, virus, conditions, time post-infection)=(replication of virus in organ tumor cells/replication of virus in organ healthy cells).

In a preferred embodiment, for at least one organ, the therapeutic index of said chimeric poxvirus (optionally variant and/or recombinant) is higher than the therapeutic index measured in the same conditions and at the same time post-infection of the parental Vaccinia virus strain Copenhagen (optionally variant and/or recombinant). More preferably, for at least one organ, the therapeutic index of said chimeric poxvirus (optionally variant and/or recombinant) is higher than the therapeutic index measured in the same conditions and at the same time post-infection of at least two of, more preferably at least three of, more preferably at least four of, and even more preferably each of the five oncolytic parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR).

In a fifth aspect, the invention provides a chimeric poxvirus (optionally variant and/or recombinant), wherein for at least one producer cell (preferably a tumor cell), the extracellular-enveloped virus (EEV)-secretion capacity (SC) (abbreviated as EEV-SC) of said chimeric poxvirus (optionally variant and/or recombinant) is higher than the EEV-SC measured in the same conditions and at the same time post-infection of at least one of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA), wherein for a given virus, a given producer cell, given conditions and a given time after viral infection, said EEV-SC is the ratio of extracellular enveloped virus (EEV) to the total forms of the virus (extracellular enveloped virus (EEV) and intracellular mature virus (IMV) form of the virus), defined as:

EEV-SC (virus, producer cell, conditions, time post-infection)=number of EEV particles/number of (EEV+IMV) particles.

In a preferred embodiment, the invention provides a chimeric poxvirus (optionally variant and/or recombinant), wherein for at least one producer cell (preferably a tumor cell), the EEV-SC of said chimeric poxvirus (optionally variant and/or recombinant) is higher than the EEV-SC measured in the same conditions and at the same time post-infection of the parental Vaccinia virus strain Copenhagen (COP) (optionally variant and/or recombinant) or the parental Rabbitpox virus strain Utrecht (RPX) (optionally variant and/or recombinant). More preferably, for at least one producer cell (preferably a tumor cell), the EEV-SC of said chimeric poxvirus (optionally variant and/or recombinant) is higher than the EEV-SC measured in the same conditions and at the same time post-infection of at least two of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA). For instance, for at least one producer cell (preferably a tumor cell), the EEV-SC of said chimeric poxvirus (optionally variant and/or recombinant) is higher than the EEV-SCs measured in the same conditions and at the same time post-infection of the parental Vaccinia virus strain Copenhagen (COP) (optionally variant and/or recombinant) and the parental Rabbitpox virus strain Utrecht (RPX) (optionally variant and/or recombinant). In a more preferred embodiment, for at least one producer cell (preferably a tumor cell), the EEV-SC of said chimeric poxvirus (optionally variant and/or recombinant) is higher than the EEV-SC measured in the same conditions and at the same time post-infection of at least three, more preferably at least four, at least five, and more preferably each of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA). Alternatively, or in combination, for at least one producer cell (preferably a tumor cell), the EEV-SC of said chimeric poxvirus (optionally variant and/or recombinant) is higher than the EEV-SC measured in the same conditions and at the same time post-infection of Vaccinia virus strain IHD-J.

Preferably, in said fifth aspect, for at least one tumor, the spreading capacity of said chimeric poxvirus (optionally variant and/or recombinant) is higher than the spreading capacity measured in the same conditions and at the same time post-infection of at least one of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA), wherein for a given virus, producer cell, conditions and time post-infection, the spreading capacity is defined as the capacity of the virus to disseminate between cells (e.g. tumor cells) or between tumors (e.g. spreading from an injected tumor to a distal tumor). It is known that the spreading capacity is related to the formation of EEV particles: the more EEV a virus can generate, the higher its capacity to spread. The spreading capacity can be evaluated by various techniques well known to those skilled in the art, including Virus Comet Assay, said assay allowing the evaluation of comet tail formation (e.g.: number of comets, size of the comet tail). In some cases, an increase in the number of comets or size of the comet tail can indicate an increase in the amount of EEV relative to IMV forms of a viral strain.

In a preferred embodiment, for at least one tumor, the spreading capacity of said chimeric poxvirus (optionally variant and/or recombinant) is higher than the spreading capacity measured in the same conditions and at the same time post-infection of the parental Vaccinia virus strain Copenhagen (COP) (optionally variant and/or recombinant) or the parental Rabbitpox virus strain Utrecht (RPX) (optionally variant and/or recombinant). More preferably, for at least one tumor, the spreading capacity of said chimeric poxvirus (optionally variant and/or recombinant) is higher than the spreading capacity measured in the same conditions and at the same time post-infection of at least two of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA). For instance, for at least one tumor, the spreading capacity of said chimeric poxvirus (optionally variant and/or recombinant) is higher than the spreading capacities measured in the same conditions and at the same time post-infection of the parental Vaccinia virus strain Copenhagen (COP) (optionally variant and/or recombinant) and the parental Rabbitpox virus strain Utrecht (RPX) (optionally variant and/or recombinant). In a more preferred embodiment, for at least one tumor, the spreading capacity of said chimeric poxvirus (optionally variant and/or recombinant) is higher than the spreading capacity of at least three, preferably at least four, more preferably at least five or even more preferably each of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY) Vaccinia virus strain Western Reserve (WR) and Modified Vaccinia virus strain Ankara (MVA).

In a sixth aspect, the invention provides a chimeric poxvirus (optionally variant and/or recombinant), wherein, for at least one poxvirus-specific antibody and tumor, the neutralization rate of said chimeric poxvirus (optionally variant and/or recombinant) is lower than the neutralization rate measured in the same conditions and at the same time post-infection of at least one of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA), wherein for a given virus, a given tumor, a given poxvirus-specific antibody, given conditions and a given time post-infection, the neutralization rate (NT(virus, tumor, conditions, time post-infection)) measures the antibody-induced inhibition of the virus' oncolytic power and is defined as:

NT(virus, tumor, Poxvirus-specific antibody, conditions, time post-infection)=EC50 (with poxvirus-specific antibody)/EC50 (without poxvirus-specific antibody).

In a preferred embodiment, for at least one poxvirus-specific antibody and tumor, the neutralization rate of said chimeric poxvirus (optionally variant and/or recombinant) is lower than the neutralization rate measured in the same conditions and at the same time post-infection of the parental Vaccinia virus strain Copenhagen (COP) (optionally variant and/or recombinant). More preferably, for at least one poxvirus-specific antibody and tumor, the neutralization rate of said chimeric poxvirus (optionally variant and/or recombinant) is lower than the neutralization rate of at least two, preferably at least three, more preferably at least four, more preferably at least five, and even more preferably each of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA).

In a seventh aspect, the invention provides a chimeric poxvirus (optionally variant and/or recombinant), wherein the complement-mediated virus neutralization rate of said chimeric poxvirus (optionally variant and/or recombinant) is lower than the complement-mediated virus neutralization rate measured in the same conditions of at least one of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA), wherein for a given virus and given conditions the complement-mediated virus neutralization rate (CMV-NT(virus, conditions)) measures the complement induced inhibition of the virus' oncolytic power and is defined as:

CMV-NT(virus, conditions)=virus titer (human serum)/virus titer (heat-inactivated serum).

In a preferred embodiment, the complement-mediated virus neutralization rate of said chimeric poxvirus (optionally variant and/or recombinant) is lower than the complement-mediated virus neutralization rate measured in the same conditions of the parental Vaccinia virus strain Copenhagen (COP) (optionally variant and/or recombinant). More preferably, the complement-mediated virus neutralization rate of said chimeric poxvirus (optionally variant and/or recombinant) is lower than the complement-mediated virus neutralization rate measured in the same conditions of at least two, preferably at least three, more preferably at least four, more preferably at least five, and even more preferably each of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA).

In an eighth aspect, the invention provides a chimeric poxvirus (optionally variant and/or recombinant), wherein for at least one producer cell (preferably a tumor cell), the syncytia formation capacity of said chimeric poxvirus (optionally variant and/or recombinant) is higher than the syncytia formation capacity measured in the same conditions and at the same time post-infection of at least one of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA).

In a preferred embodiment, the invention provides a chimeric poxvirus (optionally variant and/or recombinant), wherein for at least one producer cell (preferably a tumor cell), the syncytia formation capacity of said chimeric poxvirus (optionally variant and/or recombinant) is higher than the syncytia formation capacity measured in the same conditions and at the same time post-infection of the parental Vaccinia virus strain Copenhagen (COP) (optionally variant and/or recombinant). More preferably, for at least one producer cell (preferably a tumor cell), the syncytia formation capacity of said chimeric poxvirus (optionally variant and/or recombinant) is higher than the syncytia formation capacity measured in the same conditions and at the same time post-infection of at least two of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA). For instance, in a preferred embodiment, for at least one producer cell (preferably a tumor cell), the syncytia formation capacity of said chimeric poxvirus (optionally variant and/or recombinant) is higher than the syncytia formation capacity measured in the same conditions and at the same time post-infection of at least two, preferably at least three, more preferably at least four, at least five, and more preferably each of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA).

The invention also provides a chimeric poxvirus (optionally variant and/or recombinant) combining features of the chimeric poxviruses (optionally variant and/or recombinant) described above in aspects one to eight.

The invention also relates to a chimeric poxvirus obtained or obtainable by a specific method of directed evolution as described below.

In a nineth aspect, the invention provides a method of directed evolution for obtaining a chimeric poxvirus with high anticancer activity, said method comprising:
(i) infecting a first tumor cell line with a plurality of parental poxvirus strains, wherein said tumor cell line is permissive for each parental poxvirus strain, so as to obtain a first infected tumor cell line;
(ii) amplifying said parental poxvirus strains on said first infected tumor cell line of step (i) during at least 12h (preferably at least 24h) and at most 3 days, so as to obtain one or more distinct chimeric poxviruses through homologous genomic recombination between at least two of said parental poxvirus strains in supernatant;
(iii) collecting the supernatant at the end of step (ii) containing the one or more distinct chimeric poxvirus(es);
(iv) infecting a second tumor cell line with the one or more distinct chimeric poxvirus(es) of the supernatant of step (iii), wherein said second tumor cell line is permissive for each parental poxvirus strain of steps (i) and (ii), so as to obtain a second infected tumor cell line;
(vₐ) amplifying the one or more distinct chimeric poxvirus(es) of step (iv) on said second infected tumor cell line of step (iv) during preferably at least 12 h and at most 24 h and then collecting the supernatant;
(vi) selecting one or more distinct chimeric poxvirus(es) of step (vₐ) having, for at least one third tumor cell line, an oncolytic power higher than the oncolytic power measured in the same conditions and at the same time post-infection of at least one, preferably several, and more preferably all the parental oncolytic poxvirus strains in the first tumor cell line of step (i) and/or in the second tumor cell line of step (iv), wherein for a given tumor, a given virus, given conditions and a given time post-infection, an oncolytic power OP(tumor, virus, conditions, time post-infection) is defined as:
OP(tumor, virus, conditions, time post-infection)=(100-percentage of surviving tumor cells after infection by virus).

More particularly, said method of directed evolution comprises the following steps:
(i) infecting a first tumor cell line with a plurality of parental poxvirus strains, wherein said tumor cell line is permissive for each parental poxvirus strain, so as to obtain a first infected tumor cell line;
(ii) amplifying said parental poxvirus strains on said first infected tumor cell line of step (i) during at least 12h (preferably at least 24h) and at most 3 days, so as to obtain one or more distinct chimeric poxviruses through homologous genomic recombination between at least two of said parental poxvirus strains in supernatant;
(iii′) collecting the supernatant at the end of step (ii) containing the one or more distinct chimeric poxvirus(es), and performing a 5 to 20-fold dilution series so as to obtain at least two diluted supernatants each containing one or more chimeric poxvirus(es);
(iv′) infecting at least two samples of a second tumor cell line with the one or more distinct chimeric poxvirus(es) of each of the diluted supernatants of step (iii′), wherein said second tumor cell line is permissive for each parental poxvirus strain of steps (i) and (ii), so as to obtain at least two samples of the second infected tumor cell line;
(v′ₐ) amplifying the one or more distinct chimeric poxvirus(es) of each of the at least two samples of the second infected tumor cell line of step (iv′) on the second infected tumor cell line of step (iv′) during preferably at least 12 h and at most 24h;
(v′_{b}) collecting the supernatant from the sample of infected second tumor cell line infected with the less diluted supernatant that shows no sign of cytopathic effect, and performing a 5 to 20-fold dilution series;
(v′_{c}) repeating steps (iv), (v′ₐ) and (v′_{b}) until one or more distinct chimeric poxvirus(es) meeting the selection criteria of step (vi) is obtained; and
(vi) selecting one or more distinct chimeric poxvirus(es) of step (v′_{c}) having, for at least one third tumor cell line, an oncolytic power higher than the oncolytic power measured in the same conditions and at the same time post-infection of at least one, preferably several, and more preferably higher than all the parental oncolytic poxvirus strains in the first tumor cell line of step (i) or in the second tumor cell line of step (iv′), wherein for a given tumor, a given virus, given conditions and a given time post-infection an oncolytic power OP(tumor, virus, conditions, time post-infection) is defined as:

$$OP ( tumor , virus , conditions , time post - infection ) = ( 100 - percentage of surviving tumor cells after infection by virus ) .$$

Even more particularly, said method of directed evolution comprises the following steps:
i) infecting a first tumor cell line with a plurality of parental poxvirus strains, wherein said tumor cell line is permissive for each parental poxvirus strain, so as to obtain a first infected tumor cell line;
(ii) amplifying said parental poxvirus strains on said first infected tumor cell line of step (i) during at least 12h (preferably at least 24h) and at most 3 days, so as to obtain one or more distinct chimeric poxviruses through homologous genomic recombination between at least two of said parental poxvirus strains;
(iii″ₐ) collecting both cells and supernatant at the end of step (ii) containing one or more distinct chimeric poxvirus(es);
(iii″_{b}) infecting a second tumor cell line with both cells and supernatant containing one or more distinct chimeric poxvirus(es) of step (iii″ₐ), wherein said second tumor cell line is permissive for each parental poxvirus strain of steps (i) and (ii), so as to obtain a second infected tumor cell line;
(iii″_{c}) amplifying one or more distinct chimeric poxvirus(es) of step (iii″b) on said second infected tumor cell line of step (iii″_{b}) during at least 48h (preferably at least 72h) and at most 3 days;
(iii″_{d}) collecting a fraction of the cells and supernatant containing one or more distinct chimeric poxvirus(es) of step (iii″_{c});
(iii″ₑ) repeating steps (iii″_{b}), (iii″_{c}) and (iii″ₐ) at least one time;
(iii″_{f}) collecting the supernatant at the end of step (iii″ₑ) containing one or more distinct chimeric poxvirus(es), and performing a 5 to 20-fold dilution series so as to obtain at least two diluted supernatants;
(iv″) infecting at least two samples of a third tumor cell line with the one or more distinct chimeric poxvirus(es) of each of the diluted supernatants of step (iii″_{f}), wherein said third tumor cell line is permissive for each parental poxvirus strain of steps (i) and (ii) so as to obtain at least two samples of the third infected tumor cell line;
(v″ₐ) amplifying the one or more distinct chimeric poxvirus(es) of each of the at least two samples of the third infected tumor cell line of step (iv″) on a third infected tumor cell line of step (iv″) during at least 12 h and at most 24h;
(v″_{b}) collecting the supernatant from the sample of third infected tumor cell line infected with the less diluted supernatant that shows no sign of cytopathic effect, and performing a 5 to 20-fold dilution series;
(v″_{c}) repeating steps (iv″), (v″ₐ) and (v″_{b}) until one or more distinct chimeric poxvirus(es) meeting the selection criteria of step (vi) is obtained; and
(vi) selecting one or more distinct chimeric poxvirus(es) of step (v″_{c}) having, for at least one fourth tumor cell line, an oncolytic power higher than the oncolytic power measured in the same conditions and at the same time post-infection of at least one, preferably several, and more preferably all the parental oncolytic poxvirus strains in the tumor cell line of steps (iv″), wherein for a given tumor, a given virus, given conditions and a given time post-infection, an oncolytic power OP(tumor, virus, conditions, time post-infection) is defined as:

OP(tumor, virus, conditions, time post-infection)=(100-percentage of surviving tumor cells after infection by virus).

In a specific embodiment, said parental poxviruses strains used in first step (i) are selected in the group consisting of Rabbitpox virus strain Utrecht (RPX), Cowpox virus strain Brighton (CPX), Vaccinia virus strain Copenhagen (COP), Vaccinia virus strain Western Reserve (WR), Vaccinia virus strain Wyeth (WY), Modified Vaccinia virus strain Ankara (MVA), Raccoonpox virus strain Herman (RCN), ORF virus strain NZ2 (ORF), Pseudocowpox strain TJS (PCP), Bovine Papular stomatitis virus strain Illinois 721 (BPS), Myxoma virus strain Lausanne (MYX), Squirrelpox virus strain Kilham (SQF), Fowlpox virus strain FP9 (FPV), Swinepox virus strain Kasza (SPV), Yaba-like disease virus strain Davis (YLD) and Cotia virus strain SP An 232 (CTV). More preferably, said parental poxviruses strains used in first step (i) comprise at least one, preferably at least two, at least three, at least four, at least five, or even all six of Rabbitpox virus strain Utrecht (RPX), Cowpox virus strain Brighton (CPX), Vaccinia virus strain Copenhagen (COP), Vaccinia virus strain Western Reserve (WR), Vaccinia virus strain Wyeth (WY) and Modified Vaccinia virus strain Ankara (MVA). The inventors showed that these 6 parental poxviruses were able to recombine with each other and the parental poxviruses strains used in first step (i) may thus be selected in this more limited group. In an embodiment, the parental poxviruses strains used in first step (i) may notably consist of Rabbitpox virus strain Utrecht (RPX), Cowpox virus strain Brighton (CPX), Vaccinia virus strain Copenhagen (COP), Vaccinia virus strain Western Reserve (WR), Vaccinia virus strain Wyeth (WY) and Modified Vaccinia virus strain Ankara (MVA).

In any one of the above methods of directed evolution, the first, second and optionally third permissive tumor cell lines may be different or the same. They are from higher mammal's origins, preferably said permissive tumor cell lines used as first, second, third and optionally fourth tumor cell line in the above methods are selected in the group consisting of A549, CAL-33, HepG2, HCT116, Hela, SK-MEL-1, PANC-1, Hs746T, SK-OV-3 and CV-1, with a preference for A549. In a preferred embodiment, the first, second, third and optionally fourth permissive tumor cell lines are the same and are preferably all A549 lung cancer cell line.

In a further aspect, the present invention also relates to a variant chimeric poxvirus, i.e.: a chimeric poxvirus according to the invention, which has been engineered by altering one or more viral gene(s) (e.g.: deletion of the TK encoding gene).

In a further aspect, the present invention also relates to a recombinant chimeric poxvirus, i.e.: a chimeric poxvirus according to the invention, which comprises one or more heterologous transgene(s).

In a further aspect, the present invention also relates to a recombinant variant chimeric poxvirus, i.e.: a chimeric poxvirus according to the invention, which has been engineered by altering one or more viral gene(s) (e.g.: deletion of the TK encoding gene) and comprises one or more heterologous transgene(s).

In a further aspect, the present invention also concerns a process for producing a chimeric (optionally variant and/or recombinant) poxvirus, comprising at least the steps of:
(i) infecting a producer cell with the chimeric (optionally variant and/or recombinant) poxvirus according to the invention as disclosed herein;
(ii) culturing said infected producer cell under conditions which are appropriate for enabling chimeric poxvirus (optionally variant and/or recombinant) to be produced, and;
(iii) recovering said chimeric poxvirus (optionally variant and/or recombinant) from the producer cell culture.

Optionally, the recovered chimeric (optionally variant and/or recombinant) poxvirus can be purified at least partially.

Another aspect of the present invention relates to an isolated nucleic acid encoding the chimeric (optionally variant and/or recombinant) poxvirus of the invention.

In another aspect of the invention is provided a composition comprising the chimeric (optionally variant and/or recombinant) poxvirus of the invention and a pharmaceutically acceptable vehicle. In one embodiment, the chimeric (optionally variant and/or recombinant) poxvirus is preferably formulated for parenteral route administration, with a preference for intravenous or intratumoral route.

Another aspect of the present invention relates to the chimeric (optionally variant and/or recombinant) poxvirus or the composition of the invention, for use as a drug, preferably for the treatment of a proliferative disease. In a preferred embodiment, said proliferative disease is selected from cancers, tumors and restenosis.

Another aspect of the present invention relates to a method for treating a disease in a subject in need thereof comprising the administration to said subject of the chimeric poxvirus (optionally variant and/or recombinant) or the composition according to the invention. In one embodiment, said disease is a proliferative disease, wherein said proliferative disease is preferably selected from cancers, tumors and restenosis.

### DESCRIPTION OF THE FIGURES

In order to make the reading process easier, Table 1 is provided, comprising the description of the code references used in the Figures and Examples. Table 1 is provided in the Example section.

FIG. 1: Oncolytic effect of POXSTG19503 on a panel of tumor cells. Cells (3×10⁵ cells/well plated in 6-well culture dishes) were infected with the indicated MOI and cell viability was determined 5 days later by trypan blue exclusion. The parental COP was used as reference. The results are presented as a mean of triplicate experiments±SD.

FIG. 2: Genome analysis. (A) Annotated POXSTG19503 mono-ITR genome. Large grey arrows highlight the segments longer than 500 nucleotides 100% identical to parental virus genomes, indicated by labels and greyscale code from the upper right legend. The 3′ ITR is indicated by the terminal black arrow. (B) Region 40 kb-60 kb extracted from the global alignment of the core-region of POXSTG19503 and the parental genomes. Large grey arrows highlight the segments longer than 500 nucleotides 100% identical to parental virus genomes, indicated by labels and greyscale code from the upper right legend. Automatically detected open reading frames are reported by smaller dark grey arrows in the lower part of each lane.

FIG. 3: Oncolytic effect of POXSTG19508 on A549 (A), HCT116 (B) and HepG2 (C) tumor cells. Cells (3×10⁵ cells/well plated in 6-well culture dishes) were infected with the indicated MOI and cell viability was determined 4 days later by trypan blue exclusion. The 6 parental poxvirus strains were used as reference. The results are presented as a mean of triplicate experiments±SD.

FIG. 4: Replication in tumor cells and in primary human cells. (A) HepG2 tumor cells were infected at MOI 10⁻⁵ and harvested 3 days post infection. Human primary hepatocytes were infected at MOI 10⁻⁴ and harvested 3 days post infection. 3D Phenion FT skin models were infected with 1.10⁵ PFU (plaque-forming unit) and harvested 7 days post infection. Viral progeny production was determined by plaque titration. Results are expressed as viral fold amplification (corresponding to output/input ratio). The results are presented as a mean of triplicate experiments±SD. (B) Ratio between viral fold amplification obtained in HepG2 hepatocarcinoma cells and hepatocytes 3 days post infection. Values are represented as the mean of three individual determinations.

FIG. 5: Functionality of FCU1 expressed by the chimeric poxvirus POXSTG19508. Conversion of 5-fluorocytosine (5-FC) to 5-fluorouracil (5-FU) and release of 5-FU in the cell culture supernatant. A549 tumor cells were infected with the indicated vector at a MOI 10⁻⁴ and then incubated with 1 mM 5-FC added 6 hours post infection. The relative concentration of 5-FC and 5-FU in the culture supernatant was measured by HPLC from day 1 to day 3 post infection. The results are expressed as the percentage of 5-FU released relative to the total amount of 5-FC+5-FU. Values are represented as the mean of three individual determinations±SD.

FIG. 6: Production of EEV form and total progeny viruses at early times after infection of a A549 monolayer. A549 cells were infected with VVTG17111 or POXSTG19508 at MOI of 0.1. Supernatant or cell fraction were collected at 16- and 24-hours post-infection. Virus titers from the supernatants alone (EEV) and from both supernatants and cells (IMV+EEV, total progeny virus) at 16 hours (A) and 24 hours (B) post-infection are presented. (C) Ratio of EEV to IMV at 16- and 24-hours post-infection. The results are presented as a mean of triplicate experiments±SD.

FIG. 7: Representative images of comets. The indicated viruses were plated on monolayers of A549 cells. After 2 days, plaques were imaged using fluorescence microscopy (GFP). After being imaged, cells were stained with crystal violet (CV).

FIG. 8: Production of EEV form and total progeny viruses at early times after infection of a A549 monolayer. A549 cells were infected with the indicated viruses at MOI of 0.1. Supernatant or cell fraction were collected at 16- and 24-hours post-infection. Virus titers from the supernatants alone (EEV) and from both supernatants and cells (IMV+EEV, total progeny virus) at 16 hours (A) and 24 hours (B) post-infection are presented. (C) Ratio of EEV to IMV at 16- and 24-hours post-infection. Values are represented as the mean of three individual determinations±SD.

FIG. 9: VACV neutralization assay. Viability of HCT116 tumor cells infected by TG6002 (A) or POXSTG19508 (B) in presence of patient-derived serum collected 42 days after IV administration of TG6002 (immunized serum). Condition without serum (control) and condition using serum collected from the same patient before administration of TG6002 (pre-immunized serum) were used as negative controls. The half maximal effective concentration (EC50) of the two viruses was determined by infecting HCT116 cells seeded in 96-well plate (1×10⁴ cells/well) with serial dilutions of the indicated virus and evaluating cell death 3 days post-infection by a CellTiter-Blue Cell Viability Assay. Cell viability was determined by comparison to uninfected cell controls. The results are presented as a mean of triplicate experiments±SD. (C) Table of EC50s determined by fitting a sigmoidal dose-response curve from the results obtained in (A) and (B).

FIG. 10: Viral immunostaining in the HCT116 xenograft tumors. (A) Immunostaining of the tumors performed 2, 7 and 16 days after a single intravenous injection of the indicated virus at 1×10⁵ PFU. Cellular DNA was stained in blue with DAPI (medium grey on the pictures) and virus was stained in green (light grey on the pictures). (B) Area density of virus in tumors of 3 mice per group at days 2, 7 and 16 post-injection. The results are presented as a mean of the 3 tumors+SD.

FIG. 11: Viral immunostaining in the B16F10 syngeneic murine tumors. (A) Immunostaining of the tumors performed 3 and 8 days after a single intratumoral injection of the indicated virus at 1×10⁷ PFU. Cellular DNA was stained in blue with DAPI (medium grey on the pictures) and virus was stained in green (light grey on the pictures). (B) Area density of virus in tumors of 3 mice per group at days 3 and 8 post-injection. The results are presented as a mean of the 3 tumors+SD.

FIG. 12: In vivo anti-tumor efficacy of POXSTG19508 in a colorectal xenograft model. Subcutaneous HCT116 tumors were implanted into the right flank of nude mice. On day 15 post-implantation, mice were treated with one intravenous administration of PBS (control), TG6002 or POXSTG19508 (indicated by a vertical arrow) at 3×10⁴ PFU. (A) Tumor growth dynamics of individual mice (n=10 per group). Vertical arrow indicates the systemic injection. (B) Kaplan-Meier survival analysis. Vertical arrow indicates the systemic injection. Significant differences among the group were determined by log-rank test. Ns: not significant.

FIG. 13: In vivo anti-tumor efficacy of POXSTG19508 in a hepatocarcinoma xenograft model. Subcutaneous HepG2 tumors were implanted into the right flank of nude mice. On day 28 post-implantation, mice were treated with one intravenous administration of PBS (control), TG6002 or POXSTG19508 (indicated by a vertical arrow) at 3×10⁵ PFU. (A) Tumor growth dynamics of individual mice (n=10 per group). Vertical arrow indicates the systemic injection. (B) Kaplan-Meier survival analysis. Vertical arrow indicates the systemic injection. Significant differences among the group were determined by log-rank test.

FIG. 14: In vivo anti-tumor efficacy of POXSTG19508 in the CT26 syngeneic murine tumor model. Subcutaneous CT26 tumors were implanted into the right flank of nude mice. On day 7, 9 and 11 post-implantation, mice were treated with daily intratumoral injections of PBS (control), TG6002 or POXSTG19508 (indicated by a vertical arrow) at 1×10⁷ PFU. (A) Tumor growth dynamics of individual mice (n=10 per group). (B) Kaplan-Meier survival analysis. Vertical arrows indicate the intratumoral injections. Significant differences among the group were determined by log-rank test. Ns: not significant.

FIG. 15: In vivo anti-tumor efficacy of POXSTG19508 in a bilateral flank xenograft mouse model. Subcutaneous HCT116 xenografts were injected intratumorally with 1×10⁶ PFU of TG6002, POXSTG19508 or PBS (control) only in the right-sided tumors (n=10 per group). (A) Tumor growth dynamics of injected tumors. (B) Tumor growth dynamics of uninjected tumors. (C) Kaplan-Meier survival analysis. Vertical arrow indicates the IT injection in the right-sided tumors. Significant differences among the group were determined by log-rank test. Ns: not significant. (D) Virus titer in injected and uninjected tumors at days 13 post-virus injection. Each dot represents one tumor. Horizontal bars represent the mean.

FIG. 16: In vivo anti-tumor efficacy of systemic administration of the chimeric poxvirus expressing mIL-12 in the B16F10 syngeneic murine tumor model.

Subcutaneous B16F10 tumors were implanted into the right flank of nude mice. On day 7 and 9 post-implantation, mice were treated with intravenous administration of PBS (control), VVTG19328 or POXSTG19847 at 1×10⁷ PFU. (A) Tumor growth dynamics of individual mice (n=10 per group). (B) Kaplan-Meier survival analysis. Vertical arrows indicate the systemic injections. Significant differences among the group were determined by log-rank test. Ns: not significant.

FIG. 17: Syncytia formation.

HCT116 cells were infected with VVTG17111 or POXSTG19508 at MOI 10⁻² or MOI 10⁻³. Cell morphology was observed at 40h (at MOI 10⁻²) or 60h (at MOI 10⁻³) post-infection by optical and fluorescence microscopy (GFP).

FIG. 18: Specific T cell response measured by IFNγ ELISpot on splenocytes of mice.

Specific T cell response against both tumor and virus measured by IFNγ ELISpot on splenocytes of mice bearing CT26 tumors treated intratumorally with VVTG17111, POXSTG19508 or vehicle as a negative control. Each bar represents the mean+/−SEM of quadruplicate measures of number of spots for 10⁶ splenocytes isolated from individual spleen. * P<0.001.

FIG. 19: Sensitivities to neutralization by complement.

TG6002 and POXSTG19508 were incubated with human serum for 1 h, then used to inoculate Vero cells and incubated for 3 days to allow plaque formation. The numbers of plaques obtained are expressed as percentages of the number of plaques obtained with heat-inactivated human serum. Heat-inactivated serum was used as a negative control. Data represents means of three experiments ±SD.

### DETAILED DESCRIPTION OF THE INVENTION

### General Definitions

The terms used in this specification generally have their ordinary meanings in the art, unless otherwise indicated. Certain terms are discussed below, or elsewhere in the specification, to provide additional guidance to the practitioner in describing the products and methods of the invention and how to use them. Moreover, alternative language and synonyms may be used for any one of the terms discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term.

As used throughout the entire application, the terms “a” and “an” are used in the sense that they mean “at least one”, “at least a first”, “one or more” or “a plurality” of the referenced components or steps. For example, the term “a chimeric poxvirus” encompasses a single chimeric poxvirus as well as a plurality of chimeric poxvirus, including mixtures of different chimeric poxviruses.

The term “one or more” refers to either one or a number above one (e.g.: 2, 3, 4, etc.).

The term “at least” refers to either the number preceded by the expression “at least”, considered as the minimum, or a number above said minimum.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

The term “distinct” means “not identical”, “distinguished as not being the same”. For example, two distinct proteins have different amino-acid sequences, different shapes, etc.

As used herein, when used to define products and compositions, the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. The expression “consisting essentially of” means excluding other components or steps of any essential significance. Thus, a composition consisting essentially of the recited components would not exclude traces, contaminants and pharmaceutically acceptable carriers. “Consisting of” shall mean excluding more than trace elements of other components or steps. In the present description, each time the term “comprising” (or any of its derivatives such as “comprise” and “comprises”) is used, the invention also relates to the same embodiment in which “comprising” (or any of its derivatives such as “comprise” and “comprises”) is replaced by “consisting essentially of” or “consisting of”.

The terms “protein”, “polypeptide”, and “peptide” are used interchangeably and refer to polymers of amino acid residues which comprise at least nine or more amino acids bonded via peptide bonds. The polymer can be linear, branched or cyclic and may comprise naturally occurring and/or amino acid analogues and it may be interrupted by non-amino acids. As a general indication, if the amino acid polymer is more than 50 amino acid residues, it is preferably referred to as a “polypeptide” or a “protein” whereas if it is 50 amino acids long or less, it is referred to as a “peptide”. Proteins, polypeptides and peptides are defined by amino acid sequences.

Within the context of the present invention, the terms “nucleic acid”, “nucleic acid molecule”, “polynucleotide” and “nucleotide sequence” are used interchangeably and define a polymer of any length of either polydeoxyribonucleotides (DNA) (e.g.: cDNA, genomic DNA, plasmids, vectors, viral genomes, isolated DNA, probes, primers and any mixture thereof) or polyribonucleotides (RNA) (e.g.: mRNA, antisense RNA, siRNA) or mixed polyribopolydeoxyribonucleotides. They encompass single or double-stranded, linear or circular, natural or synthetic, modified or unmodified polynucleotides. Moreover, a polynucleotide may comprise non-naturally occurring nucleotides and may be interrupted by non-nucleotide components.

The term “nucleotide” refers to any of various compounds consisting of a sugar, usually ribose or deoxyribose, a purine or pyrimidine base, and one or more phosphates. The expression “nucleotide” designates both ribonucleotides and deoxyribonucleotides.

In a general manner, the term “identity” or “identical” in the context of a virus sample refers to an amino acid to amino acid, or nucleotide to nucleotide correspondence between a polypeptide of the virus and another polypeptide of reference or between a nucleic acid sequence of the virus and another nucleic acid sequence of reference respectively. The percentage of identity between two sequences is a function of the number of identical positions shared by the sequences after optimal global alignment, taking into account the number of gaps which need to be introduced for optimal alignment of the two entire sequences and the length of each gap. Various computer programs and mathematical algorithms are available in the art to determine the percentage of identity between amino acid sequences after optimal global alignment, such as for example the Mafft, ClustalW or ALIGN in Atlas of Protein Sequence and Structure (Dayhoffed, 1981, Suppl., 3:482-9), and the algorithm of Needleman et Wunsh (J. Mol. Biol. 48,443-453, 1970), such computer program and mathematical algorithm of global alignment are selected according to the common general knowledge of a person skilled in the art to perform the more bioinformatically or biologically relevant and optimal global alignment. Programs for determining identity between nucleotide sequences optimal global alignment are also available in specialized data base (e.g.: Genbank, the Wisconsin Sequence Analysis Package, BESTFIT, FASTA and GAP programs).

As used herein, the term “host cell” should be understood broadly without any limitation concerning particular organization in tissue, organ, or isolated cells. Such cells may be of a unique type of cells or a group of different types of cells such as cultured cell lines, healthy cells (preferably primary cells) and dividing cells. In the context of the invention, the term “host cells” includes prokaryotic cells, lower eukaryotic cells such as yeast, and other eukaryotic cells such as insect cells, plant and mammalian (e.g.: human or non-human) cells as well as cells allowing infection and replication of the chimeric poxvirus of the invention (these cells are designated as “permissive cells”). When used for producing the chimeric poxvirus of the invention, permissive cells are referred to “producer cells” are host cells permissive for infection and replication of the chimeric poxvirus of the invention.

The terms “chimeric virus” or “virus chimera” are interchangeable and used according to their ordinary meaning in virology: they refer to a hybrid virus created by joining nucleic acid fragments from two or more different virus strains, which are referred to as “parental viruses”. Chimeric viruses may be obtained through a process of virus directed evolution, in which a mixture of several parental viruses is contacted with producer cells of interest in order to generate recombination events between the genomes of several parental viruses, thus generating a pool of chimeric viruses.

Within the context of the present invention, the term “chimeric gene” defines a hybrid gene formed through the recombination of parts or entire fragments of a gene from two or more different virus strains.

The term “wild-type virus” designates a parental or chimeric virus, wherein said virus has not been engineered by altering one or more gene(s) of the viral genome and does not comprise any heterologous transgene.

The term “variant virus” designates a parental or chimeric virus, wherein said virus has been engineered by altering one or more viral gene(s) (e.g.: deletion of the TK encoding gene).

The term “recombinant virus” designates a parental or chimeric virus, wherein said virus comprises one or more heterologous transgene(s).

The terms “recombinant variant virus” or “variant recombinant virus” are used interchangeably and refer to a parental or chimeric virus, wherein said virus has been engineered by altering one or more viral gene(s) (e.g.: deletion of the TK encoding gene) and comprises one or more heterologous transgene(s).

The term “oncolytic” as used herein refers to the ability of a virus to replicate in dividing cells (e.g.: a proliferative cell such as a cancer cell) with the aim of slowing the growth and/or lysing said dividing cell, either in vitro or in vivo. An oncolytic virus may be characterized by its “oncolytic power”. For a given tumor, a given virus, given conditions and time post-infection, the oncolytic power OP(tumor, virus, conditions, time post-infection) is defined as:

OP(tumor, virus, conditions, time post-infection)=(100-percentage of surviving tumor cells after infection by virus).

For a given tumor and virus, the value of oncolytic power OP(tumor, virus, conditions, time post-infection) may vary depending on the conditions (e.g. MOI, culture medium, tumor cell density, temperature, etc., in particular MOI) and time post-infection, and when the oncolytic powers OP(tumor, virus, conditions, time post-infection) of two viruses are compared, the comparison is made in the same conditions and at the same time post-infection. However, for a given tumor and two given viruses, any suitable conditions (see examples below) and time post-infection may be used for comparison (provided that they are identical for the two viruses), as the hierarchy of oncolytic power will generally remain the same for any suitable conditions and time post-infection when the difference in oncolytic power between the two viruses is sufficient (e.g.: at least a factor 3) or significant. For a given tumor and virus, the oncolytic power may generally be determined in vitro, 3 to 5 days post-infection of tumor cells with the virus at an MOI of 10⁻⁵ to 10⁻².

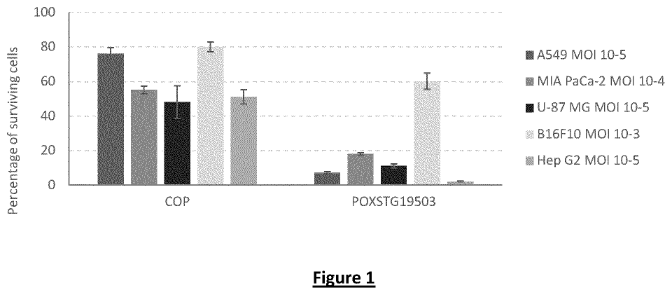

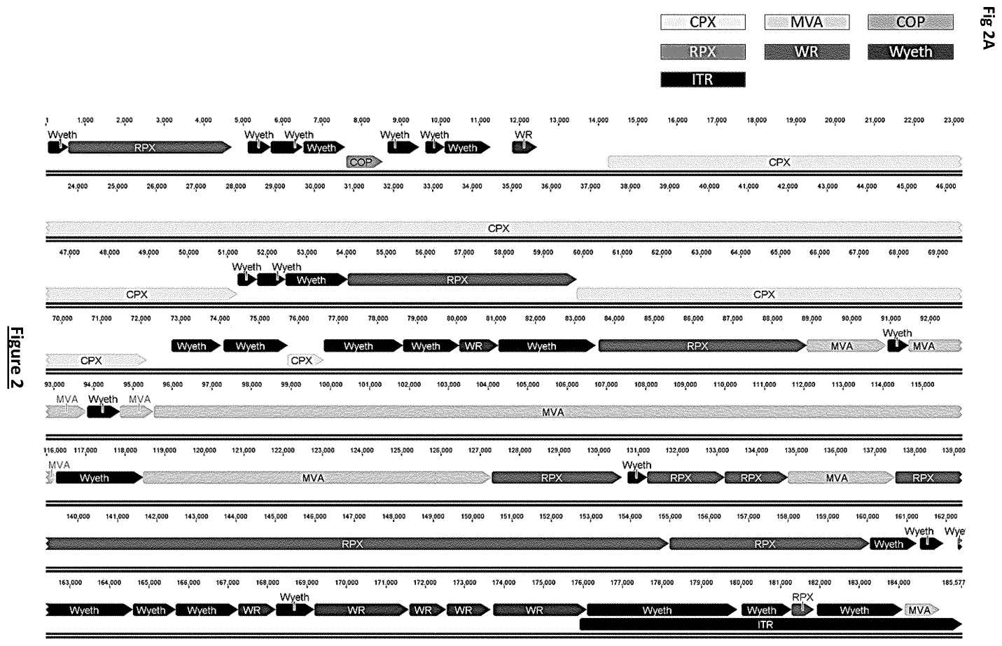

The oncolytic power is expressed as a percentage and represents the percentage of specific tumor cells lysed by a given virus in specific conditions and at a specific time post-infection. For example, 5 days post infection, a vaccinia virus strain Copenhagen at an MOI of 10⁻⁵ has an oncolytic power of 24% on A549 tumor cell cultured at 37° C., 5% CO2, in Dulbecco's Modified Eagle Medium (DMEM) with 10% of Fetal Calf Serum (FCS) s, meaning that 24% of A549 tumor cells were lysed by COP(see FIG. 1). The higher the percentage is, the more oncolytic a given virus for a given tumor is.

The terms “replication”, “viral replication” and “virus replication” refer to the replication of the viral genome in target host cells (e.g.: tumors or healthy cells), or to the synthesis of viral proteins in the target host cells. The steps of a viral life cycle include, but are not limited to, virus attachment to the host cell surface, penetration or entry of the host cell (e.g. through receptor mediated endocytosis or membrane fusion), uncoating (the process whereby the viral capsid is removed and degraded by viral enzymes or host enzymes thus releasing the viral genomic nucleic acid), genome replication, synthesis of viral messenger RNA (mRNA), viral protein synthesis, and assembly of viral ribonucleoprotein complexes for genome replication, assembly of virus particles, post-translational modification of the viral proteins, and release from the host cell by lysis or budding and acquisition of a phospholipid envelope which contains embedded viral glycoproteins. In a cell, there is a viral replication when the viral titer (measured intra- and extra-cellularly) is multiplied by a number higher than 1. In a permissive tumor cell line, the level of virus replication can be low (e.g.: 48 hours post-infection, multiplication of the viral titer by a number higher than 1 and lower than 20000), medium (e.g.: 48 hours post-infection, multiplication of the viral titer by a number comprised between 20000 to 40000) or high (e.g.: 48 hours post-infection, multiplication of the viral titer by a number higher than 40000).

For given tumor or healthy cell (preferably primary cell), the viral replication may generally be determined in vitro, 2 to 8 days post-infection of tumor or healthy cell (preferably primary cell) with the virus at an MOI of 10⁻⁵ to 10⁻².

The term “therapeutic index” represents the ratio between the viral replication on tumor and corresponding healthy cells (i.e.: healthy cells from the same organ). For given organ healthy and tumor cells, a given virus, given conditions and a given time post-infection, the therapeutic index TI(organ healthy, organ tumor, virus, conditions, time post-infection) is defined as follows:

TI(organ healthy, organ tumor, virus, conditions, time post-infection)=(replication of virus in organ tumor cells/replication of virus in organ healthy cells).

For a given organ and virus, the therapeutic index may vary depending on the conditions (e.g.: MOI, culture medium, tumor cell density, temperature, etc., in particular MOI) and time post-infection, and when the therapeutic indexes of two viruses are compared, it is in the same conditions and at the same time post-infection. However, similarly to the oncolytic power, any suitable conditions (see examples below) and time post-infection may be used for comparison (provided that they are identical for the two viruses), as the hierarchy of therapeutic index will generally remain the same for any suitable conditions and time post-infection when the difference in therapeutic index between the two viruses is sufficient. For a given organ and virus, the therapeutic index may generally be determined in vitro 2 to 8 days post-infection of tumor or healthy cells (preferably primary cells) with the virus at an MOI of 10⁻⁵ to 10⁻².

This therapeutic index is improved by an increase of the replicative activity on tumor cells and/or by a decrease of the replication on corresponding healthy cells (preferably primary cells).

The term “extracellular-enveloped virus (EEV)-secretion capacity” abbreviated as “EEV-SC” refers for a given tumor, a given virus, given conditions and a given time post-infection to the percentage of EEV particles among all (EEV+IMV) particles and is defined as:

EEV-SC (tumor, virus, conditions, time post-infection)=number of EEV particles/number of (EEV+IMV) particles For a given tumor and virus, the EEV-SC may vary depending on the conditions (e.g.: MOI, culture medium, tumor cell density, temperature, etc., in particular MOI) and time post-infection, and when the EEV-SC of two viruses are compared, it is in the same conditions and at the same time post-infection. However, similarly to the oncolytic power, any suitable conditions (see examples below) and time post-infection may be used for comparison (provided that they are identical for the two viruses), as the hierarchy of EEV-SC will generally remain the same for any suitable conditions and time post-infection when the difference in EEV-SC between the two viruses is sufficient. For a given organ and virus, the EEV-SC may generally be determined in vitro, 16 to 24 hours post-infection of tumor cells with the virus at an MOI of 10⁻⁴ to 10⁻¹.

The term “syncytia formation capacity” refers to the capacity of a virus to induce the fusion of an infected cell with neighbouring cells, resulting in the formation of multinucleated, enlarged cells referred to as “syncytia”. The dissemination of the virus is both faster and no longer restricted to cells first infected. This results in the infection of more tumor cells than with a virus that does not induce syncytia formation (Burton et al., 2019, Mol Ther Oncolytics, 15, 131-139, Krabbe et al., 2018, Cancers, 10, 216).

The term “spreading capacity” refers to the capacity of the virus to disseminate between cells (e.g.: tumor cells) or between tumors (e.g.: spreading from a first tumor at a first location to a second tumor at a distal second location). It is known that the spreading capacity is related to the formation of EEV and/or syncytia: the more EEV and/or syncytia a virus can generate, the higher its capacity to spread. The spreading capacity can be evaluated by various techniques well known to those skilled in the art, including Virus Comet Assay, said assay allowing the evaluation of comet tail formation (e.g.: number of comets, size of the comet tail). In some cases, an increase in the number of comets or size of the comet tail can indicate an increase in the amount of EEV relative to IMV forms of a viral strain. Said evaluation of comet tail formation for a given tumor and virus may generally be determined in vitro, 16 to 24 hours post-infection of tumor cells with the virus at an MOI of 10⁻⁴ to 10⁻¹.

The terms “viral neutralization rate” and “neutralization rate” are used interchangeably and measure the antiviral antibody-induced inhibition of the virus' oncolytic power. For a given virus, a given tumor, a given poxvirus-specific antibody, given conditions and a given time post-infection, it is defined as:

NT(virus, tumor, poxvirus-specific antibody, conditions, time post-infection)=EC₅₀ (with poxvirus-specific antibody)/EC₅₀ (without poxvirus-specific antibody), wherein said EC₅₀, or half maximal effective concentration is known by the man of the art as the concentration of a drug (here the chimeric poxvirus) which induces a response halfway between the baseline and maximum, or as the concentration required to obtain 50% effect (here 50% tumor cell viability).

For given virus, tumor and poxvirus-specific antibody, the neutralization rate may vary depending on the conditions (e.g.: MOI, culture medium, tumor cell density, temperature, etc., in particular MOI) and time post-infection, and when the neutralization rates of two viruses are compared, it is in the same conditions and at the same time post-infection. However, similarly to the oncolytic power, any suitable conditions (see examples below) and time post-infection may be used for comparison (provided that they are identical for the two viruses), as the hierarchy of neutralization rate will generally remain the same for any suitable conditions and time post-infection when the difference in neutralization rate between the two viruses is sufficient. For a given organ and virus, the neutralization rate may generally be determined in vitro, 3 to 5 days post-infection of tumor cells with the virus at an MOI of 3×10⁻⁵ to 3.

The term “complement-mediated virus neutralization rate” is used to measure the complement induced inhibition of the virus' oncolytic power. For a given virus and given conditions, it is defined as:

CMV-NT(virus, conditions)=virus titer (active serum)/virus titer (heat-inactivated serum), wherein virus titer is assessed by plaque assays.

By “active serum”, it is referred to serum that contains active complement components. Serum normally contains active complement components, but these components may be altered and rendered inactive by to long storage or heat. By “heat-inactivated serum”, it is referred to serum that has been submitted to a heating treatment (generally to 56° C. for 30 minutes) that has inactivated the complement components present in the serum.

Plaques assays are well known in the art and a skilled person will know how to perform them based on common general knowledge. Examples of such assays are disclosed in Materials and Methods of Examples below.

For a given virus, the complement-mediated virus neutralization rate may vary depending on the conditions (e.g.: MOI, culture medium, cell density, temperature, etc., in particular MOI), and when the complement-mediated virus neutralization rates of two viruses are compared, it is in the same conditions. However, similarly to the oncolytic power, any suitable conditions (see examples below) may be used for comparison (provided that they are identical for the two viruses), as the hierarchy of complement-mediated virus neutralization rate will generally remain the same for any suitable conditions when the difference in complement-mediated virus neutralization rate between the two viruses is sufficient. For a given virus, the complement-mediated virus neutralization rate may be generally determined in vitro, in presence of active or heat-inactivated serum with the virus at a dose of 10⁴ to 10⁸ PFU/mL.

The term “treatment” (and any form of treatment such as “treating”, “treat”) as used herein encompasses therapy (e.g.: in a subject diagnosed as having the pathological condition), eventually in association with conventional therapeutic modalities. The result of the treatment is to slow down, cure, ameliorate or control the progression of the targeted pathological condition. For example, a subject is successfully treated for a cancer if after administration of a chimeric poxvirus, a variant chimeric poxvirus, a recombinant chimeric poxvirus or a composition thereof as described herein, alone or in combination, the subject shows an observable improvement of its clinical status.

The term “administering” (or any form of administration, such as “administered”) as used herein refers to the delivery to a subject of a therapeutic agent such as the chimeric poxvirus (variant and/or recombinant) described herein.

As used herein, the term “proliferative disease” encompasses any disease or condition resulting from uncontrolled cell growth and spread including cancers and some cardiovascular diseases (restenosis that results from the proliferation of the smooth muscle cells of the blood vessel wall, etc.). The term “cancer” may be used interchangeably with any of the terms “tumor”, “tumour”, “malignancy”, “neoplasm”, etc. These terms are meant to include any type of tissue, organ or cell, any stage of malignancy (e.g.: from a pre-lesion to stage IV).

The term “subject” generally refers to an organism for whom any product and method of the invention is needed or may be beneficial. Typically, the organism is a mammal, particularly a mammal selected from the group consisting of domestic animals, farm animals, sport animals, and primates. Preferably, the subject is a human who has been diagnosed as having or at risk of having a proliferative disease such as a cancer. The terms “subject” and “patients” may be used interchangeably when referring to a human organism and encompasses male and female. The subject to be treated may be a new-born, an infant, a young adult, an adult or an elderly.

The terms “combination treatment”, “combination therapy”, “combined treatment” or “combinatorial treatment”, may be used interchangeably and refer to a treatment of a subject with a chimeric poxvirus as described herein and at least an additional therapeutic modality. The additional therapeutic modality may be selected from the group consisting of surgery, radiotherapy, chemotherapy, cryotherapy, hormonal therapy, toxin therapy, immunotherapy, cytokine therapy, targeted cancer therapy, gene therapy, photodynamic therapy, transplantation, etc. A combinatorial treatment may include a third or even further therapeutic modality. For combination treatment, it is appreciated that optimal concentration of each component of the combination can be determined by the artisan skilled in the art.

### “Chimeric Poxvirus”, or “Poxvirus Chimera” (Wild-Type)

The present invention relates to chimeric poxviruses with improved properties, similar to the POXSTG19503 chimeric poxvirus generated by the inventors, which displays enhanced oncolytic properties and therapeutic index in vitro and better spreading capacity in vivo due to improved EEV-secretion and syncytia formation capacities, compared to its parental strains.

The terms “chimeric poxvirus” and “poxvirus chimera” are interchangeable and used according to their ordinary meaning in virology: they refer to a hybrid poxvirus created by joining nucleic acid fragments from two or more different poxvirus strains.

The terms “poxvirus”, “poxvirus particle”, “poxvirus vector” and “poxvirus virion” are used interchangeably and are to be understood as meaning a vehicle comprising at least one element of a wild-type poxvirus genome. It is preferred that the poxvirus particle is infectious (i.e.: capable of infecting and entering a host cell or subject). This term encompasses both native as well as genetically modified (e.g.: engineered) poxvirus.

Poxvirus family is characterized by a 200 kb double-stranded DNA genome that encodes numerous viral enzymes and factors that enable the virus to replicate independently from the host cell machinery. The majority of poxviral particles are intracellular (IMV for intracellular mature virion) with a single lipid envelope and remains in the cytosol of infected cells until lysis. The extracellular forms are enveloped particles with an additional membrane that buds out from the infected cell (e.g.: EEV for extracellular enveloped virus).

The nucleic acid sequences of poxviruses are composed by a core sequence and two inverted terminal repeats (ITR). The terms “core”, “core region” or “core sequence” are used interchangeably and designate a nucleic acid region of a poxvirus which is the main viral nucleic acid sequence, flanked by the two ITRs. The length of the core region differs from a poxvirus strain to another. The terms “inverted terminal repeats” or “ITR” designate nucleic acid regions which are duplicated and inverted at both 5′ and 3′ ends of the viral genome. ITRs are composed of non-coding repeated patterns (e.g.: short tandem repeat, microsatellites, minisatellites, etc.) at their extremities that can vary between two viruses. A poxvirus comprises two ITRs, one located on the 5′ end and the other located on the 3′ end of the viral nucleic acid sequence, each one being the reverse complement of the other. The length of the ITRs differs from a poxvirus strain to another.

The term “rabbitpox virus”, “rabbitpox virus particle”, “rabbitpox virus vector” and “rabbitpox virus virion” are used interchangeably. This term encompasses both wild-type, variant, recombinant and recombinant variant rabbitpox viruses.

The term “rabbitpox virus strain Utrecht” (also designated under “RPX” or “RPXV”) is used according to its common, ordinary meaning and refers to virus strains of the same and similar names and functional fragments and homologs thereof. RPX is accessible via culture collections, like ATCC (e.g.: VR-1591™). The term includes wild-type, variant, recombinant and recombinant variant occurring forms of rabbitpox virus strain Utrecht that maintain Utrecht activity. Their genome preferably has sequence identity to the rabbitpox virus strain Utrecht genome (e.g.: about 97%, 98%, 99% or 100%). The genome of RPX used in the examples comprises SEQ ID NO:2, representing the core region (comprised between nucleotide 1 and 183029) and one of the two ITRs (comprised between nucleotide 183030 and 186491). An RPX comprising SEQ ID NO:2 is particularly preferred.

The term “cowpox virus” “cowpox virus particle”, “cowpox virus vector” and “cowpox virus virion” are used interchangeably. This term encompasses both wild-type, variant, recombinant and recombinant variant cowpox viruses.

The term “cowpox virus strain Brighton” (also designated under “CPX” or “CPXV”) is used according to its common, ordinary meaning and refers to virus strains of the same and similar names and functional fragments and homologs thereof. The term includes wild-type, variant, recombinant and recombinant variant occurring forms of cowpox virus strain Brighton or variants thereof that maintain Brighton activity. Their genome preferably has sequence identity to the cowpox virus strain Brighton genome (e.g.: about 97%, 98%, 99% or 100%). The genome of CPX used in the examples comprises SEQ ID NO:3, representing the core region (comprised between nucleotide 1 and 206014) and one of the two ITRs (comprised between nucleotide 206015 and 212521). A CPX comprising SEQ ID NO:3 is particularly preferred.

The terms “vaccinia virus”, “vaccinia virus particle”, “vaccinia virus vector” and “vaccinia virus virion” (also designated under “VACV” or “VV”) are used interchangeably. Vaccinia viruses are accessible via culture collections, like ATCC (e.g.: VR-1354, VR-2056, VR-2034, VR-2035, VR-2010). These terms encompass both wild-type, variant, recombinant and recombinant variant VACV viruses.

The term “Copenhagen” or “COP” is used according to its common, ordinary meaning and refers to virus strains of the same and similar names and functional fragments and homologs thereof. The term includes wild-type, variant, recombinant and recombinant variant occurring forms of vaccinia virus strain COP or variants thereof that maintain COP activity. Their genome preferably has sequence identity to the vaccinia virus strain COP genome (e.g.: about 97%, 98%, 99% or 100%). The genome of COP used in the examples comprises SEQ ID NO:4, representing the core region (comprised between nucleotide 1 and 167702) and one of the two ITRs (comprised between nucleotide 167703 and 175860). A COP comprising SEQ ID NO:4 is particularly preferred.

The term “Wyeth” or “WY” is used according to its common, ordinary meaning and refers to virus strains of the same and similar names and functional fragments and homologs thereof. Said WY is accessible via culture collections, like ATCC (e.g.: VR-1536™). The term includes wild-type, variant, recombinant and recombinant variant occurring forms of vaccinia virus strain Wyeth or variants thereof that maintain Wyeth activity. Their genome preferably has sequence identity to the vaccinia virus strain Wyeth genome (e.g.: about 97%, 98%, 99% or 100%). The genome of WY used in the examples comprises SEQ ID NO:5, representing the core region (comprised between nucleotide 1 and 166358) and one of the two ITRs (comprised between nucleotide 166359 and 182664). A WY comprising SEQ ID NO:5 is particularly preferred.

The term “Western Reserve” or “WR” is used according to its common, ordinary meaning and refers to virus strains of the same and similar names and functional fragments and homologs thereof. Said WR is accessible via culture collections, like ATCC (e.g.: VR-1354™). The term includes wild-type, variant, recombinant and recombinant variant occurring forms of vaccinia virus strain Western Reserve or variants thereof that maintain Western Reserve activity. Their genome preferably has sequence identity to the vaccinia virus strain Western Reserve genome (e.g.: about 97%, 98%, 99% or 100%). The genome of WR used in the examples comprises SEQ ID NO:6, representing the core region (comprised between nucleotide 1 and 174481) and one of the two ITRs (comprised between nucleotide 174482 and 181419). A WR comprising SEQ ID NO:6 is particularly preferred.

The term “Modified Vaccinia Virus” or “MVA” is used according to its common, ordinary meaning and refers to virus strains of the same and similar names and functional fragments and homologs thereof. Said MVA is accessible via culture collections, like ATCC (e.g.: VR-1508™). The term includes wild-type, variant, recombinant and recombinant variant occurring forms of vaccinia virus strain MVA or variants thereof that maintain MVA activity. Their genome preferably has sequence identity to the vaccinia virus strain MVA genome (e.g.: about 97%, 98%, 99% or 100%). The genome of MVA used in the examples comprises SEQ ID NO:7, representing the core region (comprised between nucleotide 1 and 159456) and one of the two ITRs (comprised between nucleotide 159457 and 163444). This vaccinia virus strain MVA genome expresses the eGFP gene under the control of the p11k7.5 promoter (MVATG15938) and was constructed and characterized previously (Erbs et al., 2008, Cancer Gene Ther. 2008, 15, 18-28). An MVA comprising SEQ ID NO:7 is particularly preferred.

The chimeric poxvirus with improved anticancer activity (higher cancer cell killing capacities and better tumor selectivity) may be defined and is defined below by the structure of its genome (nucleic acid features), or by functional features, or as being obtained or obtainable by a specific method of directed evolution as described below, or by combinations of these features.

### Chimeric Poxvirus Defined by Nucleic Acid Features

The core region (comprised between nucleotide 1 and 175910) and one of the two ITRs (comprised between nucleotide 175911 and 185577) of the chimeric poxvirus POXSTG19503 obtained by the inventors have been sequenced and found to correspond to SEQ ID NO:1.

In a first aspect, the invention therefore provides a chimeric poxvirus, wherein said chimeric poxvirus comprises a nucleic acid sequence having a sequence identity of at least 96,6%, preferably at least 96,7%, at least 96,8%, at least 96,9%, at least 97%, at least 97,1%, at least 97,2%, at least 97,3%, at least 97,4%, at least 97,5%, at least 97,6%, at least 97,7%, at least 97,8%, at least 97,9%, at least 98%, at least 98,1%, at least 98,2%, at least 98,3%, at least 98,4%, at least 98,5%, at least 98,6%, at least 98,7%, at least 98,8%, at least 98,9%, at least 99%, at least 99,1%, at least 99,2%, at least 99,3%, at least 99,4%, at least 99,5%, at least 99,6%, at least 99,7%, at least 99,8%, at least 99,9%, at least 99,91%, at least 99,92%, at least 99,93%, at least 99,94%, at least 99,95%, at least 99,96%, at least 99,97%, at least 99,98%, at least 99,99%, or even 100% of identity with SEQ ID NO:1.

In one embodiment, the chimeric poxvirus of the invention is the chimeric poxvirus POXSTG19503 clone 7 deposited at the Collection Nationale de Cultures de Microorganismes (CNCM) on 20 Oct. 2022 under Accession Number CNCM I-5913.

In the present disclosure, the chimeric poxvirus deposited under Accession Number CNCM I-5913 is also referred as POXSTG19503.

The chimeric poxvirus of the invention has preferably been obtained from shuffling of nucleic acid sequences of six parental poxvirus strains: Rabbitpox virus strain Utrecht (RPX), Cowpox virus strain Brighton (CPX), Vaccinia virus strain Copenhagen (COP), Vaccinia virus strain Western Reserve (WR), Vaccinia virus strain Wyeth (WY) and Modified Vaccinia virus strain Ankara (MVA), and more preferably comprises nucleic acid sequences derived from at least two parental poxvirus strains. The nucleic acid fragments from at least two parental poxvirus strains contain the essential genes necessary for replication. The chimeric poxvirus can also comprise nucleic acid sequences derived from at least three, at least four, at least five or even the six parental poxvirus strains.

In particular, the chimeric poxvirus may comprise:
(a) at least one Rabbitpox virus strain Utrecht (RPX)-derived nucleic acid sequence selected from:
   a nucleic acid sequence consisting of nucleotides 562 to 4701 of SEQ ID NO:2 or a sequence with at least 99% identity with nucleotides 562 to 4701 of SEQ ID NO:2,
   a nucleic acid sequence consisting of nucleotides 54042 to 59851 of SEQ ID NO:2 or a sequence with at least 99% identity with nucleotides 54042 to 59851 of SEQ ID NO:2,
   a nucleic acid sequence consisting of nucleotides 83610 to 88879 of SEQ ID NO:2 or a sequence with at least 99% identity with nucleotides 83610 to 88879 of SEQ ID NO:2,
   a nucleic acid sequence consisting of nucleotides 127290 to 130589 of SEQ ID NO:2 or a sequence with at least 99% identity with nucleotides 127290 to 130589 of SEQ ID NO:2,
   a nucleic acid sequence consisting of nucleotides 137520 to 154979 of SEQ ID NO:2 comprising a glutamic acid in position 151 or a sequence with at least 99% identity with nucleotides 137520 to 154979 of SEQ ID NO:2 comprising a glutamic acid in position 151, and
   a nucleic acid sequence consisting of nucleotides 157002 to 162091 of SEQ ID NO:2 or a sequence with at least 99% identity with nucleotides 157002 to 162091 of SEQ ID NO:2;
(b) at least one Cowpox virus strain Brighton (CPX)-derived nucleic acid sequence selected from:
   a nucleic acid sequence consisting of nucleotides 14242 to 51241 of SEQ ID NO:3 or a sequence with at least 99% identity with nucleotides 14242 to 51241 of SEQ ID NO:3, and
   a nucleic acid sequence consisting of nucleotides 59852 to 72141 of SEQ ID NO:3 or a sequence with at least 99% identity with nucleotides 59852 to 72141 of SEQ ID NO:3;
(c) at least one Copenhagen (COP)-derived nucleic acid sequence selected from:
   a nucleic acid sequence consisting of nucleotides 7612 to 8521 of SEQ ID NO:4 or a sequence with at least 99% identity with nucleotides 7612 to 8521 of SEQ ID NO:4;
(d) at least one Wyeth (WY)-derived nucleic acid sequence selected from:
   a nucleic acid sequence consisting of 76630 to 78639 of SEQ ID NO:5 or a sequence with at least 99% identity with nucleotides 76630 to 78639 of SEQ ID NO:5,
   a nucleic acid sequence consisting of 81060 to 83529 of SEQ ID NO:5 or a sequence with at least 99% identity with nucleotides 81060 to 83529 of SEQ ID NO:5,
   a nucleic acid sequence consisting of 116250 to 118459 of SEQ ID NO:5 or a sequence with at least 99% identity with nucleotides 116250 to 118459 of SEQ ID NO:5,
   a nucleic acid sequence consisting of 162290 to 164599 of SEQ ID NO:5 or a sequence with at least 99% identity with nucleotides 162290 to 164599 of SEQ ID NO:5,
   a nucleic acid sequence consisting of 176100 to 179909 of SEQ ID NO:5 or a sequence with at least 99% identity with nucleotides 176100 to 179909 of SEQ ID NO:5, and
   a nucleic acid sequence consisting of 181920 to 184099 of SEQ ID NO:5 or a sequence with at least 99% identity with nucleotides 181920 to 184099 of SEQ ID NO:5;
(e) at least one Western Reserve (WR)-derived nucleic acid sequence selected from:
   a nucleic acid sequence consisting of 169190 to 171579 of SEQ ID NO:6 or a sequence with at least 99% identity with nucleotides 169190 to 171579 of SEQ ID NO:6, and
   a nucleic acid sequence consisting of 173730 to 176099 of SEQ ID NO:6 or a sequence with at least 99% identity with nucleotides 173730 to 176099 of SEQ ID NO:6;
(f) at least one Modified Vaccinia Virus Ankara (MVA)-derived nucleic acid sequence selected from:
   a nucleic acid sequence consisting of 88880 to 90899 of SEQ ID NO:7 or a sequence with at least 99% identity with nucleotides 88880 to 90899 of SEQ ID NO:7,
   a nucleic acid sequence consisting of 91460 to 93839 of SEQ ID NO:7 or a sequence with at least 99% identity with nucleotides 91460 to 93839 of SEQ ID NO:7,
   a nucleic acid sequence consisting of 95530 to 116249 of SEQ ID NO:7 or a sequence with at least 99% identity with nucleotides 95530 to 116249 of SEQ ID NO:7,
   a nucleic acid sequence consisting of 118460 to 127289 of SEQ ID NO:7 or a sequence with at least 99% identity with nucleotides 118460 to 127289 of SEQ ID NO:7, and
   a nucleic acid sequence consisting of 134800 to 137519 of SEQ ID NO:7 or a sequence with at least 99% identity with nucleotides 134800 to 137519 of SEQ ID NO:7;
   or
(g) any combination of (a) to (f).

In a preferred embodiment of the invention, the chimeric poxviruses comprise a glutamic acid in position 151 (151E) of the protein encoded by the A34R gene. Said amino acid in position 151 in A34R gene is known to be linked with increased production of total progeny virus and extracellular enveloped virus (EEV), a form that can be immune-evasive and with enhanced spreading (Thirunavukarasu et al., 2013, Mol. Ther. Vol 21 no.5, p.1024-1033). The sequence containing the glutamic acid in position 151 of the protein encoded by the A34R gene may be inherited from Rabbitpox virus, preferably from the parental Rabbitpox virus strain Utrecht (RPX). In a further preferred embodiment of the invention, the chimeric poxviruses comprise a valine in position 19 (19V) of the protein encoded by the A34R gene. The sequence containing the valine in position 19 of the protein encoded by the A34R gene may be inherited from the parental Modified Vaccinia Virus Ankara (MVA). In a more preferred embodiment of the invention, the chimeric poxviruses comprise a A34R chimeric gene wherein said A34R chimeric gene is encoding a protein comprising a valine in position 19 (19V) and a glutamic acid in position 151 (151E). The sequence containing the valine in position 19 (19V) and the glutamic acid in position 151 (151E) of the protein encoded by the A34R gene may be inherited from the parental Modified Vaccinia Virus Ankara (MVA) and the parental Rabbitpox virus strain Utrecht (RPX), respectively. In an even more preferred embodiment, the protein encoded by the A34R chimeric gene has at least 85%, preferably at least 90%, and more preferably at least 95%, or has 100% identity with the amino acid sequence of SEQ ID NO:11 and a glutamic acid position in position 151 (151E), and optionally a valine in position 19 (19V).

In a further preferred embodiment of the invention, the chimeric poxviruses are partially or totally defective in A56R locus. Said deficiency in A56R locus resulting in alteration of the Hemagglutinin encoding-gene is linked with induced syncytia formation in infected cells by avoiding the inhibition of the cell-cell fusion (Turner et al. 2008, Virology 380, 226-233). The said chimeric poxviruses are partially or totally defective in A56R locus, resulting in either: the expression of a non-effective protein encoded by the A56R gene, deletion or mutation of the N-terminus domain (for instance at least 34 amino acids, preferably at least 36 amino acids, at least 38 amino acids, more preferably at least 40 amino acids, at least 42 amino acids, or at least 44 amino acids) of the protein encoded by the A56R gene, mutation in position 34 and/or 103 of the protein encoded by the A56R gene compared to the one expressed by the parental Vaccinia virus strain Copenhagen (COP) sequence. In particular, the chimeric poxviruses may comprise a 44 amino acid residues deletion in N-Terminus domain of the protein expressed by the A56R gene compared to the one expressed by the parental Vaccinia virus strain Copenhagen (COP) sequence. In a more particular embodiment, the chimeric poxviruses may comprise the sequence of the protein encoded by the A56R gene inherited from Rabbitpox virus, preferably from the parental Rabbitpox virus strain Utrecht (RPX). In even more preferred embodiment, the protein encoded by the A56R gene has at least 85%, preferably at least 90%, and more preferably at least 95%, or has 100% identity with the amino acid sequence shown in SEQ ID NO:12.

Alternatively, or in combination, the above-described chimeric poxviruses according to the invention may comprise a gene encoding a zinc RING finger protein, wherein said gene may be inherited from Rabbitpox virus, preferably from the parental Rabbitpox virus strain Utrecht (RPX).

Alternatively, or in combination, the above-described chimeric poxviruses according to the invention preferably do not comprise the gene inherited from Rabbitpox virus, preferably from the parental Rabbitpox virus strain Utrecht (RPX), encoding the ankyrin repeat protein.

Alternatively, or in combination, the above-described chimeric poxviruses according to the invention may preferably do not comprise the gene inherited from Rabbitpox virus, preferably from the parental Rabbitpox virus strain Utrecht (RPX), encoding the chemokine-binding protein.

Alternatively, or in combination, the above-described chimeric poxviruses according to the invention may preferably:
comprise a gene encoding a zinc RING finger protein, wherein said gene may be inherited from Rabbitpox virus, preferably from the parental Rabbitpox virus strain Utrecht (RPX), and
do not comprise the genes inherited from Rabbitpox virus, preferably from the parental Rabbitpox virus strain Utrecht (RPX), encoding the ankyrin repeat protein and the chemokine-binding protein.

### Chimeric Poxvirus Defined by Functional Features

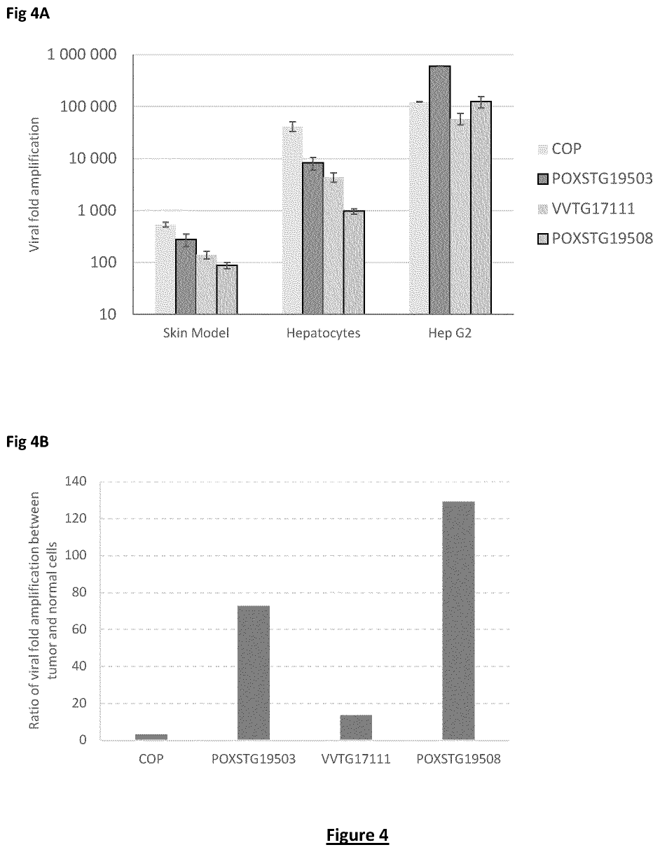

The chimeric poxvirus POXSTG19503 obtained by the inventors is characterized by several advantageous functional properties, including higher oncolytic power and replication than parental strain COP in several tumor cells (see FIGS. 1 and 4A) and lower replication in healthy cells (preferably primary cells) (see FIG. 4A) and thus higher therapeutic index (see FIG. 4B) than parental strain COP.

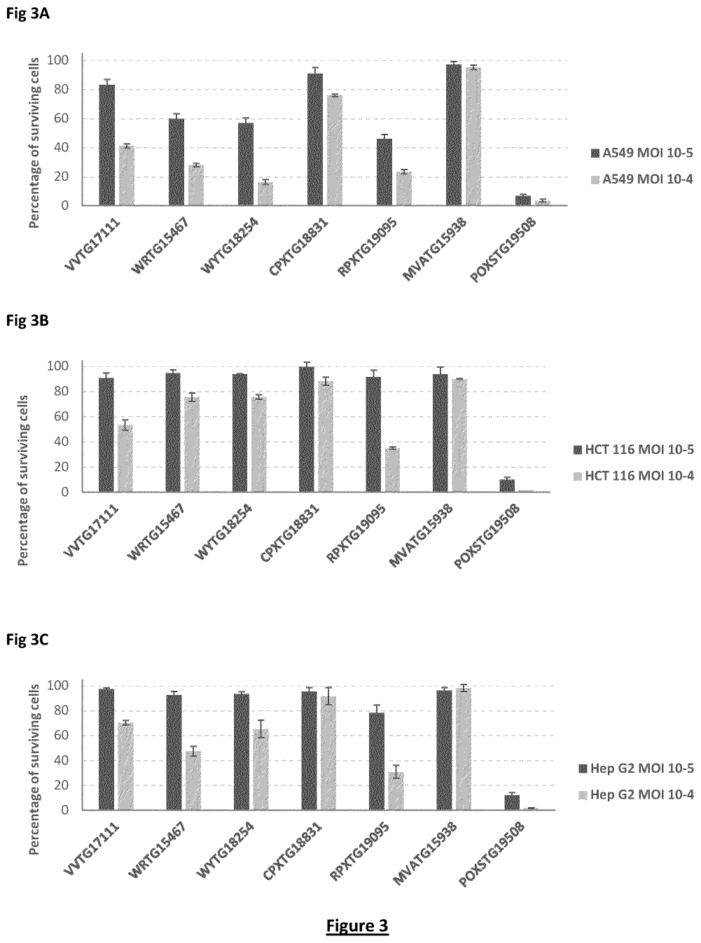

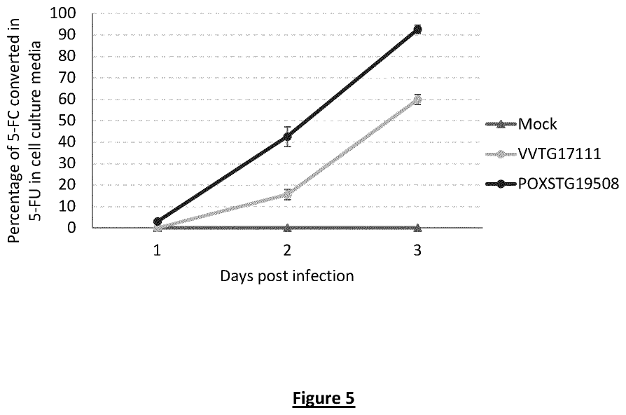

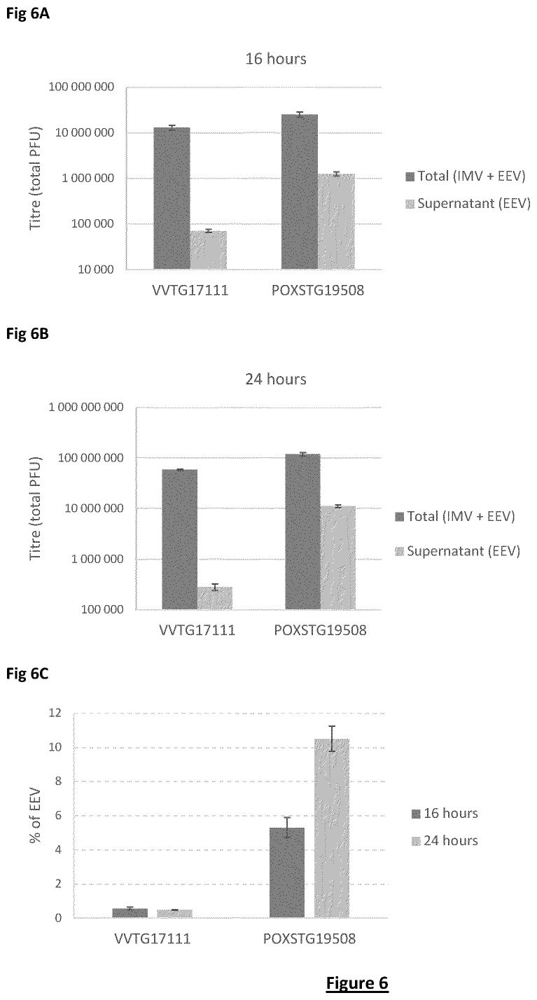

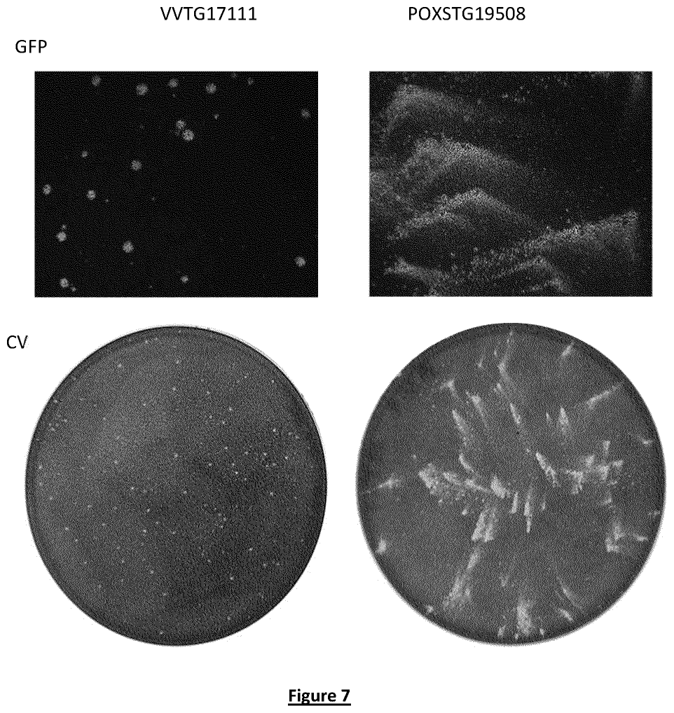

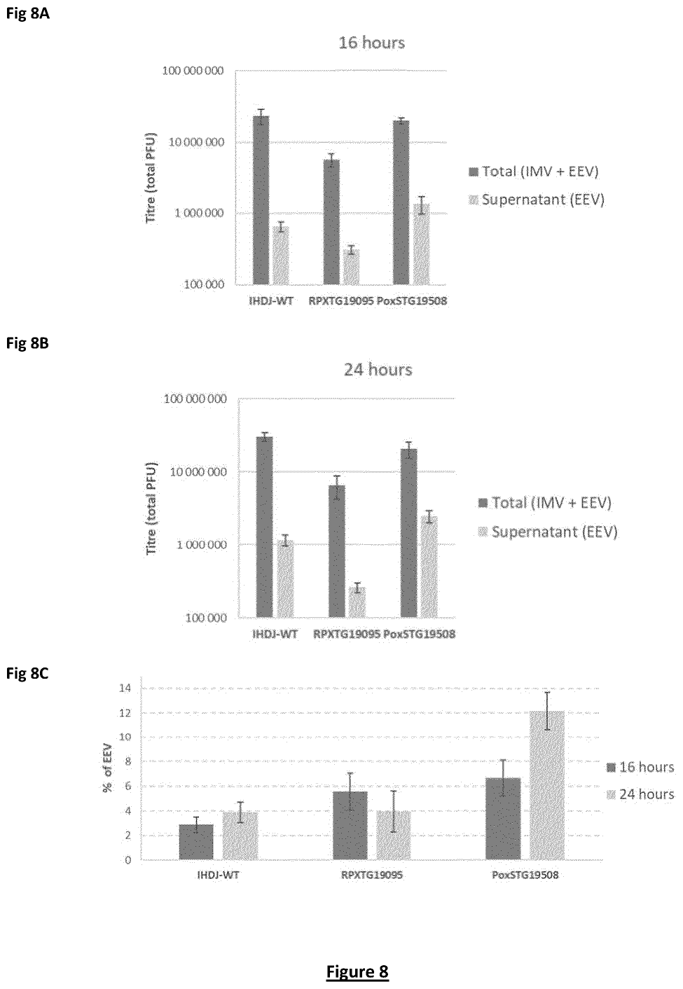

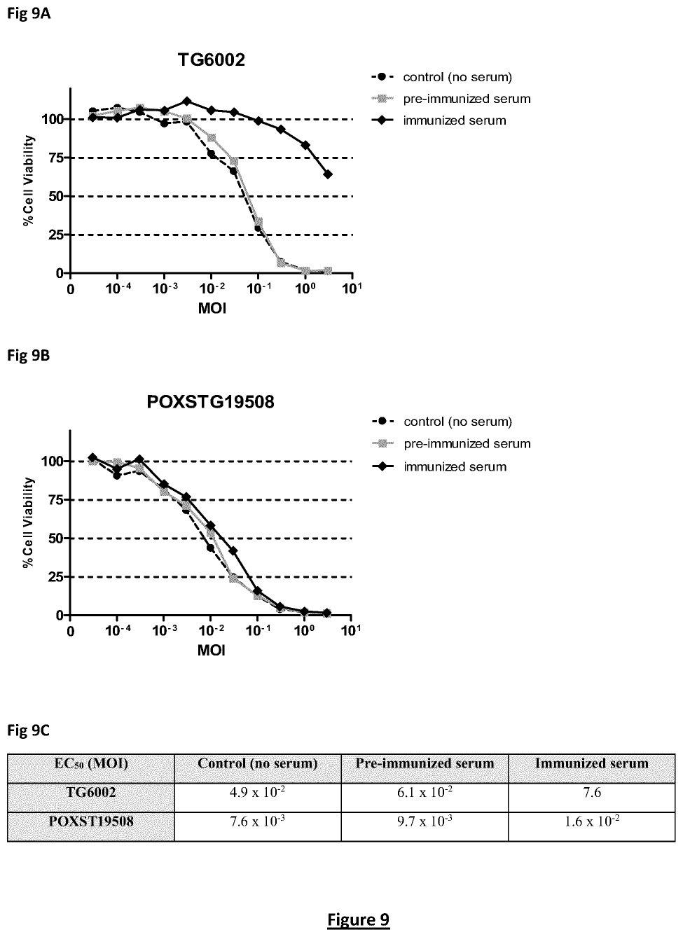

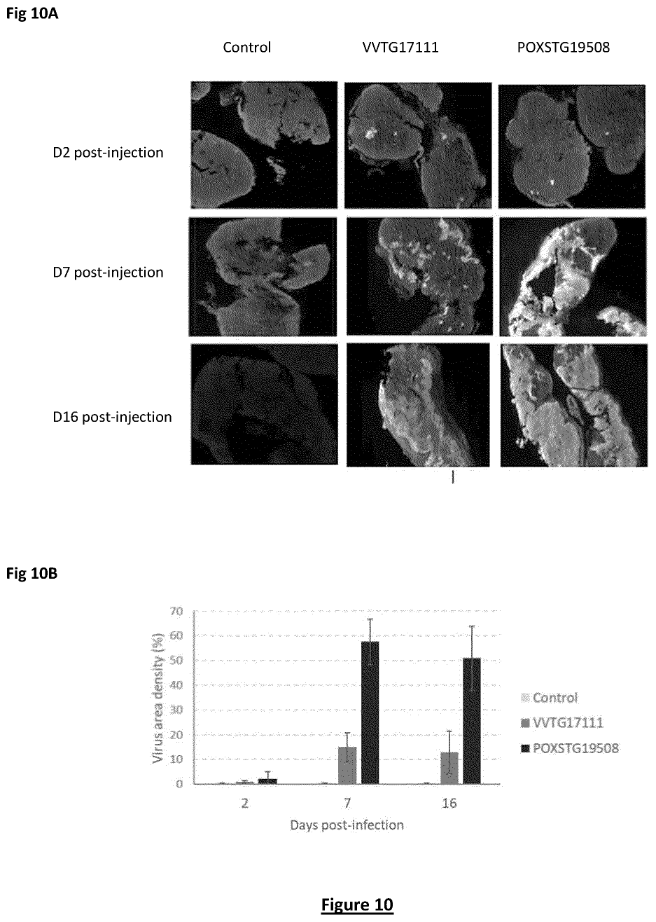

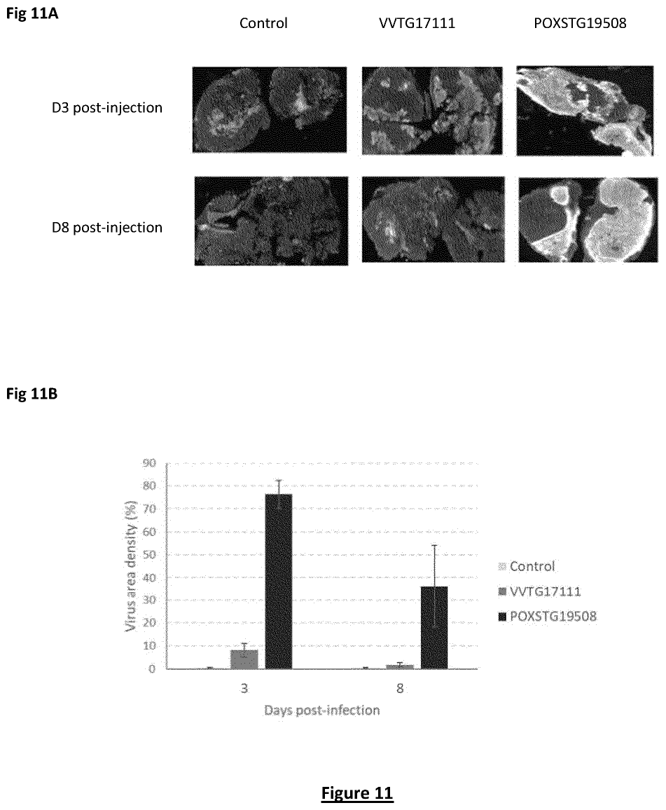

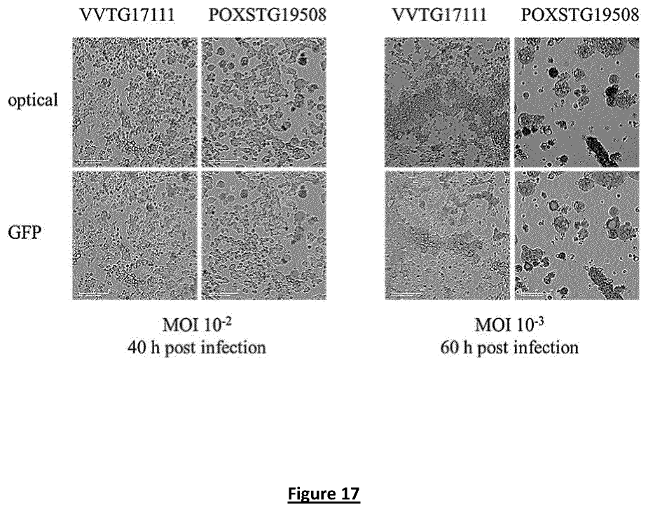

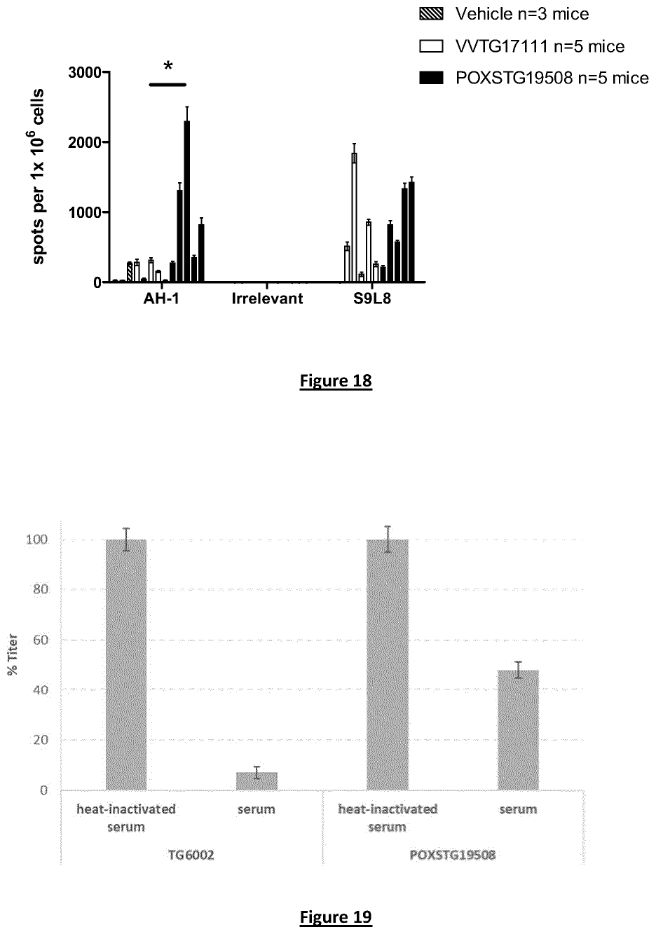

In addition, a TK-variant thereof (POXSTG19508) has been found to display higher oncolytic power than TK-variants of all parental strains (see FIG. 3), higher replication in cancer cells and lower replication in healthy cells (preferably primary cells) and thus higher therapeutic index than TK-COP parental strain (see FIG. 4); expresses higher amounts of transgene than TK-COP parental strain (see FIG. 5), produces higher ratios of EEV than TK-COP parental strain (see FIG. 6), RPX and IHDJ-WT (see FIG. 8), induces syncytia formation (see FIG. 17), resulting in higher number and size of tails in comet assay (see FIG. 7) and better spreading in vivo (see FIG. 10). The TK-variant POXSTG19508 was also found to be less sensitive to poxvirus-specific antibody neutralization (see FIG. 9), less sensitive to complement-mediated virus neutralization (see FIG. 19), more efficient in various animal models in vivo (see FIGS. 11-15) and induced a superior T cell response against tumor (see FIG. 18).

Similar results have been obtained with a TK-RR-variant (POXSTG19730, data not shown).

Each of these improved functional features is of interest for improved oncolytic treatments of proliferative diseases, in particular cancer.

Chimeric Poxvirus with High Oncolytic Power

Therefore, in another aspect, the invention provides a chimeric poxvirus (optionally variant and/or recombinant), wherein, for at least one tumor, the oncolytic power of said chimeric poxvirus is higher than the oncolytic power measured in the same conditions and at the same time post-infection of at least one of the oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR), wherein for a given tumor, a given virus, given conditions and a given time post-infection, an oncolytic power OP(tumor, virus, conditions, time post-infection) is defined as:
OP(tumor, virus, conditions, time post-infection)=(100−percentage of surviving tumor cells after infection by virus).

In a preferred embodiment, for at least one tumor, the oncolytic power of said chimeric poxvirus is higher than the oncolytic power measured in the same conditions and at the same time post-infection of the parental COP or the parental RPX. More preferably, for at least one tumor, the oncolytic power of said chimeric poxvirus is higher than the oncolytic power measured in the same conditions and at the same time post-infection of at least two of the oncolytic parental poxvirus strains RPX, CPX, COP, WY and WR. For instance, for at least one tumor, the oncolytic power of said chimeric poxvirus is higher than the oncolytic powers measured in the same conditions and at the same time post-infection of the parental COP and the parental CPX. In a more preferred embodiment, for at least one tumor, the oncolytic power of said chimeric poxvirus is higher than the oncolytic power measured in the same conditions and at the same time post-infection of at least three, more preferably higher than at least four, and even more preferably higher than each of the five oncolytic parental poxvirus strains RPX, CPX, COP, WY and WR.

For a given tumor and virus, the oncolytic power may generally be determined in vitro, 3 to 5 days post-infection of tumor cells with the virus at an MOI of 10⁻⁵ to 10⁻².

In a specific embodiment of this aspect, the invention provides a chimeric poxvirus (optionally variant and/or recombinant), wherein, for at least one tumor cell line selected from A549, MIA Paca-2, U-87-MG, B16F10 and HepG2, the oncolytic power of said chimeric poxvirus is higher than the oncolytic power measured in the same conditions and at the same time post-infection of at least one of the oncolytic parental poxvirus strains RPX, CPX, COP, WY and WR. In a preferred embodiment, for at least one tumor cell line selected from A549, MIA Paca-2, U-87-MG, B16F10 and HepG2, the oncolytic power of said chimeric poxvirus is higher than the oncolytic power measured in the same conditions and at the same time post-infection of the parental COP or RPX. More preferably, for at least one tumor cell line selected from A549, MIA Paca-2, U-87-MG, B16F10 and HepG2, the oncolytic power of said chimeric poxvirus is higher than the oncolytic power measured in the same conditions and at the same time post-infection of at least two of the oncolytic parental poxvirus strains RPX, CPX, COP, WY and WR. For instance, for at least one tumor cell line selected from A549, MIA Paca-2, U-87-MG, B16F10 and HepG2, the oncolytic power of said chimeric poxvirus is higher than the oncolytic powers measured in the same conditions and at the same time post-infection of the parental COP and the parental RPX. In a more preferred embodiment, for at least one tumor cell line selected from A549, MIA Paca-2, U-87-MG, B16F10 and HepG2, the oncolytic power of said chimeric poxvirus is higher than the oncolytic power measured in the same conditions and at the same time post-infection of at least three, more preferably higher than at least four, and even more preferably higher than each of the five oncolytic parental poxvirus strains RPX, CPX, COP, WY and WR.

In a more specific embodiment of this aspect,
a) on A549, the difference between the oncolytic power of the chimeric poxvirus and the oncolytic power measured in the same conditions and at the same time post-infection of at least one of the oncolytic parental poxvirus strains RPX, CPX, COP, WY and WR is at least 69%. Preferably, the difference between the oncolytic power of said chimeric poxvirus and the oncolytic power measured in the same conditions and at the same time post-infection of a parental COP(defective in the J2R locus or not) or a parental RPX is at least 69%. More preferably, the difference between the oncolytic power of said chimeric poxvirus and the oncolytic power measured in the same conditions and at the same time post-infection of at least two of the oncolytic parental poxvirus strains RPX, CPX, COP, WY and WR is at least 69%. For instance, the difference between the oncolytic power of said chimeric poxvirus and the oncolytic power measured in the same conditions and at the same time post-infection of a parental COP and a parental RPX is at least 69%. In a more preferred embodiment, the difference between the oncolytic power of said chimeric poxvirus and the oncolytic power measured in the same conditions and at the same time post-infection of at least three, more preferably at least four, and even more preferably all five of the parental oncolytic poxvirus strains RPX, CPX, COP, WY and WR is at least 69%; and/or
b) on MIA PaCa-2, the difference between the oncolytic power of the chimeric poxvirus and the oncolytic power measured in the same conditions and at the same time post-infection of at least one of the oncolytic parental poxvirus strains RPX, CPX, COP, WY and WR is at least 37%. Preferably, the difference between the oncolytic power of said chimeric poxvirus and the oncolytic power measured in the same conditions and at the same time post-infection of a parental COP(defective in the J2R locus or not) or a parental RPX is at least 37%. More preferably, the difference between the oncolytic power of said chimeric poxvirus and the oncolytic power measured in the same conditions and at the same time post-infection of at least two of the oncolytic parental poxvirus strains RPX, CPX, COP, WY and WR is at least 37%. For instance, the difference between the oncolytic power of said chimeric poxvirus and the oncolytic power measured in the same conditions and at the same time post-infection of a parental COP and a parental RPX is at least 37%. In a more preferred embodiment, the difference between the oncolytic power of said chimeric poxvirus and the oncolytic power measured in the same conditions and at the same time post-infection of at least three, more preferably at least four, and even more preferably all five of the parental oncolytic poxvirus strains RPX, CPX, COP, WY and WR is at least 37%; and/or
c) on U-87 MG, the difference between the oncolytic power of the chimeric poxvirus and the oncolytic power measured in the same conditions and at the same time post-infection of at least one of the oncolytic parental poxvirus strains RPX, CPX, COP, WY and WR is at least 37%. Preferably, the difference between the oncolytic power of said chimeric poxvirus and the oncolytic power measured in the same conditions and at the same time post-infection of a parental COP or a parental RPX is at least 37%. More preferably, the difference between the oncolytic power of said chimeric poxvirus and the oncolytic power measured in the same conditions and at the same time post-infection of at least two of the oncolytic parental poxvirus strains RPX, CPX, COP, WY and WR is at least 37%. For instance, the difference between the oncolytic power of said chimeric poxvirus and the oncolytic power measured in the same conditions and at the same time post-infection of a parental COP and a parental RPX is at least 37%. In a more preferred embodiment, the difference between the oncolytic power of said chimeric poxvirus and the oncolytic power measured in the same conditions and at the same time post-infection of at least three, more preferably at least four, and even more preferably all five of the parental oncolytic poxvirus strains RPX, CPX, COP, WY and WR is at least 37%; and/or
d) on B16F10, the difference between the oncolytic power of the chimeric poxvirus and the oncolytic power measured in the same conditions and at the same time post-infection of at least one of the oncolytic parental poxvirus strains RPX, CPX, COP, WY and WR is at least 20%. Preferably, the difference between the oncolytic power of said chimeric poxvirus and the oncolytic power measured in the same conditions and at the same time post-infection of a parental COP or a parental RPX is at least 20%. More preferably, the difference between the oncolytic power of said chimeric poxvirus and the oncolytic power measured in the same conditions and at the same time post-infection of at least two of the oncolytic parental poxvirus strains RPX, CPX, COP, WY and WR is at least 20%. For instance, the difference between the oncolytic power of said chimeric poxvirus and the oncolytic power measured in the same conditions and at the same time post-infection of a parental COP and a parental RPX is at least 20%. In a more preferred embodiment, the difference between the oncolytic power of said chimeric poxvirus and the oncolytic power measured in the same conditions and at the same time post-infection of at least three, more preferably at least four, and even more preferably all five of the parental oncolytic poxvirus strains RPX, CPX, COP, WY and WR is at least 20%; and/or
e) on HepG2, the difference between the oncolytic power of the chimeric poxvirus and the oncolytic power measured in the same conditions and at the same time post-infection of at least one of the oncolytic parental poxvirus strains RPX, CPX, COP, WY and WR is at least 49%. Preferably, the difference between the oncolytic power of said chimeric poxvirus and the oncolytic power measured in the same conditions and at the same time post-infection of a parental COP or a parental RPX is at least 49%. More preferably, the difference between the oncolytic power of said chimeric poxvirus and the oncolytic power measured in the same conditions and at the same time post-infection of at least two of the oncolytic parental poxvirus strains RPX, CPX, COP, WY and WR is at least 49%. For instance, the difference between the oncolytic power of said chimeric poxvirus and the oncolytic power measured in the same conditions and at the same time post-infection of a parental COP and a parental RPX is at least 49%. In a more preferred embodiment, the difference between the oncolytic power of said chimeric poxvirus and the oncolytic power measured in the same conditions and at the same time post-infection of at least three, more preferably at least four, and even more preferably all five of the parental oncolytic poxvirus strains RPX, CPX, COP, WY and WR is at least 49%.

Preferably, in this embodiment, the oncolytic power is evaluated 5 days post infection and at a MOI of 10⁵ on A549, U-87-MG and HepG2, at an MOI of 10⁴ on MIA PaCa-2, and at a MOI of 10⁻³ on B16F10. More preferably, in this embodiment, the tumor cell lines are cultured at 37° C., 5% CO₂, in Dulbecco's Modified Eagle Medium (DMEM) with 10% of Fetal Calf Serum (FCS).

Chimeric Poxvirus with Low Replication in Healthy Cells (Preferably Primary Cells)

In another aspect, the invention provides a chimeric poxvirus (optionally variant and/or recombinant), wherein for at least one healthy cell (preferably primary cell), the viral replication in the healthy cell of said chimeric poxvirus is lower than that of at least one of the five oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR). Preferably, for at least one healthy cell (preferably primary cell), the viral replication in the healthy cell of said chimeric poxvirus is lower than that of the parental Vaccinia virus strain Copenhagen (COP). More preferably, for at least one healthy cell (preferably primary cell), the viral replication in the healthy cell (preferably primary cell) of said chimeric poxvirus is lower than that of at least two, preferably at least three, more preferably at least four, and even more preferably each of the five oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR).

For a given tumor or healthy cell (preferably primary cell), the viral replication may generally be determined in vitro, 2 to 8 days post-infection of tumor or healthy cells (preferably primary cells) with the virus at an MOI of 10⁻⁵ to 10⁻².

In a specific embodiment of this aspect, the invention provides a chimeric poxvirus (optionally variant and/or recombinant), wherein for at least one healthy cell (preferably primary cell) selected from skin cells and hepatocytes, the viral replication in the healthy cell of said chimeric poxvirus is lower than that of at least one of the five oncolytic parental poxvirus strains RPX, CPX, COP, WY and WR. Preferably, for at least one healthy cell (preferably primary cell) selected from skin cells and hepatocytes, the viral replication in the healthy cell of said chimeric poxvirus is lower than that of the parental COP. More preferably, for at least one healthy cell (preferably primary cell) selected from skin cells and hepatocytes, the viral replication in the healthy cell (preferably primary cell) of said chimeric poxvirus is lower than that of at least two, preferably at least three, more preferably at least four, and even more preferably each of the five oncolytic parental poxvirus strains RPX, CPX, COP, WY and WR.

In a more specific embodiment of this aspect, the viral replication of said chimeric poxvirus is:
a) at least 1.9 times lower in at least one healthy cell line (preferably primary cell line) selected from skin cells and hepatocytes than that of at least one of the five oncolytic parental poxvirus strains (optionally variant and/or recombinant) RPX, CPX, COP, WY and WR, preferably than that of the parental COP, more preferably than that of at least two, more preferably at least three, more preferably at least four, and even more preferably each of the five oncolytic parental poxvirus strains (optionally variant and/or recombinant) RPX, CPX, COP, WY and WR; and/or
b) at least 5.1 times lower in hepatocytes than that of at least one of the five oncolytic parental poxvirus strains (optionally variant and/or recombinant) RPX, CPX, COP, WY and WR, preferably than that of the parental COP, more preferably than that of at least two, more preferably at least three, more preferably at least four, and even more preferably each of the five oncolytic parental poxvirus strains (optionally variant and/or recombinant) RPX, CPX, COP, WY and WR.

Preferably, in this embodiment, the viral replication is evaluated in vitro 3 days post infection and at a MOI of 10⁻⁴ on HepG2, or 7 days post infection and at 10⁵ PFU on human skin model. More preferably, in this embodiment, the tumor cell lines are cultured at 37° C., 5% CO₂, in Dulbecco's Modified Eagle Medium (DMEM) with 10% of Fetal Calf Serum (FCS).

Chimeric Poxvirus with High Therapeutic Index

In another aspect, the invention provides a chimeric poxvirus (optionally variant and/or recombinant), wherein, for at least one organ, the therapeutic index of said chimeric poxvirus is higher than the therapeutic index measured in the same conditions and at the same time post-infection of at least one of the five oncolytic parental poxvirus strain(s) Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) wherein for a given organ, a given tumor, a given virus, given conditions and a given time post-infection, the therapeutic index TI(organ, tumor, virus, conditions, time post-infection) is defined as:
TI(organ, tumor, virus, conditions, time post-infection)=(replication of virus in organ tumor cells/replication of virus in organ healthy cells).

In a preferred embodiment, for at least one organ, the therapeutic index of said chimeric poxvirus is higher than the therapeutic index measured in the same conditions and at the same time post-infection of the parental Vaccinia virus strain Copenhagen (COP). More preferably, for at least one organ, the therapeutic index of said chimeric poxvirus is higher than the therapeutic index measured in the same conditions and at the same time post-infection of at least two of, more preferably at least three of, more preferably at least four of, and even more preferably each of the five oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR).

For a given organ and virus, the therapeutic index may generally be determined in vitro, 2 to 8 days post-infection of tumor or healthy cells (preferably primary cells) with the virus at an MOI of 10⁵ to 10⁻².

The therapeutic index is related to both the oncolytic power and the replication in healthy cells (preferably primary cells). A higher therapeutic index may be due to higher oncolytic power, lower replications in in healthy cells (preferably primary cells) or both.

In a specific embodiment of this aspect, the hepatic therapeutic index of said chimeric poxvirus (optionally variant and/or recombinant) is higher than the hepatic therapeutic index measured in the same conditions and at the same time post-infection of at least one of the parental oncolytic poxvirus strains RPX, CPX, COP, WY and WR (optionally variant and/or recombinant), wherein for a given virus, an hepatic therapeutic index TI(liver, HepG2, virus, conditions, time post-infection) is defined as:
TI(liver, HepG2, virus, conditions, time post-infection)=(replication of virus in HepG2 tumor cells/replication of virus in healthy hepatocytes).

Preferably, the hepatic therapeutic index of said chimeric poxvirus (optionally variant and/or recombinant) is higher than the hepatic therapeutic index measured in the same conditions and at the same time post-infection of the parental COP. In a more preferred embodiment, the hepatic therapeutic index of said chimeric poxvirus is higher than the hepatic therapeutic index measured in the same conditions and at the same time post-infection of at least two, preferably at least three, more preferably at least four, and even more preferably all five of the parental oncolytic poxvirus strains RPX, CPX, COP, WY and WR.

Preferably, the hepatic therapeutic index of said chimeric poxvirus is at least 5 times higher than, more preferably at least 10 times higher than, more preferably at least 15 times higher than, even more preferably at least 20 times higher than the hepatic therapeutic index measured in the same conditions and at the same time post-infection of at least one of the parental oncolytic poxvirus strains RPX, CPX, COP, WY and WR (optionally variant and/or recombinant), preferably than the hepatic therapeutic index measured in the same conditions and at the same time post-infection of the parental COP, more preferably higher than the hepatic therapeutic index measured in the same conditions and at the same time post-infection of at least two, preferably at least three, more preferably at least four, and even more preferably all five of the parental oncolytic poxvirus strains RPX, CPX, COP, WY and WR.

More preferably, in this embodiment, the hepatic therapeutic index is measured in vitro in a setting in which the chimeric poxvirus is added respectively to the HepG2 tumor cells at a MOI of 10⁻⁵ and to the healthy hepatocytes (preferably primary hepatocytes) at a MOI of 10⁻⁴, and the replication of the chimeric poxvirus in the HepG2 tumor cells and in the healthy hepatocytes is measured 3 days post infection. Even more preferably, in this embodiment, the HepG2 tumor cell lines are cultured at 37° C., 5% CO₂, in Dulbecco's Modified Eagle Medium (DMEM) with 10% of Fetal Calf Serum (FCS), and the healthy hepatocytes are cultured at 37° C., 5% CO₂, in basal hepatic cell medium (BIOPREDICS catalogue reference MIL600) and additives for hepatocytes culture medium (BIOPREDICS catalogue reference ADD222C).

Chimeric Poxvirus with High EEV-Secretion Capacity

Poxviruses have two distinct infectious virus particles which can initiate infection: the intracellular mature virus (IMV) and the extracellular enveloped virus (EEV). The EEV is typically produced early after infection of a susceptible cell and is released from the cell before lysis. Therefore, the EEV form spreads rapidly and systemically within the infected host and evades immune-mediated clearance from the blood. Kirn et al. (2008, Cancer Res. 68 (7): 2071-5) compared the oncolytic potential of low versus high EEV-producing strains of vaccinia. Significantly improved antitumor effects were observed with EEV-enhanced vaccinia strains, which displayed improved spreading within tumors after systemic delivery. Moreover, the EEV-enhanced strains also displayed a greater ability to spread between injected and noninjected distant tumors through the blood. Importantly, EEV-enhanced strains also displayed reduced clearance by circulating neutralizing antibody.

In another aspect, the invention provides a chimeric poxvirus (optionally variant and/or recombinant), wherein for at least one producer cell (preferably a tumor cell), the extracellular-enveloped virus (EEV)-secretion capacity (SC) (abbreviated as EEV-SC) of said chimeric poxvirus is higher than the EEV-SC measured in the same conditions and at the same time post-infection of at least one of the six parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA), wherein for a given virus, a given producer cell, given conditions and a given time after viral infection, said EEV-SC is the ratio of extracellular enveloped virus (EEV) to the total forms of the virus (extracellular enveloped virus (EEV) and intracellular mature virus (IMV) form of the virus), defined as:
EEV-SC (virus, producer cell, conditions, time post-infection)=number of EEV particles/number of (EEV+IMV) particles.

In a preferred embodiment, the invention provides a chimeric poxvirus (optionally variant and/or recombinant), wherein for at least one producer cell (preferably a tumor cell), the EEV-SC of said chimeric poxvirus is higher than the EEV-SC measured in the same conditions and at the same time post-infection of the parental Vaccinia virus strain Copenhagen (COP) or the parental Rabbitpox virus strain Utrecht (RPX). More preferably, for at least one producer cell (preferably a tumor cell), the EEV-SC of said chimeric poxvirus is higher than the EEV-SC measured in the same conditions and at the same time post-infection of at least two of the six parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA). For instance, for at least one producer cell (preferably a tumor cell), the EEV-SC of said chimeric poxvirus is higher than the EEV-SCs measured in the same conditions and at the same time post-infection of the parental Vaccinia virus strain Copenhagen (COP) and the parental Rabbitpox virus strain Utrecht (RPX). In a more preferred embodiment, for at least one producer cell (preferably a tumor cell), the EEV-SC of said chimeric poxvirus is higher than the EEV-SC measured in the same conditions and at the same time post-infection of at least three, preferably at least four, more preferably at least five, and even more preferably each of the six parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA).

Vaccinia virus strain IHD-J is known as a high EEV producer strain of vaccinia virus. Therefore, alternatively, or in combination, for at least one producer cell (preferably a tumor cell), the EEV-SC of said chimeric poxvirus (optionally variant and/or recombinant) is higher than the EEV-SC measured in the same conditions and at the same time post-infection of Vaccinia virus strain IHD-J. For a given tumor and virus, the EEV-SC may generally be determined in vitro, 16 to 24 hours post-infection of tumor cells with the virus at an MOI of 10⁻⁴ to 10⁻¹.

In a more specific embodiment of this aspect, the EEV-SC of the chimeric poxvirus is:
a) at least 4%, preferably at least 5%, more preferably at least 6%, 16 hours post-infection of the said chimeric poxvirus on A549 tumor cell line, at an MOI of 10⁻² to 1, preferably at an MOI of 0.1; and/or
b) at least 5%, at least 6%, at least 7%, at least 8%, preferably at least 9%, more preferably at least 10%, even more preferably at least 11%, 24 hours post-infection of the said chimeric poxvirus on A549 tumor cell line, at an MOI of 10⁻² to 1, preferably at an MOI of 0.1.

In a more specific embodiment of this aspect, the EEV-SC of the chimeric poxvirus is:
a) comprised between 4% and 9%, more preferably between 6% and 8%, 16 hours post-infection of the said chimeric poxvirus on A549 tumor cell line at an MOI of 10⁻² to 1, preferably at an MOI of 0.1; and/or
b) comprised between 5% and 15%, more preferably between 10% and 13%, 24 hours post-infection of the said chimeric poxvirus on A549 tumor cell line at an MOI of 10⁻² to 1, preferably at an MOI of 0.1.
   Chimeric Poxvirus with High Spreading Capacity

Preferably, for at least one tumor, the spreading capacity of said chimeric poxvirus (optionally variant and/or recombinant) is higher than the spreading capacity measured in the same conditions and at the same time post-infection of at least one of the six parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA), wherein for a given virus, producer cell, conditions and time post-infection, the spreading capacity is defined as the capacity of the virus to disseminate between cells (e.g. tumor cells) or between tumors (e.g. spreading from an injected tumor to a distal tumor). In a preferred embodiment, for at least one tumor, the spreading capacity of said chimeric poxvirus is higher than the spreading capacity measured in the same conditions and at the same time post-infection of the parental COP or the parental RPX. More preferably, for at least one tumor, the spreading capacity of said chimeric poxvirus is higher than the spreading capacity measured in the same conditions and at the same time post-infection of at least two of the six parental poxvirus strains RPX, CPX, COP, WY, WR and MVA. For instance, for at least one tumor, the spreading capacity of said chimeric poxvirus is higher than the spreading capacities measured in the same conditions and at the same time post-infection of the parental COP and the parental RPX. In a more preferred embodiment, for at least one tumor, the spreading capacity of said chimeric poxvirus is higher than the spreading capacity of at least three, preferably at least four, more preferably at least five or even more preferably each of the six parental poxvirus strains RPX, CPX, COP, WY, WR and MVA.

For a given tumor and virus, the spreading capacity may generally be determined in vitro, 16 to 24 hours post-infection of tumor cells with the virus at an MOI of 10⁻⁴ to 10⁻¹.

Chimeric Poxvirus with Low Neutralization Rate in the Presence of Poxvirus-Specific Antibody

In another aspect, the invention provides a chimeric poxvirus (optionally variant and/or recombinant), wherein, for at least one poxvirus-specific antibody and tumor, the neutralization rate of said chimeric poxvirus is lower than the neutralization rate measured in the same conditions and at the same time post-infection of at least one of the six parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA), wherein for a given virus, a given tumor, a given poxvirus-specific antibody, given conditions and a given time post-infection, the neutralization rate (NT(virus, tumor, conditions, time post-infection)) measures the antibody-induced inhibition of the virus' oncolytic power and is defined as:
NT(virus, tumor, Poxvirus-specific antibody, conditions, time post-infection)=EC50 (with poxvirus-specific antibody)/EC50 (without poxvirus-specific antibody).

In a preferred embodiment, for at least one poxvirus-specific antibody and tumor, the neutralization rate of said chimeric poxvirus is lower than the neutralization rate measured in the same conditions and at the same time post-infection of the parental COP. More preferably, for at least one poxvirus-specific antibody and tumor, the neutralization rate of said chimeric poxvirus is lower than the neutralization rate of at least two, preferably at least three, more preferably at least four, more preferably at least five, and even more preferably each of the six parental poxvirus strains RPX, CPX, COP, WY, WR and MVA.

For a given tumor and virus, the viral neutralization rate may generally be determined in vitro, 3 to 5 days post-infection of tumor cells with the virus at an MOI of 3×10⁻⁵ to 3. If the anti-poxvirus antibodies are from human serum, said serum may be diluted 10 to 1000 times.

Chimeric Poxvirus with Low Complement-Mediated Virus Neutralization Rate

In another aspect, the invention provides a chimeric poxvirus, wherein the complement-mediated virus neutralization rate of said chimeric poxvirus is lower than the complement-mediated virus neutralization rate measured in the same conditions of at least one of the six parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA), wherein for a given virus and given conditions, the complement-mediated virus neutralization rate (CMV-NT(virus, conditions)) measures the complement induced inhibition of the virus' oncolytic power and is defined as:
CMV-NT(virus, conditions)=virus titer (active serum)/virus titer (heat-inactivated serum).

In a preferred embodiment, the complement-mediated virus neutralization rate of said chimeric poxvirus is lower than the complement-mediated virus neutralization rate measured in the same conditions of the parental COP. More preferably, the complement-mediated virus neutralization rate of said chimeric poxvirus is lower than the complement-mediated virus neutralization rate of at least two, preferably at least three, more preferably at least four, more preferably at least five, and even more preferably each of the six parental poxvirus strains RPX, CPX, COP, WY, WR and MVA.

For a given virus, the complement-mediated virus neutralization rate may be generally determined in vitro, in presence of human serum (active or heat-inactivated) with the virus at a dose of 10⁴ to 10⁸ PFU/mL.

Chimeric Poxvirus with High Syncytia Formation Capacity

In another aspect, the invention provides a chimeric poxvirus, wherein for at least one producer cell (preferably a tumor cell), the syncytia formation capacity of said chimeric poxvirus is higher than the syncytia formation capacity measured in the same conditions and at the same time post-infection of at least one of the six parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA).

In a preferred embodiment, the invention provides a chimeric poxvirus wherein for at least one producer cell (preferably a tumor cell), the syncytia formation capacity of said chimeric poxvirus is higher than the syncytia formation capacity measured in the same conditions and at the same time post-infection of the parental Vaccinia virus strain Copenhagen (COP). More preferably, for at least one producer cell (preferably a tumor cell), the syncytia formation capacity of said chimeric poxvirus is higher than the syncytia formation capacity measured in the same conditions and at the same time post-infection of at least two of the six parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA). For instance, in a preferred embodiment, for at least one producer cell (preferably a tumor cell), the syncytia formation capacity of said chimeric poxvirus is higher than the syncytia formation capacity measured in the same conditions and at the same time post-infection of at least two, preferably at least three, more preferably at least four, at least five, and more preferably each of the six parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA).

For a given tumor and virus, the syncytia formation capacity may generally be determined in vitro, 16 to 96 hours post-infection of tumor cells with the virus at an MOI of 10⁵ to 1.

### Chimeric Poxvirus Combining Several Functional Features of Interest

Chimeric poxviruses according to the invention that combine several of the functional features described above are particularly preferred. The chimeric poxvirus (optionally variant and/or recombinant) of the invention may thus comprise any combination of functional features described herein.

The chimeric poxvirus of the invention may in particular comprise any combination of:
a high oncolytic power as disclosed in the corresponding section above (preferably compared to parental Vaccinia virus strain Copenhagen (COP), to parental Rabbitpox virus strain Utrecht (RPX), to parental COP and RPX, or to all parental poxvirus strains);
a low viral replication in healthy cells (preferably primary cells) as disclosed in the corresponding section above (preferably compared to parental Vaccinia virus strain Copenhagen (COP) or to all parental poxvirus strains);
a high EEV-SC, a high syncytia formation capacity, a high spreading capacity, a low neutralization rate in the presence of poxvirus-specific antibody and/or a low complement-mediated virus neutralization rate as disclosed in the corresponding section above (preferably compared to parental Vaccinia virus strain Copenhagen (COP), to parental Rabbitpox virus strain Utrecht (RPX), to Vaccinia virus strain IHD-J or to all parental poxvirus strains).

In particular, the chimeric poxvirus of the invention may comprise:
a high oncolytic power as disclosed in the corresponding section above and a low viral replication in healthy cells (preferably primary cells) as disclosed in the corresponding section above;
a high oncolytic power as disclosed in the corresponding section above and (a high EEV-SC, a high syncytia formation capacity, a high spreading capacity, a low neutralization rate in the presence of poxvirus-specific antibody and/or a low complement-mediated virus neutralization rate as disclosed in the corresponding section above);
a low viral replication in healthy cells (preferably primary cells) as disclosed in the corresponding section above and (a high EEV-SC, a high syncytia formation capacity, a high spreading capacity, a low neutralization rate in the presence of poxvirus-specific antibody and/or a low complement-mediated virus neutralization rate as disclosed in the corresponding section above); or
a high oncolytic power as disclosed in the corresponding section above, a low viral replication in healthy cells (preferably primary cells) as disclosed in the corresponding section above and (a high EEV-SC, a high syncytia formation capacity, a high spreading capacity, a low neutralization rate in the presence of poxvirus-specific antibody and/or a low complement-mediated virus neutralization rate as disclosed in the corresponding section above).

A particularly preferred chimeric poxvirus according to the invention comprises the following functional features:
for at least one tumor, the oncolytic power of said chimeric poxvirus is higher than the oncolytic power measured in the same conditions and at the same time post-infection of the parental Vaccinia virus strain Copenhagen (COP) or the parental Rabbitpox virus strain Utrecht (RPX);
for at least one healthy cell (preferably primary cell), the viral replication in the healthy cell (preferably primary cell) of said chimeric poxvirus is lower than that of the parental Vaccinia virus strain Copenhagen (COP);
for at least one producer cell (preferably a tumor cell), the EEV-SC of said chimeric poxvirus is higher than the EEV-SC measured in the same conditions and at the same time post-infection of the parental Rabbitpox virus strain Utrecht (RPX) or the Vaccinia virus strain IHD-J;
optionally, for at least one poxvirus-specific antibody and tumor, the neutralization rate of said chimeric poxvirus is lower than the neutralization rate measured in the same conditions and at the same time post-infection of the parental Vaccinia virus strain Copenhagen (COP);
optionally, the complement-mediated virus neutralization rate of said chimeric poxvirus is lower than the complement-mediated virus neutralization rate measured in the same conditions of the parental Vaccinia virus strain Copenhagen (COP); and
optionally, for at least one producer cell (preferably a tumor cell), the syncytia formation capacity of said chimeric poxvirus is higher than the syncytia formation capacity measured in the same conditions and at the same time post-infection of the parental Vaccinia virus strain Copenhagen (COP).

A further particularly preferred chimeric poxvirus according to the invention comprises the following functional features:
for at least one tumor, the oncolytic power of said chimeric poxvirus is higher than the oncolytic power measured in the same conditions and at the same time post-infection of the parental Vaccinia virus strain Copenhagen (COP) or the parental Rabbitpox virus strain Utrecht (RPX);
for at least one healthy cell (preferably primary cell), the viral replication in the healthy cell (preferably primary cell) of said chimeric poxvirus is lower than that of the parental Vaccinia virus strain Copenhagen (COP);
for at least one producer cell (preferably a tumor cell), the syncytia formation capacity of said chimeric poxvirus is higher than the syncytia formation capacity measured in the same conditions and at the same time post-infection of the parental Vaccinia virus strain Copenhagen (COP);
optionally, for at least one producer cell (preferably a tumor cell), the EEV-SC of said chimeric poxvirus is higher than the EEV-SC measured in the same conditions and at the same time post-infection of the parental Rabbitpox virus strain Utrecht (RPX) or the Vaccinia virus strain IHD-J;
optionally, for at least one poxvirus-specific antibody and tumor, the neutralization rate of said chimeric poxvirus is lower than the neutralization rate measured in the same conditions and at the same time post-infection of the parental Vaccinia virus strain Copenhagen (COP); and
optionally, the complement-mediated virus neutralization rate of said chimeric poxvirus is lower than the complement-mediated virus neutralization rate measured in the same conditions of the parental Vaccinia virus strain Copenhagen (COP).

A more particularly preferred chimeric poxvirus according to the invention comprises the following functional features:
for at least one tumor, the oncolytic power of said chimeric poxvirus is higher than the oncolytic power measured in the same conditions and at the same time post-infection of the parental Vaccinia virus strain Copenhagen (COP) or the parental Rabbitpox virus strain Utrecht (RPX);
for at least one healthy cell (preferably primary cell), the viral replication in the healthy cell (preferably primary cell) of said chimeric poxvirus is lower than that of the parental Vaccinia virus strain Copenhagen (COP);
for at least one producer cell (preferably a tumor cell), the EEV-SC of said chimeric poxvirus is higher than the EEV-SC measured in the same conditions and at the same time post-infection of the parental Rabbitpox virus strain Utrecht (RPX) or the Vaccinia virus strain IHD-J;
for at least one producer cell (preferably a tumor cell), the syncytia formation capacity of said chimeric poxvirus is higher than the syncytia formation capacity measured in the same conditions and at the same time post-infection of the parental Vaccinia virus strain Copenhagen (COP);
optionally, for at least one poxvirus-specific antibody and tumor, the neutralization rate of said chimeric poxvirus is lower than the neutralization rate measured in the same conditions and at the same time post-infection of the parental Vaccinia virus strain Copenhagen (COP); and
optionally, the complement-mediated virus neutralization rate of said chimeric poxvirus is lower than the complement-mediated virus neutralization rate measured in the same conditions of the parental Vaccinia virus strain Copenhagen (COP).

The chimeric poxvirus of the invention may alternatively comprise a high therapeutic index as disclosed in the corresponding section above and (a high EEV-SC, a high syncytia formation capacity, a high spreading capacity, a low neutralization rate in the presence of poxvirus-specific antibody and/or a low complement-mediated virus neutralization rate as disclosed in the corresponding section above). A particularly preferred chimeric poxvirus according to the invention comprises the following functional features:
for at least one organ, the therapeutic index of said chimeric poxvirus is higher than the therapeutic index measured in the same conditions and at the same time post-infection of the parental Vaccinia virus strain Copenhagen (COP);
for at least one producer cell (preferably a tumor cell), the EEV-SC of said chimeric poxvirus is higher than the EEV-SC measured in the same conditions and at the same time post-infection of the parental Rabbitpox virus strain Utrecht (RPX) or the Vaccinia virus strain IHD-J;
optionally, for at least one poxvirus-specific antibody and tumor, the neutralization rate of said chimeric poxvirus is lower than the neutralization rate measured in the same conditions and at the same time post-infection of the parental Vaccinia virus strain Copenhagen (COP);
optionally, the complement-mediated virus neutralization rate of said chimeric poxvirus is lower than the complement-mediated virus neutralization rate measured in the same conditions of the parental Vaccinia virus strain Copenhagen (COP); and
optionally, for at least one producer cell (preferably a tumor cell), the syncytia formation capacity of said chimeric poxvirus is higher than the syncytia formation capacity measured in the same conditions and at the same time post-infection of the parental Vaccinia virus strain Copenhagen (COP).

A further particularly preferred chimeric poxvirus according to the invention comprises the following functional features:
for at least one organ, the therapeutic index of said chimeric poxvirus is higher than the therapeutic index measured in the same conditions and at the same time post-infection of the parental Vaccinia virus strain Copenhagen (COP);
for at least one producer cell (preferably a tumor cell), the syncytia formation capacity of said chimeric poxvirus is higher than the syncytia formation capacity measured in the same conditions and at the same time post-infection of the parental Vaccinia virus strain Copenhagen (COP);
optionally, for at least one producer cell (preferably a tumor cell), the EEV-SC of said chimeric poxvirus is higher than the EEV-SC measured in the same conditions and at the same time post-infection of the parental Rabbitpox virus strain Utrecht (RPX) or the Vaccinia virus strain IHD-J;
optionally, for at least one poxvirus-specific antibody and tumor, the neutralization rate of said chimeric poxvirus is lower than the neutralization rate measured in the same conditions and at the same time post-infection of the parental Vaccinia virus strain Copenhagen (COP); and
optionally, the complement-mediated virus neutralization rate of said chimeric poxvirus is lower than the complement-mediated virus neutralization rate measured in the same conditions of the parental Vaccinia virus strain Copenhagen (COP).

A more particularly preferred chimeric poxvirus according to the invention comprises the following functional features:
for at least one organ, the therapeutic index of said chimeric poxvirus is higher than the therapeutic index measured in the same conditions and at the same time post-infection of the parental Vaccinia virus strain Copenhagen (COP);
for at least one producer cell (preferably a tumor cell), the EEV-SC of said chimeric poxvirus is higher than the EEV-SC measured in the same conditions and at the same time post-infection of the parental Rabbitpox virus strain Utrecht (RPX) or the Vaccinia virus strain IHD-J;
for at least one producer cell (preferably a tumor cell), the syncytia formation capacity of said chimeric poxvirus is higher than the syncytia formation capacity measured in the same conditions and at the same time post-infection of the parental Vaccinia virus strain Copenhagen (COP);
optionally, for at least one poxvirus-specific antibody and tumor, the neutralization rate of said chimeric poxvirus is lower than the neutralization rate measured in the same conditions and at the same time post-infection of the parental Vaccinia virus strain Copenhagen (COP); and
optionally, the complement-mediated virus neutralization rate of said chimeric poxvirus is lower than the complement-mediated virus neutralization rate measured in the same conditions of the parental Vaccinia virus strain Copenhagen (COP).
   Chimeric Poxvirus Obtained or Obtainable by the Method of Directed Evolution for Selecting a Chimeric Poxvirus with High Oncolytic Power

In another aspect, the invention provides a chimeric poxvirus, wherein said chimeric poxvirus has been or may be obtained by any embodiment of the method of directed evolution described below.

Chimeric Poxvirus Combining Nucleic Acid, Functional, and/or Method Features

As explained above, the chimeric poxvirus of the invention may comprise any combination of nucleic acid, functional and/or method features described herein.

In particular, a preferred chimeric poxvirus of the invention may comprise:
a nucleic acid sequence having a sequence identity of at least 96,6% (or any other identity percentage disclosed above in the corresponding section) with SEQ ID NO:1; and
one of the following functional features or combinations of functional features:
   a) a high oncolytic power as disclosed in the corresponding section above (preferably compared to parental Vaccinia virus strain Copenhagen (COP), to parental Rabbitpox virus strain Utrecht (RPX) or to all parental poxvirus strains);
   b) a low viral replication in healthy cells (preferably primary cells) as disclosed in the corresponding section above (preferably compared to parental Vaccinia virus strain Copenhagen (COP) or to all parental poxvirus strains);
   c) a high EEV-SC, a high syncytia formation capacity, a high spreading capacity, a low neutralization rate in the presence of poxvirus-specific antibody and/or a low complement-mediated virus neutralization rate as disclosed in the corresponding section above (preferably compared to parental Vaccinia virus strain Copenhagen (COP), to parental Rabbitpox virus strain Utrecht (RPX), to Vaccinia virus strain IHD-J or to all parental poxvirus strains);
   d) a) and b);
   e) a) and c);
   f) b) and c); and
   g) a), b) and c).

In particular, a chimeric poxvirus of the invention may comprise:
a nucleic acid sequence having a sequence identity of at least 96,6% (or any other identity percentage disclosed above in the corresponding section) with SEQ ID NO:1; and
one of the following functional features or combinations of functional features:
   a) for at least one tumor, the oncolytic power of said chimeric poxvirus is higher than the oncolytic power measured in the same conditions and at the same time post-infection of the parental Vaccinia virus strain Copenhagen (COP) or the parental Rabbitpox virus strain Utrecht (RPX);
   b) for at least one healthy cell (preferably primary cell), the viral replication in the healthy cell (preferably primary cell) of said chimeric poxvirus is lower than that of the parental Vaccinia virus strain Copenhagen (COP);
   c) for at least one producer cell (preferably a tumor cell), the EEV-SC of said chimeric poxvirus is higher than the EEV-SC measured in the same conditions and at the same time post-infection of the parental Rabbitpox virus strain Utrecht (RPX) or the Vaccinia virus strain IHD-J;
   d) for at least one poxvirus-specific antibody and tumor, the neutralization rate of said chimeric poxvirus is lower than the neutralization rate measured in the same conditions and at the same time post-infection of the parental Vaccinia virus strain Copenhagen (COP);
   e) the complement-mediated virus neutralization rate of said chimeric poxvirus is lower than the complement-mediated virus neutralization rate measured in the same conditions of the parental Vaccinia virus strain Copenhagen (COP);
   f) for at least one producer cell (preferably a tumor cell), the syncytia formation capacity of said chimeric poxvirus is higher than the syncytia formation capacity measured in the same conditions and at the same time post-infection of the parental Vaccinia virus strain Copenhagen (COP).
   g) a) and b);
   h) a) and c);
   i) a) and d);
   j) a) and e);
   k) a) and f);
   I) b) and c);
   m) b) and d);
   n) b) and e);
   o) b) and f);
   p) c) and d);
   q) c) and e);
   r) c) and f);
   s) a), b) and c);
   t) a), b) and d);
   u) a), b) and e);
   v) a), b) and f);
   w) a), c) and d);
   x) a), c) and f);
   y) b), c) and d);
   z) b), c) and f);
   aa) a), b), c) and d);
   bb) a), b), c) and e);
   cc) a), b), c) and f); and
   dd) any combination thereof.

For all the above embodiments combining a sequence identity of at least 96,6% (or any other identity percentage disclosed above in the corresponding section) with SEQ ID NO:1 and one or more functional features, the poxvirus of the invention may further comprises nucleic acid sequences derived from at least two, at least 3, at least 4, at least 5 or all 6 parental poxvirus strains Rabbitpox virus strain Utrecht (RPX), Cowpox virus strain Brighton (CPX), Vaccinia virus strain Copenhagen (COP), Vaccinia virus strain Western Reserve (WR), Vaccinia virus strain Wyeth (WY) and Modified Vaccinia virus strain Ankara (MVA), preferably one or more of the specific fragments of the parental poxvirus strains defined in the section above relating to the chimeric poxvirus defined by nucleic acid features. For all the above embodiments combining nucleic acid features and one or more functional features, the poxvirus of the invention may further have been obtained by one of the methods of directed evolution disclosed in the next section.

### Chimeric Poxvirus of Strain POXSTG19503 Having Accession Number CNCM-I-5913 Comprising Functional Features

According to a particular embodiment, a chimeric poxvirus of the invention is strain POXSTG19503 having the accession number CNCM-I-5913 and may comprise one of the following functional features or combinations of functional features:
a) a high oncolytic power as disclosed in the corresponding section above (preferably compared to parental Vaccinia virus strain Copenhagen (COP), to parental Rabbitpox virus strain Utrecht (RPX) or to all parental poxvirus strains);
b) a low viral replication in healthy cells (preferably primary cells) as disclosed in the corresponding section above (preferably compared to parental Vaccinia virus strain Copenhagen (COP) or to all parental poxvirus strains);
c) a high EEV-SC, a high syncytia formation capacity, a high spreading capacity, a low neutralization rate in the presence of poxvirus-specific antibody and/or a low complement-mediated virus neutralization rate as disclosed in the corresponding section above (preferably compared to parental Vaccinia virus strain Copenhagen (COP), to parental Rabbitpox virus strain Utrecht (RPX), to Vaccinia virus strain IHD-J or to all parental poxvirus strains);
d) a) and b);
e) a) and c);
f) b) and c); and
g) a), b) and c).

In particular, a preferred chimeric poxvirus of the invention is strain POXSTG19503 having the accession number CNCM-I-5913 and may comprise one of the following functional features or combinations of functional features:
a) for at least one tumor, the oncolytic power of said chimeric poxvirus is higher than the oncolytic power measured in the same conditions and at the same time post-infection of the parental Vaccinia virus strain Copenhagen (COP) or the parental Rabbitpox virus strain Utrecht (RPX);
b) for at least one healthy cell (preferably primary cell), the viral replication in the healthy cell (preferably primary cell) of said chimeric poxvirus is lower than that of the parental Vaccinia virus strain Copenhagen (COP);
c) for at least one producer cell (preferably a tumor cell), the EEV-SC of said chimeric poxvirus is higher than the EEV-SC measured in the same conditions and at the same time post-infection of the parental Rabbitpox virus strain Utrecht (RPX) or the Vaccinia virus strain IHD-J;
d) for at least one poxvirus-specific antibody and tumor, the neutralization rate of said chimeric poxvirus is lower than the neutralization rate measured in the same conditions and at the same time post-infection of the parental Vaccinia virus strain Copenhagen (COP);
e) the complement-mediated virus neutralization rate of said chimeric poxvirus is lower than the complement-mediated virus neutralization rate measured in the same conditions of the parental Vaccinia virus strain Copenhagen (COP);
f) for at least one producer cell (preferably a tumor cell), the syncytia formation capacity of said chimeric poxvirus is higher than the syncytia formation capacity measured in the same conditions and at the same time post-infection of the parental Vaccinia virus strain Copenhagen (COP).
g) a) and b);
h) a) and c);
i) a) and d);
j) a) and e);
k) a) and f);
l) b) and c);
m) b) and d);
n) b) and e);
o) b) and f);
p) c) and d);
q) c) and e);
r) c) and f);
s) a), b) and c);
t) a), b) and d);
u) a), b) and e);
v) a), b) and f);
w) a), c) and d);
x) a), c) and f);
y) b), c) and d);
z) b), c) and f);
aa) a), b), c) and d);
bb) a), b), c) and e);
cc) a), b), c) and f); and
dd) any combination thereof.

For all the above embodiments combining a chimeric poxvirus of strain POXSTG19703 having accession number CNCM-I-5913 and one or more functional features, the poxvirus of the invention may further comprises nucleic acid sequences derived from at least two, at least 3, at least 4, at least 5 or all 6 parental poxvirus strains Rabbitpox virus strain Utrecht (RPX), Cowpox virus strain Brighton (CPX), Vaccinia virus strain Copenhagen (COP), Vaccinia virus strain Western Reserve (WR), Vaccinia virus strain Wyeth (WY) and Modified Vaccinia virus strain Ankara (MVA), preferably one or more of the specific fragments of the parental poxvirus strains defined in the section above relating to the chimeric poxvirus defined by the strain POXSTG19503 having accession number CNCM-I-5913. For all the above embodiments combining a chimeric poxvirus of strain POXSTG19503 having accession number CNCM-I-5913 and one or more functional features, the poxvirus of the invention may further have been obtained by one of the methods of directed evolution method disclosed in the following section.

Method of Directed Evolution for Selecting a Chimeric Poxvirus with High Oncolytic Power

In another aspect, the invention provides a method of directed evolution for obtaining a chimeric poxvirus with high oncolytic power, said method comprising:
(i) infecting a first tumor cell line with a plurality of parental poxvirus strains, wherein said tumor cell line is permissive for each parental poxvirus strain, so as to obtain a first infected tumor cell line;
(ii) amplifying said parental poxvirus strains on said first infected tumor cell line of step (i) during at least 12h (preferably at least 24h) and at most 3 days, so as to obtain one or more distinct chimeric poxviruses through homologous genomic recombination between at least two of said parental poxvirus strains in supernatant;
(iii) collecting the supernatant at the end of step (ii) containing the one or more distinct chimeric poxvirus(es);
(iv) infecting a second tumor cell line with the one or more distinct chimeric poxvirus(es) of the supernatant of step (iii), wherein said second tumor cell line is permissive for each parental poxvirus strain of steps (i) and (ii), so as to obtain a second infected tumor cell line;
(vₐ) amplifying the one or more distinct chimeric poxvirus(es) of step (iv) on said second infected tumor cell line of step (iv) during preferably at least 12 h and at most 24 h and then collecting the supernatant;
(vi) selecting one or more distinct chimeric poxvirus(es) of step (vₐ) having, for at least one third tumor cell line, an oncolytic power higher than the oncolytic power measured in the same conditions and at the same time post-infection of at least one, preferably several, and more preferably all the parental oncolytic poxvirus strains in the first tumor cell line of step (i) and/or in the second tumor cell line of step (iv), wherein for a given tumor, a given virus, given conditions and a given time post-infection, an oncolytic power OP(tumor, virus, conditions, time post-infection) is defined as:
OP(tumor, virus, conditions, time post-infection)=(100-percentage of surviving tumor cells after infection by virus).

More particularly, said method of directed evolution comprises the following steps:
(i) infecting a first tumor cell line with a plurality of parental poxvirus strains, wherein said tumor cell line is permissive for each parental poxvirus strain, so as to obtain a first infected tumor cell line;
(ii) amplifying said parental poxvirus strains on said first infected tumor cell line of step (i) during at least 12h (preferably at least 24h) and at most 3 days, so as to obtain one or more distinct chimeric poxviruses through homologous genomic recombination between at least two of said parental poxvirus strains in supernatant;
(iii′) collecting the supernatant at the end of step (ii) containing the one or more distinct chimeric poxvirus(es), and performing a 5 to 20-fold dilution series so as to obtain at least two diluted supernatants each containing one or more chimeric poxvirus(es);
(iv′) infecting at least two samples of a second tumor cell line with the one or more distinct chimeric poxvirus(es) of each of the diluted supernatants of step (iii′), wherein said second tumor cell line is permissive for each parental poxvirus strain of steps (i) and (ii), so as to obtain at least two samples of the second infected tumor cell line;
(v′ₐ) amplifying the one or more distinct chimeric poxvirus(es) of each of the at least two samples of the second infected tumor cell line of step (iv′) on the second infected tumor cell line of step (iv′) during preferably at least 12 h and at most 24h;
(v′_{b}) collecting the supernatant from the sample of infected second tumor cell line infected with the less diluted supernatant that shows no sign of cytopathic effect, and performing a 5 to 20-fold dilution series;
(v′_{c}) repeating steps (iv), (v′ₐ) and (v′_{b}) until one or more distinct chimeric poxvirus(es) meeting the selection criteria of step (vi) is obtained; and
(vi) selecting one or more distinct chimeric poxvirus(es) of step (v′_{c}) having, for at least one third tumor cell line, an oncolytic power higher than the oncolytic power measured in the same conditions and at the same time post-infection of at least one, preferably several, and more preferably higher than all the parental oncolytic poxvirus strains in the first tumor cell line of step (i) or in the second tumor cell line of step (iv′), wherein for a given tumor, a given virus, given conditions and a given time post-infection an oncolytic power OP(tumor, virus, conditions, time post-infection) is defined as:
OP(tumor, virus, conditions, time post-infection)=(100-percentage of surviving tumor cells after infection by virus).

Even more particularly, said method of directed evolution comprises the following steps:
i) infecting a first tumor cell line with a plurality of parental poxvirus strains, wherein said tumor cell line is permissive for each parental poxvirus strain, so as to obtain a first infected tumor cell line;
(ii) amplifying said parental poxvirus strains on said first infected tumor cell line of step (i) during at least 12h (preferably at least 24h) and at most 3 days, so as to obtain one or more distinct chimeric poxviruses through homologous genomic recombination between at least two of said parental poxvirus strains;
(iii″ₐ) collecting both cells and supernatant at the end of step (ii) containing one or more distinct chimeric poxvirus(es);
(iii″_{b}) infecting a second tumor cell line with both cells and supernatant containing one or more distinct chimeric poxvirus(es) of step (iii″ₐ), wherein said second tumor cell line is permissive for each parental poxvirus strain of steps (i) and (ii), so as to obtain a second infected tumor cell line;
(iii″_{c}) amplifying one or more distinct chimeric poxvirus(es) of step (iii″_{b}) on said second infected tumor cell line of step (iii″_{b}) during at least 48h (preferably at least 72h) and at most 3 days;
(iii″_{d}) collecting a fraction of the cells and supernatant containing one or more distinct chimeric poxvirus(es) of step (iii″_{c});
(iii″ₑ) repeating steps (iii″_{b}), (iii″_{c}) and (iii″d) at least one time;
(iii″′f) collecting the supernatant at the end of step (iii″ₑ) containing one or more distinct chimeric poxvirus(es), and performing a 5 to 20-fold dilution series so as to obtain at least two diluted supernatants;
(iv″) infecting at least two samples of a third tumor cell line with the one or more distinct chimeric poxvirus(es) of each of the diluted supernatants of step (iii″), wherein said third tumor cell line is permissive for each parental poxvirus strain of steps (i) and (ii) so as to obtain at least two samples of the third infected tumor cell line;
(v″ₐ) amplifying the one or more distinct chimeric poxvirus(es) of each of the at least two samples of the third infected tumor cell line of step (iv″) on a third infected tumor cell line of step (iv″) during at least 12 h and at most 24h;
(v″_{b}) collecting the supernatant from the sample of third infected tumor cell line infected with the less diluted supernatant that shows no sign of cytopathic effect, and performing a 5 to 20-fold dilution series;
(v″_{c}) repeating steps (iv″), (v″ₐ) and (v″_{b}) until one or more distinct chimeric poxvirus(es) meeting the selection criteria of step (vi) is obtained; and
(vi) selecting one or more distinct chimeric poxvirus(es) of step (v″_{c}) having, for at least one fourth tumor cell line, an oncolytic power higher than the oncolytic power measured in the same conditions and at the same time post-infection of at least one, preferably several, and more preferably all the parental oncolytic poxvirus strains in the tumor cell line of steps (iv″), wherein for a given tumor, a given virus, given conditions and a given time post-infection, an oncolytic power OP(tumor, virus, conditions, time post-infection) is defined as:
OP(tumor, virus, conditions, time post-infection)=(100−percentage of surviving tumor cells after infection by virus).

In said method of directed evolution, the different parental poxvirus strains are pooled, and the blend is used to infect a first permissive tumor cell line. Several passages are then made in a second permissive tumor cell line. In this general manner, the method facilitates recombination between the different poxviral strains and the generation of new chimeric poxviruses. A selection is then effectuated between the new generated chimeric poxviruses according to their oncolytic power, and optionally other functional features.

Preferably, in the above method of directed evolution, at least two, preferably at least three, preferably at least four, preferably at least five, preferably at least six, such as 2 to 50, 3 to 40, 4 to 30, 5 to 25, 6 to 20, 6 to 19, 6 to 18, 6 to 17, or 6 to 16 different parental poxvirus strains are used for infecting a permissive tumor cell of step (i).

When no more than 16 distinct poxviruses are used as parental poxvirus strains in first step (i), they are preferably selected in the group consisting of Rabbitpox virus strain Utrecht (RPX), Cowpox virus strain Brighton (CPX), Vaccinia virus strain Copenhagen (COP), Vaccinia virus strain Western Reserve (WR), Vaccinia virus strain Wyeth (WY), Modified Vaccinia virus strain Ankara (MVA), Raccoonpox virus strain Herman (RCN), ORF virus strain NZ2 (ORF), Pseudocowpox strain TJS (PCP), Bovine Papular stomatitis virus strain Illinois 721 (BPS), Myxoma virus strain Lausanne (MYX), Squirrelpox virus strain Kilham (SQF), Fowlpox virus strain FP9 (FPV), Swinepox virus strain Kasza (SPV), Yaba-like disease virus strain Davis (YLD), and Cotia virus strain SP An 232 (CTV). Preferably, said parental poxvirus strains are used at a dose comprised between 1.5×10³ PFU (plaque-forming unit) and 1.5×10⁶ PFU, more preferably between 1×10⁴ PFU and 1×10⁶ PFU, and even more preferably between 1.5×10⁴ PFU and 1.5×10⁻⁵ PFU.

In a preferred embodiment, said parental poxvirus strains in first step (i) are orthopoxvirus strains, preferably selected (when no more than 6 distinct orthopoxviruses are used as parental poxvirus strains) in the group consisting of Rabbitpox virus strain Utrecht (RPX), Cowpox virus strain Brighton (CPX), Vaccinia virus strain Copenhagen (COP), Vaccinia virus strain Western Reserve (WR), Vaccinia virus strain Wyeth (WY) and Modified Vaccinia virus strain Ankara (MVA). Preferably, said parental orthopoxvirus strains are used at a dose comprised between 1.5×10³ PFU and 1.5×10⁶ PFU, more preferably between 1×10⁴ PFU and 1×10⁶ PFU, and even more preferably between 1.5×10⁴ PFU and 1.5×10⁻⁵ PFU.

Preferably, in the above method of directed evolution, said permissive tumor cell lines used for generating the chimeric poxvirus of the invention are mammalian cells, said cells being permissive to poxvirus infection and replication.

In the above methods, all the tumor cell line(s) are permissive for the replication of the parental poxvirus strains used in step (i). Permissive tumor cell lines used as first, second, third and optionally fourth tumor cell line in the above methods can be the same or different, preferably the same tumor cell line is used in all steps of the method. Examples of permissive tumor cell lines are A549, CAL-33, HepG2, HCT116, Hela, SK-MEL-1, PANC-1, Hs746T, SK-OV-3 and CV-1. In a preferred embodiment, the first, second, third and optionally fourth permissive tumor cell lines used for generating chimeric poxviruses are A549.

Steps (i), (iv), (iv′), (iii″_{b}) and (iv″) comprise the infection of permissive tumor cell lines with several distinct poxvirus strains, and the amplification of said poxvirus strains. In such case, genetic exchange, also called “shuffling”, may happen between various strains, leading to the generation of chimeric poxviruses. The use of a permissive tumor cell line results in a high frequency of genetic exchanges between the different viral strains, including point mutations and recombination events. As a result, the amplification step allows the enrichment of the generated chimeric poxviruses increased oncolytic power in the tumor cell line used for amplification.

In a preferred embodiment, the methods of directed evolution according to the invention include infecting A549 tumor cell line with Rabbitpox virus strain Utrecht (RPX), Cowpox virus strain Brighton (CPX), Vaccinia virus strain Copenhagen (COP), Vaccinia virus strain Western Reserve (WR), Vaccinia virus strain Wyeth (WY), Modified Vaccinia virus strain Ankara (MVA), Raccoonpox virus strain Herman (RCN), ORF virus strain NZ2 (ORF), Pseudocowpox strain TJS (PCP), Bovine Papular stomatitis virus strain Illinois 721 (BPS), Myxoma virus strain Lausanne (MYX), Squirrelpox virus strain Kilham (SQF), Fowlpox virus strain FP9 (FPV), Swinepox virus strain Kasza (SPV), Yaba-like disease virus strain Davis (YLD) and Cotia virus strain SP An 232 (CTV) in step (i)). More preferably, step (i) of said method includes infecting A549 tumor cell line with 1.5×10⁴ PFU of Rabbitpox virus strain Utrecht (RPX), 1.5×10⁴ PFU of Cowpox virus strain Brighton (CPX), 1.5×10⁴ PFU of Vaccinia virus strain Copenhagen (COP), 1.5×10⁴ PFU of Vaccinia virus strain Western Reserve (WR), 1.5×10⁴ PFU of Vaccinia virus strain Wyeth (WY), 1.5×10⁴ PFU of Modified Vaccinia virus strain Ankara (MVA), 1.5×10⁴ PFU of Raccoonpox virus strain Herman (RCN), 1.5×10⁴ PFU of ORF virus strain NZ2 (ORF), 1.5×10⁴ PFU of Pseudocowpox strain TJS (PCP), 1.5×10⁴ PFU of Bovine Papular stomatitis virus strain Illinois 721 (BPS), 1.5×10⁴ PFU of Myxoma virus strain Lausanne (MYX), 1.5×10⁴ PFU of Squirrelpox virus strain Kilham (SQF), 1.5×10⁴ PFU of Fowlpox virus strain FP9 (FPV), 1.5×10⁴ PFU of Swinepox virus strain Kasza (SPV), 1.5×10⁴ PFU of Yaba-like disease virus strain Davis (YLD), and 1.5×10⁴ PFU of Cotia virus strain SP An 232 (CTV). The above embodiments correspond to the method used in the examples for generated chimeric poxvirus POXSTG19503.

However, in the case of POXSTG19503, only orthopoxvirus parental strains recombined with each other. As a result, only orthopoxvirus parental strains may be used. Therefore, in another preferred embodiment, the method of directed evolution includes infecting A549 tumor cell line with Rabbitpox virus strain Utrecht (RPX), Cowpox virus strain Brighton (CPX), Vaccinia virus strain Copenhagen (COP), Vaccinia virus strain Western Reserve (WR), Vaccinia virus strain Wyeth (WY) and Modified Vaccinia virus strain Ankara (MVA) in step (i) and infecting A549 tumor cell lines with one or more distinct chimeric poxvirus(es) in step (iii). More preferably, step (i) of said method includes infecting A549 tumor cell line with 1.5×10⁴ PFU of Rabbitpox virus strain Utrecht (RPX), 1.5×10⁴ PFU of Cowpox virus strain Brighton (CPX), 1.5×10⁴ PFU of Vaccinia virus strain Copenhagen (COP), 1.5×10⁴ PFU of Vaccinia virus strain Western Reserve (WR), 1.5×10⁴ PFU of Vaccinia virus strain Wyeth (WY) and 1.5×10⁴ PFU of Modified Vaccinia virus strain Ankara (MVA).

Steps (iii), (vₐ), (iii′), (v′_{b}), (v′_{c}), (iii″ₐ), (ii″ₐ), (iii″), (v″_{b}) and (v″_{c}) comprise the collection of the supernatant comprising one or more oncolytic chimeric poxviruses. In steps (vₐ), (v′_{b}), (v′_{c}), (v″_{b}) and (v″_{c}), the supernatant is collected at 12 to 24 hours post-infection. At this time post-infection, the tumor permissive cell line has generally not yet been lysed by the one or more oncolytic chimeric poxviruses, so that the one or more oncolytic chimeric poxviruses present in the collected supernatant should mainly comprise EEV particles. The specific collection of the supernatant at 12 to 24 hours post-infection in steps (vₐ), (v′_{b}), (v′_{c}), (v″_{b}) and (v″_{c}) thus ensures that not only highly oncolytic chimeric poxviruses are selected, but also that chimeric poxviruses with high EEV-SC (and as a result high spreading capacity and low neutralization rate, as a high EEV-SC is correlated to a high spreading capacity and a low neutralization rate) are selected. Repeating several times (infection by supernatant, amplification during 12 to 24 hours and collection of supernatant), as in step (v′_{c}) or (v″_{c}), thus enriches in highly oncolytic chimeric poxviruses with high EEV-SC (and as a result high spreading capacity and low neutralization rate).

In steps (iii′), (v′_{b}), (v′_{c}), (iii″_{f}), (v″_{b}) and (v″_{c}), a 5 to 20-fold dilution series of the collected supernatant is performed, i.e.: part of the supernatant is diluted 5-20 times, part of this 5-20 diluted supernatant is diluted 5-20 times resulting in a 25-400 diluted supernatant. The number of serial dilutions is preferably comprised between 1 and 5, more preferably between 2 and 4, such as 2, 3 and 4. Preferably, the fold dilution between each serial dilution is comprised between 6 and 18, more preferably between 7 and 15, more preferably between 8 and 12. A 10-fold dilution series may notably be used. Preferably, the dilution factor between the undiluted supernatant and the most diluted supernatant in the dilution series is comprised between 100 and 10000, preferably between 200 and 8000, between 300 and 6000, between 400 and 4000, between 500 and 2000, or between 750 and 1500.

In one embodiment, step (iii″ₑ) is repeated between 1 and 30 times, preferably between 1 and 25 times, more preferably between 1 and 20 times, more preferably between 1 and 15 times, more preferably between 1 and 10 times, more preferably between 1 and 5 times, and even more preferably between 1 and 3 times.

In one embodiment, steps (v′_{c}) and (v″_{c}) are repeated between 1 and 200 times, preferably between 2 and 150 times, more preferably between 3 and 100 times, more preferably between 4 and 50 times, more preferably between 5 and 40 times, more preferably between 6 and 30 times, more preferably between 6 and 20 times, more preferably between 7 and 15 times, more preferably between 8 and 12 times.

In any one of the above methods of directed evolution, the selection of step (vi) of the one or more distinct chimeric poxvirus(es) may comprise obtaining viral clones. In a preferred embodiment, said obtention of viral clones may be the result of dilution, isolation and amplification of the one or more distinct chimeric poxvirus(es) of step (v). Alternatively, or in combination, the selection of step (vi) may comprise testing the oncolytic power of the distinct chimeric poxvirus(es) of step (v) on one or more tumor cell lines.

In one embodiment, the method of directed evolution further comprises the use of at least one mutagenic agent in one or more of step(s) (i), (ii), (iii), (iii′), (iii″ₐ), (iii″_{b}), (iii″_{c}), (iii″ₐ), (iii″ₑ), (iii″_{f}), (iv), (iv), (iv″), (v), (vₐ), (v′_{b}), (vc), (v″ₐ), (v″_{b}), (v″_{c}) and (vi) in order to increase the genetic diversity. For instance, said mutagenic agents may be selected from physical, chemical and biological agents. More preferably, said physical agents are selected in the group consisting of ultraviolet radiations, ionizing radiations and radioactive decays, said chemical agents are selected in the group consisting of urea, nitrosourea, reactive oxygen species, deaminating agents, polycyclic aromatic hydrocarbon, alkylating agents, aromatic amines, alkaloid, bromine, sodium azide and benzene, and said biological agents are selected in the group consisting of DNA base analogues and transposons. Other physic, chemical and biological mutagenic agents known by the skilled person may be used in the context of the invention.

### Variant Chimeric Poxvirus

Various oncolytic poxvirus variants with improved properties (in particular lower replication in healthy cells (preferably primary cells) and thus higher therapeutic index) are known in the art, and variants of the chimeric poxvirus of the invention with similar alterations in one or more viral gene(s) may thus be advantageous for use in the treatment of proliferative diseases, such as cancer.

In another aspect, the present invention thus relates to a variant chimeric poxvirus, i.e. a chimeric poxvirus according to the invention that has been modified by altering one or more poxviral gene(s).

In an embodiment, the modification(s) of the variant chimeric poxvirus of the present invention preferably lead(s) to the synthesis of a defective protein unable to ensure the activity of the protein produced under normal conditions by the unmodified gene (or lack of synthesis). Exemplary modifications are disclosed in the literature with the goal of altering viral genes involved in DNA metabolism, host virulence, IFN pathway (e.g.: Guse et al., 2011, Expert Opinion Biol. Ther.11 (5): 595-608) and the like. Modifications for altering a viral locus encompass deletion, mutation and/or substitution of one or more nucleotide(s) (contiguous or not) within the viral gene or its regulatory elements. Modification(s) can be made by a number of ways known to those skilled in the art using conventional techniques.

In the context of the invention, the variant chimeric poxvirus can be rendered defective for a particular locus by a number of ways including substitution(s), deletion(s) and/or insertions of one or more nucleotide(s) present in this locus. For example, insertion of a polynucleotide in the locus may disrupt the open reading frame (ORF) encoded by the nucleic acid sequence of the locus. Partial or total deletion of a particular locus is also appropriate to generate a variant chimeric poxvirus defective for a particular locus.

The variant chimeric poxvirus may notably be partially or totally defective in one or more specific loci.

### Variant Chimeric Poxvirus Defective in the J2R Locus

Preferably, the variant chimeric poxvirus is defective in the J2R locus, where is located the thymidine kinase (TK) encoding-gene (similar to J2R gene in COP). The term “defective in the J2R locus” means that the chimeric poxvirus of the invention encodes a nonfunctional thymidine kinase enzyme or does not encode any thymidine kinase enzyme. Preferably, the chimeric poxvirus of the invention does not encode any thymidine kinase enzyme. The TK enzyme is involved in the synthesis of deoxyribonucleotides. TK is needed for viral replication in healthy cells (preferably primary cells) as these cells have generally low concentration of nucleotides whereas it is dispensable in dividing cells (e.g.: tumor cells) that contain high nucleotide concentration. The alteration of the expression (e.g.: deletion of the J2R locus) or functionality of the TK enzyme may thus improve the tumor selectivity of the chimeric poxvirus by reduction of said chimeric poxvirus replication in non-tumor cells. In a preferred embodiment, the deletion of the TK encoding gene has no or low impact on the lytic activity of said virus. Consequently, a variant chimeric poxvirus defective in the J2R locus may have a lower replication in healthy cells (preferably primary cells) and, as a result, an improved therapeutic index.

In this case, the variant chimeric poxvirus preferably has at least 99%, at least 99,1%, at least 99,2%, at least 99,3%, at least 99,4%, at least 99,5%, at least 99,6%, at least 99,7%, at least 99,8%, at least 99,9%, at least 99,91%, at least 99,92%, at least 99,93%, at least 99,94%, at least 99,95%, at least 99,96%, at least 99,97%, at least 99,98%, at least 99,99%, or even 100% of identity with SEQ ID NO: 8. SEQ ID NO:8 is the sequence of the core region and one of the ITRs of POXSTG19508, which corresponds to POXSTG19503, except that the GFP::FCU1 sequence has been inserted into the J2R locus.

In a further embodiment, the variant chimeric poxvirus is strain POXSTG19503 having accession number CNCM-I-5913 and is defective in the J2R locus.

Preferably, for at least one tumor, the oncolytic power of a variant chimeric poxvirus defective in the J2R locus according to the invention is higher than the oncolytic power measured in the same conditions and at the same time post-infection of at least one of the variant oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) defective in the J2R locus. Preferably, for at least one tumor, the oncolytic power of said variant chimeric poxvirus defective in the J2R locus is higher than the oncolytic power measured in the same conditions and at the same time post-infection of a variant parental Vaccinia virus strain Copenhagen (COP) defective in the J2R locus or a variant parental Rabbitpox virus strain Utrecht (RPX) defective in the J2R locus. More preferably, for at least one tumor, the oncolytic power of said variant chimeric poxvirus defective in the J2R locus is higher than the oncolytic powers measured in the same conditions and at the same time post-infection of at least two of the variant oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus. For instance, for at least one tumor, the oncolytic power of said variant chimeric poxvirus defective in the J2R locus is higher than the oncolytic powers measured in the same conditions and at the same time post-infection of a variant parental Vaccinia virus strain Copenhagen (COP) defective in the J2R locus and a variant parental Rabbitpox virus strain Utrecht (RPX) defective in the J2R locus. In a more preferred embodiment, for at least one tumor, the oncolytic power of said variant chimeric poxvirus defective in the J2R locus is higher than the oncolytic powers measured in the same conditions and at the same time post-infection of at least three, more preferably at least four, and even more preferably all five of the variant parental oncolytic poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus.

For a given tumor and virus, the oncolytic power may generally be determined in vitro, 3 to 5 days post-infection of tumor cells with the virus at an MOI of 10⁵ to 10⁻².

In a specific aspect of this embodiment, the oncolytic power of said variant chimeric poxvirus defective in the J2R locus is higher than the oncolytic power measured in the same conditions and at the same time post-infection of at least one of the variant oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) defective in the J2R locus in at least one tumor cell line selected from A549, HCT116 and HepG2. Preferably, for at least one tumor selected from A549, HCT116 and HepG2, the oncolytic power of said variant chimeric poxvirus defective in the J2R locus is higher than the oncolytic power measured in the same conditions and at the same time post-infection of a variant parental Vaccinia virus strain Copenhagen (COP) defective in the J2R locus or a variant parental Rabbitpox virus strain Utrecht (RPX) defective in the J2R locus. More preferably, for at least one tumor cell line selected from A549, HCT116 and HepG2, the oncolytic power of said variant chimeric poxvirus defective in the J2R locus is higher than the oncolytic powers measured in the same conditions and at the same time post-infection of at least two of the variant oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus. For instance, for at least one tumor cell line selected from A549, HCT116 and HepG2, the oncolytic power of said variant chimeric poxvirus defective in the J2R locus is higher than the oncolytic powers measured in the same conditions and at the same time post-infection of a variant parental Vaccinia virus strain Copenhagen (COP) defective in the J2R locus and a variant parental Rabbitpox virus strain Utrecht (RPX) defective in the J2R locus. In a more preferred embodiment, for at least one tumor cell line selected from A549, HCT116 and HepG2, the oncolytic power of said variant chimeric poxvirus defective in the J2R locus is higher than the oncolytic powers measured in the same conditions and at the same time post-infection of at least three, more preferably at least four, and even more preferably all five of the variant parental oncolytic poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus.

Preferably, in this embodiment, the oncolytic power is evaluated 4 days post infection and at an MOI of 10⁵ to 10⁴, preferably 10⁻⁵ or 10⁻⁴. More preferably, in this embodiment, the tumor cell lines are cultured at 37° C., 5% CO₂, in Dulbecco's Modified Eagle Medium (DMEM) with 10% of Fetal Calf Serum (FCS).

In a more specific aspect of this embodiment:
a) on A549 and at an MOI of 10⁵, the difference between the oncolytic power of said variant chimeric poxvirus defective in the J2R locus and the oncolytic power measured in the same conditions and at the same time post-infection of at least one of the variant oncolytic parental poxvirus strains RPX, CPX, COP, WY and WR defective in the J2R locus is at least 39%. Preferably, the difference between the oncolytic power of said variant chimeric poxvirus defective in the J2R locus and the oncolytic power measured in the same conditions and at the same time post-infection of a variant parental COP defective in the J2R locus or a variant parental RPX defective in the J2R locus is at least 39%. More preferably, the difference between the oncolytic power of said variant chimeric poxvirus defective in the J2R locus and the oncolytic power measured in the same conditions and at the same time post-infection of at least two of the variant oncolytic parental poxvirus strains RPX, CPX, COP, WY and WR all defective in the J2R locus is at least 39%. For instance, the difference between the oncolytic power of said variant chimeric poxvirus defective in the J2R locus and the oncolytic power measured in the same conditions and at the same time post-infection of a variant parental COP defective in the J2R locus and a variant parental RPX defective in the J2R locus is at least 39%. In a more preferred embodiment, the difference between the oncolytic power of said variant chimeric poxvirus defective in the J2R locus and the oncolytic power measured in the same conditions and at the same time post-infection of at least three, more preferably at least four, and even more preferably all five of the variant parental oncolytic poxvirus strains RPX, CPX, COP, WY and WR all defective in the J2R locus is at least 39%; and/or
b) on A549 and at an MOI of 10⁻⁴, the difference between the oncolytic power of said variant chimeric poxvirus defective in the J2R locus and the oncolytic power measured in the same conditions and at the same time post-infection of at least one of the variant oncolytic parental poxvirus strains RPX, CPX, COP, WY and WR defective in the J2R locus is at least 13%. Preferably, the difference between the oncolytic power of said variant chimeric poxvirus defective in the J2R locus and the oncolytic power measured in the same conditions and at the same time post-infection of a variant parental COP defective in the J2R locus or a variant parental RPX defective in the J2R locus is at least 20%. More preferably, the difference between the oncolytic power of said variant chimeric poxvirus defective in the J2R locus and the oncolytic power measured in the same conditions and at the same time post-infection of at least two of the variant oncolytic parental poxvirus strains RPX, CPX, COP, WY and WR all defective in the J2R locus is at least 13%. For instance, the difference between the oncolytic power of said variant chimeric poxvirus defective in the J2R locus and the oncolytic power measured in the same conditions and at the same time post-infection of a variant parental COP defective in the J2R locus and a variant parental RPX defective in the J2R locus is at least 20%. In a more preferred embodiment, the difference between the oncolytic power of said variant chimeric poxvirus defective in the J2R locus and the oncolytic power measured in the same conditions and at the same time post-infection of at least three, more preferably at least four, and even more preferably all five of the variant parental oncolytic poxvirus strains RPX, CPX, COP, WY and WR all defective in the J2R locus is at least 13%; and/or
c) on HCT116 and at an MOI of 10⁵, the difference between the oncolytic power of said variant chimeric poxvirus defective in the J2R locus and the oncolytic power measured in the same conditions and at the same time post-infection of at least one of the variant oncolytic parental poxvirus strains RPX, CPX, COP, WY and WR defective in the J2R locus is at least 80%. Preferably, the difference between the oncolytic power of said variant chimeric poxvirus defective in the J2R locus and the oncolytic power measured in the same conditions and at the same time post-infection of a variant parental COP defective in the J2R locus or a variant parental RPX defective in the J2R locus is at least 80%. More preferably, the difference between the oncolytic power of said variant chimeric poxvirus defective in the J2R locus and the oncolytic power measured in the same conditions and at the same time post-infection of at least two of the variant oncolytic parental poxvirus strains RPX, CPX, COP, WY and WR all defective in the J2R locus is at least 80%. For instance, the difference between the oncolytic power of said variant chimeric poxvirus defective in the J2R locus and the oncolytic power measured in the same conditions and at the same time post-infection of a variant parental COP defective in the J2R locus and a variant parental RPX defective in the J2R locus is at least 80%. In a more preferred embodiment, the difference between the oncolytic power of said variant chimeric poxvirus defective in the J2R locus and the oncolytic power measured in the same conditions and at the same time post-infection of at least three, more preferably at least four, and even more preferably all five of the variant parental oncolytic poxvirus strains RPX, CPX, COP, WY and WR all defective in the J2R locus is at least 80%; and/or
d) on HCT116 and at an MOI of 10⁴, the difference between the oncolytic power of said variant chimeric poxvirus defective in the J2R locus and the oncolytic power measured in the same conditions and at the same time post-infection of at least one of the variant oncolytic parental poxvirus strains RPX, CPX, COP, WY and WR defective in the J2R locus is at least 33%. Preferably, the difference between the oncolytic power of said variant chimeric poxvirus defective in the J2R locus and the oncolytic power measured in the same conditions and at the same time post-infection of a variant parental COP defective in the J2R locus or a variant parental RPX defective in the J2R locus is at least 33%. More preferably, the difference between the oncolytic power of said variant chimeric poxvirus defective in the J2R locus and the oncolytic power measured in the same conditions and at the same time post-infection of at least two of the variant oncolytic parental poxvirus strains RPX, CPX, COP, WY and WR all defective in the J2R locus is at least 33%. For instance, the difference between the oncolytic power of said variant chimeric poxvirus defective in the J2R locus and the oncolytic power measured in the same conditions and at the same time post-infection of a variant parental COP defective in the J2R locus and a variant parental RPX defective in the J2R locus is at least 33%. In a more preferred embodiment, the difference between the oncolytic power of said variant chimeric poxvirus defective in the J2R locus and the oncolytic power measured in the same conditions and at the same time post-infection of at least three, more preferably at least four, and even more preferably all five of the variant parental oncolytic poxvirus strains RPX, CPX, COP, WY and WR all defective in the J2R locus is at least 33%; and/or
e) on HepG2 and at an MOI of 10⁵, the difference between the oncolytic power of said variant chimeric poxvirus defective in the J2R locus and the oncolytic power measured in the same conditions and at the same time post-infection of at least one of the variant oncolytic parental poxvirus strains RPX, CPX, COP, WY and WR defective in the J2R locus is at least 66%. Preferably, the difference between the oncolytic power of said variant chimeric poxvirus defective in the J2R locus and the oncolytic power measured in the same conditions and at the same time post-infection of a variant parental COP defective in the J2R locus or a variant parental RPX defective in the J2R locus is at least 66%. More preferably, the difference between the oncolytic power of said variant chimeric poxvirus defective in the J2R locus and the oncolytic power measured in the same conditions and at the same time post-infection of at least two of the variant oncolytic parental poxvirus strains RPX, CPX, COP, WY and WR all defective in the J2R locus is at least 66%. For instance, the difference between the oncolytic power of said variant chimeric poxvirus defective in the J2R locus and the oncolytic power measured in the same conditions and at the same time post-infection of a variant parental COP defective in the J2R locus and a variant parental RPX defective in the J2R locus is at least 66%. In a more preferred embodiment, the difference between the oncolytic power of said variant chimeric poxvirus defective in the J2R locus and the oncolytic power measured in the same conditions and at the same time post-infection of at least three, more preferably at least four, and even more preferably all five of the variant parental oncolytic poxvirus strains RPX, CPX, COP, WY and WR all defective in the J2R locus is at least 66%; and/or
f) on HepG2 and at an MOI of 10⁴, the difference between the oncolytic power of said variant chimeric poxvirus defective in the J2R locus and the oncolytic power measured in the same conditions and at the same time post-infection of at least one of the variant oncolytic parental poxvirus strains RPX, CPX, COP, WY and WR defective in the J2R locus is at least 29%. Preferably, the difference between the oncolytic power of said variant chimeric poxvirus defective in the J2R locus and the oncolytic power measured in the same conditions and at the same time post-infection of a variant parental COP defective in the J2R locus or a variant parental RPX defective in the J2R locus is at least 29%. More preferably, the difference between the oncolytic power of said variant chimeric poxvirus defective in the J2R locus and the oncolytic power measured in the same conditions and at the same time post-infection of at least two of the variant oncolytic parental poxvirus strains RPX, CPX, COP,

WY and WR all defective in the J2R locus is at least 29%. For instance, the difference between the oncolytic power of said variant chimeric poxvirus defective in the J2R locus and the oncolytic power measured in the same conditions and at the same time post-infection of a variant parental COP defective in the J2R locus and a variant parental RPX defective in the J2R locus is at least 29%. In a more preferred embodiment, the difference between the oncolytic power of said variant chimeric poxvirus defective in the J2R locus and the oncolytic power measured in the same conditions and at the same time post-infection of at least three, more preferably at least four, and even more preferably all five of the variant parental oncolytic poxvirus strains RPX, CPX, COP, WY and WR all defective in the J2R locus is at least 29%.

Preferably, in this embodiment, the oncolytic power is evaluated 4 days post infection.

Alternatively or in combination, for at least one healthy cell (preferably primary cell), the viral replication in the healthy cell (preferably primary cell) of the variant chimeric poxvirus defective in the J2R locus is lower than that of at least one of the five variant oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus. Preferably, for at least one healthy cell (preferably primary cell), the viral replication in the healthy cell (preferably primary cell) of the variant chimeric poxvirus defective in the J2R locus is lower than that of the variant parental Vaccinia virus strain Copenhagen (COP) defective in the J2R locus. In a preferred embodiment, for at least one healthy cell (preferably primary cell), the viral replication in the healthy cell (preferably primary cell) of the variant chimeric poxvirus defective in the J2R locus is lower than that of at least two, more preferably at least three, more preferably at least four, and even more preferably all five of the variant oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus.

For a given tumor or healthy cells (preferably primary cells), the viral replication may generally be determined in vitro, 2 to 8 days post-infection of tumor or healthy cells (preferably primary cells) with the virus at an MOI of 10⁻⁵ to 10⁻².

In a specific aspect of this embodiment, the viral replication in healthy cells (preferably primary cells) selected from skin cells and hepatocytes of the variant chimeric poxvirus defective in the J2R locus is lower than that of at least one of the five variant oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus. Preferably, for at least one healthy cell (preferably primary cell) selected from skin cells and hepatocytes, the viral replication in said healthy cell (preferably primary cell) selected from skin cells and hepatocytes of the variant chimeric poxvirus defective in the J2R locus is lower than that of the variant parental Vaccinia virus strain Copenhagen (COP) defective in the J2R locus. In a preferred embodiment, for at least one healthy cell (preferably primary cell) selected from skin cells and hepatocytes, the viral replication in said healthy cell (preferably primary cell) selected from skin cells and hepatocytes of the variant chimeric poxvirus defective in the J2R locus is lower than that of at least two, preferably at least three, more preferably at least four, and even more preferably all five of the variant oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus.

In a more specific embodiment of this aspect, the viral replication of said variant chimeric poxvirus defective in the J2R locus is:
a) at least 1.6 times lower in at least in at least one healthy cell line (preferably primary cell line) selected from skin cells and hepatocytes than that of at least one of the five variant oncolytic parental poxvirus strains RPX, CPX, COP, WY and WR all defective in the J2R locus, preferably than that of the variant oncolytic parental COP defective in the J2R locus, more preferably than that of at least two, more preferably at least three, more preferably at least four, and even more preferably each of the five variant oncolytic parental poxvirus strains RPX, CPX, COP, WY and WR all defective in the J2R locus; and/or
b) at least 4.5 times lower in hepatocytes than that of at least one of the five variant oncolytic parental poxvirus strains RPX, CPX, COP, WY and WR all defective in the J2R locus, preferably than that of the variant oncolytic parental COP defective in the J2R locus, more preferably than that of at least two, more preferably at least three, more preferably at least four, and even more preferably each of the five variant oncolytic parental poxvirus strains RPX, CPX, COP, WY and WR all defective in the J2R locus.

Preferably, in this embodiment, the viral replication is evaluated in vitro 3 days post infection and at a MOI of 10⁻⁴ on HepG2, or 7 days post infection and at 10⁵ PFU on human skin model. More preferably, in this embodiment, the tumor cell lines are cultured at 37° C., 5% CO₂, in Dulbecco's Modified Eagle Medium (DMEM) with 10% of Fetal Calf Serum (FCS).

Alternatively or in combination, for at least one organ, the therapeutic index of said variant chimeric poxvirus defective in the J2R locus is higher than the therapeutic index measured in the same conditions and at the same time post-infection of at least one of the variant parental oncolytic poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus. Preferably, for at least one organ, the therapeutic index of said variant chimeric poxvirus defective in the J2R locus is higher than the therapeutic index measured in the same conditions and at the same time post-infection of the variant parental Vaccinia virus strain Copenhagen (COP) defective in the J2R locus. In a more preferred embodiment, for at least one organ, the therapeutic index of said variant chimeric poxvirus defective in the J2R locus is higher than the therapeutic index measured in the same conditions and at the same time post-infection of at least two, more preferably at least three, more preferably at least four, and even more preferably all five of the variant parental oncolytic poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus.

For a given organ and virus, the therapeutic index may generally be determined in vitro, 2 to 8 days post-infection of tumor or healthy cells (preferably primary cells) with the virus at an MOI of 10⁵ to 10⁻².

In a specific aspect of this embodiment, the hepatic therapeutic index of said variant chimeric poxvirus defective in the J2R locus is higher than the hepatic therapeutic index measured in the same conditions and at the same time post-infection of at least one of the variant parental oncolytic poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus. Preferably, the hepatic therapeutic index of said variant chimeric poxvirus defective in the J2R locus is higher than the hepatic therapeutic index measured in the same conditions and at the same time post-infection of the variant parental Vaccinia virus strain Copenhagen (COP) defective in the J2R locus. In a more preferred embodiment, the hepatic therapeutic index of said variant chimeric poxvirus defective in the J2R locus is higher than the hepatic therapeutic index measured in the same conditions and at the same time post-infection of at least two, more preferably at least three, more preferably at least four, and even more preferably all five of the variant parental oncolytic poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus.

Preferably, the hepatic therapeutic index of said variant chimeric poxvirus defective in the J2R locus is at least 9 times higher than the hepatic therapeutic index measured in the same conditions and at the same time post-infection of at least one of the variant parental oncolytic poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus. Preferably, the hepatic therapeutic index of said variant chimeric poxvirus defective in the J2R locus is at least 9 times higher than the hepatic therapeutic index measured in the same conditions and at the same time post-infection of the variant parental Vaccinia virus strain Copenhagen (COP) defective in the J2R locus. In a more preferred embodiment, the hepatic therapeutic index of said variant chimeric poxvirus defective in the J2R locus is at least 9 times higher than the hepatic therapeutic index measured in the same conditions and at the same time post-infection of at least two, preferably at least three, more preferably at least four, and even more preferably all five of the variant parental oncolytic poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus.

More preferably, in this embodiment, the hepatic therapeutic index is measured in vitro in a setting in which the variant chimeric poxvirus is added respectively to the HepG2 tumor cells at a MOI of 10⁻⁵ and to the healthy hepatocytes (preferably primary hepatocytes) at an MOI of 10⁴ and the replication of the variant chimeric poxvirus in the HepG2 tumor cells and in the healthy hepatocytes (preferably primary hepatocytes) is measured 3 days post infection. Even more preferably, in this embodiment, the HepG2 tumor cell lines are cultured at 37° C., 5% CO₂, in Dulbecco's Modified Eagle Medium (DMEM) with 10% of Fetal Calf Serum (FCS), and the healthy hepatocytes (preferably primary hepatocytes) are cultured at 37° C., 5% CO₂, in basal hepatic cell medium (BIOPREDICS catalogue reference MIL600) and additives for hepatocytes culture medium (BIOPREDICS catalogue reference ADD222C). Optionally, the variant chimeric poxvirus defective in the J2R locus has a higher hepatic therapeutic index than the corresponding hepatic therapeutic indexes of the wild-type chimeric poxvirus not defective in the J2R locus.

Alternatively or in combination, for at least one producer cell (preferably a tumor cell), the EEV-SC of a variant chimeric poxvirus defective in the J2R locus is higher than the EEV-SC measured in the same conditions and at the same time post-infection of at least one of the variant parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA) all defective in the J2R locus. Preferably, for at least one producer cell (preferably a tumor cell), the EEV-SC of a variant chimeric poxvirus defective in the J2R locus is higher than the EEV-SC measured in the same conditions and at the same time post-infection of at least one of the variant parental COP defective in the J2R locus or the variant parental Rabbitpox virus strain Utrecht (RPX) defective in the J2R locus. In a preferred embodiment, for at least one producer cell (preferably a tumor cell), the EEV-SC of a variant chimeric poxvirus defective in the J2R locus is higher than the EEV-SC measured in the same conditions and at the same time post-infection of at least two of the variant parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA) all defective in the J2R locus. For instance, for at least one producer cell (preferably a tumor cell), the EEV-SC of a variant chimeric poxvirus defective in the J2R locus is higher than the EEV-SC measured in the same conditions and at the same time post-infection of at least one of the variant parental Vaccinia virus strain Copenhagen (COP) defective in the J2R locus and of the variant parental Rabbitpox virus strain Utrecht (RPX) defective in the J2R locus. In a more preferred embodiment, for at least one producer cell (preferably a tumor cell), the EEV-SC of a variant chimeric poxvirus defective in the J2R locus is higher than the EEV-SC measured in the same conditions and at the same time post-infection of at least three, preferably at least four, more preferably at least five and even more preferably all six of the variant parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia virus strain Ankara (MVA) all defective in the J2R locus.

For a given tumor and virus, the EEV-SC may generally be determined in vitro, 16 to 24 hours post-infection of tumor cells with the virus at an MOI of 10⁻⁴ to 10⁻¹.

In a specific aspect of this embodiment, in A549 tumor cell line, the EEV-SC of a variant chimeric poxvirus defective in the J2R locus is higher than the EEV-SC measured in the same conditions and at the same time post-infection of at least one of the variant parental poxvirus strains RPX, CPX, COP, WY, WR and MVA, all defective in the J2R locus. Preferably, in A549 tumor cell line, the EEV-SC of a variant chimeric poxvirus defective in the J2R locus is higher than the EEV-SC measured in the same conditions and at the same time post-infection of at least one of the variant parental COP defective in the J2R locus or the variant parental RPX defective in the J2R locus. In a preferred embodiment, in A549 tumor cell line, the EEV-SC of a variant chimeric poxvirus defective in the J2R locus is higher than the EEV-SC measured in the same conditions and at the same time post-infection of at least two of the variant parental poxvirus strains RPX, CPX, COP, WY, WR and MVA all defective in the J2R locus. For instance, in A549 tumor cell line, the EEV-SC of a variant chimeric poxvirus defective in the J2R locus is higher than the EEV-SC measured in the same conditions and at the same time post-infection of at least one of the variant parental COP defective in the J2R locus and of the variant parental RPX defective in the J2R locus. In a more preferred embodiment, in A549 tumor cell line, the EEV-SC of a variant chimeric poxvirus defective in the J2R locus is higher than the EEV-SC measured in the same conditions and at the same time post-infection of at least three, preferably at least four, more preferably at least five, and even more preferably all six of the variant parental poxvirus strains RPX, CPX, COP, WY, WR and MVA all defective in the J2R locus.

Preferably, the EEV-SC of a variant chimeric poxvirus defective in the J2R locus is:
a) 16 hours post-injection in A549 tumor cell line, at least 1.2 times higher than the EEV-SC measured in the same conditions and at the same time post-infection of at least one of the variant parental poxvirus strains RPX, CPX, COP, WY, WR and MVA all defective in the J2R locus, preferably than that of at least one of the variant parental COP defective in the J2R locus or the variant parental RPX defective in the J2R locus, more preferably than that of at least two of the variant parental poxvirus strains RPX, CPX, COP, WY, WR and MVA all defective in the J2R locus, even more preferably than that of one of the variant parental COP defective in the J2R locus and of the variant parental RPX defective in the J2R locus, even more preferably than that of at least three, preferably at least four, more preferably at least five, and even more preferably all six of the variant parental poxvirus strains RPX, CPX, COP, WY, WR and MVA all defective in the J2R locus; and/or
b) 24 hours post-injection in A549 tumor cell line, at least 3 times higher than the EEV-SC measured in the same conditions and at the same time post-infection of at least one of the variant parental poxvirus strains RPX, CPX, COP, WY, WR and MVA all defective in the J2R locus, preferably than that of at least one of the variant parental COP defective in the J2R locus or the variant parental RPX defective in the J2R locus, more preferably than that of at least two of the variant parental poxvirus strains RPX, CPX, COP, WY, WR and MVA all defective in the J2R locus, even more preferably than that of one of the variant parental COP defective in the J2R locus and of the variant parental RPX defective in the J2R locus, even more preferably than that of at least three, preferably at least four, more preferably at least five, and even more preferably all six of the variant parental poxvirus strains RPX, CPX, COP, WY, WR and MVA all defective in the J2R locus.

For at least one producer cell (preferably a tumor cell), the EEV-SC of a variant chimeric poxvirus defective in the J2R locus may further be higher than the EEV-SC measured in the same conditions and at the same time post-infection of the Vaccinia virus strain IHD-J.

Preferably, the EEV-SC of the variant chimeric poxvirus defective in the J2R locus is at least:
a) 2 times higher than the EEV-SC measured in the same conditions and at the same time post-infection of Vaccinia virus strain IHD-J, 16 hours post-injection on A549 tumor cell line; and/or
b) 3 times higher than the EEV-SC measured in the same conditions and at the same time post-infection of Vaccinia virus strain IHD-J, 24 hours post-injection on A549 tumor cell line.

In a more specific embodiment of this aspect, the EEV-SC of the variant chimeric poxvirus defective in the J2R locus is:
a) at least 4%, preferably at least 5%, more preferably at least 6%, 16 hours post-infection of the said variant chimeric poxvirus defective in the J2R locus on A549 tumor cell line at an MOI of 10⁻² to 1, preferably at an MOI of 0.1; and/or
b) at least 5%, at least 6%, at least 7%, at least 8%, preferably at least 9%, more preferably at least 10%, even more preferably at least 11%, 24 hours post-infection of the said variant chimeric poxvirus defective in the J2R locus on A549 tumor cell line at an MOI of 10⁻² to 1, preferably at an MOI of 0.1.

In a more specific embodiment of this aspect, the EEV-SC of the variant chimeric poxvirus defective in the J2R locus is:
a) comprised between 4% and 9%, more preferably between 6% and 8%, 16 hours post-infection of the said variant chimeric poxvirus defective in the J2R locus on A549 tumor cell line at an MOI of 10⁻² to 1, preferably at an MOI of 0.1; and/or
b) comprised between 5% and 15%, more preferably between 10% and 13%, 24 hours post-infection of the said variant chimeric poxvirus defective in the J2R locus on A549 tumor cell line at an MOI of 10⁻² to 1, preferably at an MOI of 0.1.

Alternatively or in combination, for at least one tumor, the spreading capacity of a variant chimeric poxvirus defective in the J2R locus is higher than the spreading capacity measured in the same conditions and at the same time post-infection of at least one of the variant parental poxvirus strains RPX, CPX, COP, WY, WR and MVA all defective in the J2R locus. Preferably, for at least one tumor, the spreading capacity of a variant chimeric poxvirus defective in the J2R locus is higher than the spreading capacity measured in the same conditions and at the same time post-infection of the variant parental COP defective in the J2R locus or the variant parental RPX defective in the J2R locus. More preferably, for at least one tumor, the spreading capacity of a variant chimeric poxvirus defective in the J2R locus is higher than the spreading capacity measured in the same conditions and at the same time post-infection of at least two of the variant parental poxvirus strains RPX, CPX, COP, WY, WR and MVA all defective in the J2R locus. For instance, for at least one tumor, the spreading capacity of a variant chimeric poxvirus defective in the J2R locus is higher than the spreading capacity measured in the same conditions and at the same time post-infection of the variant parental COP defective in the J2R locus and the variant parental RPX defective in the J2R locus. In a preferred embodiment, for at least one tumor, the spreading capacity of a variant chimeric poxvirus defective in the J2R locus is higher than the spreading capacity measured in the same conditions and at the same time post-infection of at least three, preferably at least four, more preferably at least five and even more preferably all six of the variant parental poxvirus strains RPX, CPX, COP, WY, WR and MVA all defective in the J2R locus.

Alternatively or in combination, for at least one poxvirus-specific antibody and tumor, the neutralization rate of a variant chimeric poxvirus defective in the J2R locus is lower than the neutralization rate measured in the same conditions and at the same time post-infection of at least one of the variant parental poxvirus strains RPX, CPX, COP, WY, WR and MVA all defective in the J2R locus. Preferably for at least one poxvirus-specific antibody and tumor, the neutralization rate of a variant chimeric poxvirus defective in the J2R locus is lower than the neutralization rate measured in the same conditions and at the same time post-infection of the variant parental COP defective in the J2R locus. More preferably, for at least one poxvirus-specific antibody and tumor, the neutralization rate of a variant chimeric poxvirus defective in the J2R locus is lower than the neutralization rate measured in the same conditions and at the same time post-infection of at least two, preferably at least three, more preferably at least four, even more preferably at least five, and even more preferably all six of the variant parental poxvirus strains RPX, CPX, COP, WY, WR and MVA all defective in the J2R locus.

For a given tumor and virus, the viral neutralization rate may generally be determined in vitro, 3 to 5 days post-infection of tumor cells with the virus at an MOI of 3×10⁻⁵ to 3.

In this embodiment, for at least one vaccinia virus-specific antibody and on HCT116, the neutralization rate of a variant chimeric poxvirus defective in the J2R locus is lower than the neutralization rate measured in the same conditions and at the same time post-infection of at least one of the variant parental poxvirus strains RPX, CPX, COP, WY, WR and MVA all defective in the J2R locus. Preferably for at least one vaccinia virus-specific antibody and on HCT116, the neutralization rate of a variant chimeric poxvirus defective in the J2R locus is lower than the neutralization rate measured in the same conditions and at the same time post-infection of the variant parental COP defective in the J2R locus. More preferably, for at least one vaccinia virus-specific antibody and on HCT116, the neutralization rate of a variant chimeric poxvirus defective in the J2R locus is lower than the neutralization rate measured in the same conditions and at the same time post-infection of at least two, preferably at least three, more preferably at least four, even more preferably at least five, and even more preferably all six of the variant parental poxvirus strains RPX, CPX, COP, WY, WR and MVA all defective in the J2R locus.

In a specific aspect of this embodiment, for at least one vaccinia virus-specific antibody and on HCT116, the neutralization rate of a variant chimeric poxvirus defective in the J2R locus is at least 60 times, more preferably at least 70 times lower than the neutralization rate measured in the same conditions and at the same time post-infection of at least one of the variant parental poxvirus strains RPX, CPX, COP, WY, WR and MVA all defective in the J2R locus, preferably than that of the variant parental COP defective in the J2R locus, more preferably than that of at least two, preferably at least three, more preferably at least four, even more preferably at least five, and even more preferably all six of the variant parental poxvirus strains RPX, CPX, COP, WY, WR and MVA all defective in the J2R locus.

In a more specific aspect of this embodiment, for at least one vaccinia virus-specific antibody and on HCT116, the neutralization rate of a variant chimeric poxvirus defective in the J2R locus is between 30 to 100 times, even more between 40 to 90 times, even more between 55 to 75 times lower than the neutralization rate measured in the same conditions and at the same time post-infection of at least one of the variant parental poxvirus strains RPX, CPX, COP, WY, WR and MVA all defective in the J2R locus, preferably than that of the variant parental COP defective in the J2R locus, more preferably than that of at least two, preferably at least three, more preferably at least four, even more preferably at least five, and even more preferably all six of the variant parental poxvirus strains RPX, CPX, COP, WY, WR and MVA all defective in the J2R locus.

Alternatively or in combination, the complement-mediated virus neutralization rate of a variant chimeric poxvirus defective in the J2R locus is lower than the complement-mediated virus neutralization rate measured in the same conditions of at least one of the variant parental poxvirus strains RPX, CPX, COP, WY, WR and MVA all defective in the J2R locus. Preferably, the complement-mediated virus neutralization rate of a variant chimeric poxvirus defective in the J2R locus is lower than the complement-mediated virus neutralization rate measured in the same conditions of the variant parental COP defective in the J2R locus. More preferably, the complement-mediated virus neutralization rate of a variant chimeric poxvirus defective in the J2R locus is lower than the complement-mediated virus neutralization rate measured in the same conditions of at least two, preferably at least three, more preferably at least four, even more preferably at least five, and even more preferably all six of the variant parental poxvirus strains RPX, CPX, COP, WY, WR and MVA all defective in the J2R locus.

For a given virus, the complement-mediated virus neutralization rate may be generally determined in vitro, in presence of active or heat-inactivated serum with the virus at a dose of 10⁴ to 10⁸ PFU/mL.

In a specific aspect of this embodiment, the complement-mediated virus neutralization rate of a variant chimeric poxvirus defective in the J2R locus is at least 5 times, more preferably at least 6 times, even more preferably at least 6,8 times lower than the complement-mediated virus neutralization rate measured in the same conditions of at least one of the variant parental poxvirus strains RPX, CPX, COP, WY, WR and MVA all defective in the J2R locus, preferably than that of the variant parental COP defective in the J2R locus, more preferably than that of at least two, preferably at least three, more preferably at least four, even more preferably at least five, and even more preferably all six of the variant parental poxvirus strains RPX, CPX, COP, WY, WR and MVA all defective in the J2R locus.

In a more specific aspect of this embodiment, the complement-mediated virus neutralization rate of a variant chimeric poxvirus defective in the J2R locus is between 5 to 10 times, even more between 6 to 8 times lower than the complement-mediated virus neutralization rate measured in the same conditions of at least one of the variant parental poxvirus strains RPX, CPX, COP, WY, WR and MVA all defective in the J2R locus, preferably than that of the variant parental COP defective in the J2R locus, more preferably than that of at least two, preferably at least three, more preferably at least four, even more preferably at least five, and even more preferably all six of the variant parental poxvirus strains RPX, CPX, COP, WY, WR and MVA all defective in the J2R locus.

Alternatively or in combination, for at least one producer cell (preferably in tumor cell), the syncytia formation capacity of a variant chimeric poxvirus defective in the J2R locus is higher than the syncytia formation capacity measured in the same conditions and at the same time post-infection of at least one of the variant parental poxvirus strains RPX, CPX, COP, WY, WR and MVA all defective in the J2R locus. Preferably, for at least one producer cell (preferably in tumor cell), the syncytia formation capacity of a variant chimeric poxvirus defective in the J2R locus is higher than the syncytia formation capacity measured in the same conditions and at the same time post-infection of the variant parental COP defective in the J2R locus. More preferably, for at least one producer cell (preferably a tumor cell), the syncytia formation capacity of a variant chimeric poxvirus defective in the J2R locus is higher than the syncytia formation capacity measured in the same conditions and at the same time post-infection of at least two of the variant parental poxvirus strains RPX, CPX, COP, WY, WR and MVA all defective in the J2R locus. For instance, for at least one producer cell, the syncytia formation capacity of a variant chimeric poxvirus defective in the J2R locus is higher than the syncytia formation capacity measured in the same conditions and at the same time post-infection of at least three, preferably at least four, more preferably at least five and even more preferably all six of the variant parental poxvirus strains RPX, CPX, COP, WY, WR and MVA all defective in the J2R locus.

In a specific aspect of this embodiment, in HCT116, the syncytia formation capacity of a variant chimeric poxvirus defective in the J2R locus is higher than the syncytia formation capacity measured in the same conditions and at the same time post-infection of at least one of the variant parental poxvirus strains RPX, CPX, COP, WY, WR and MVA all defective in the J2R locus. Preferably, in HCT116, the syncytia formation capacity of a variant chimeric poxvirus defective in the J2R locus is higher than the syncytia formation capacity measured in the same conditions and at the same time post-infection of the variant parental COP defective in the J2R locus. More preferably, in HCT116, the syncytia formation capacity of a variant chimeric poxvirus defective in the J2R locus is higher than the syncytia formation capacity measured in the same conditions and at the same time post-infection of at least two of the variant parental poxvirus strains RPX, CPX, COP, WY, WR and MVA all defective in the J2R locus. For instance, for at least one producer cell, the syncytia formation capacity of a variant chimeric poxvirus defective in the J2R locus is higher than the syncytia formation capacity measured in the same conditions and at the same time post-infection of at least three, preferably at least four, more preferably at least five and even more preferably all six of the variant parental poxvirus strains RPX, CPX, COP, WY, WR and MVA all defective in the J2R locus.

A variant chimeric poxvirus defective in the J2R locus according to the invention may combine several of the above-described functional features, including:
a high oncolytic power as disclosed above (preferably compared to variant parental Vaccinia virus strain Copenhagen (COP) defective in the J2R locus, to variant parental Rabbitpox virus strain Utrecht (RPX) defective in the J2R locus, to variant parental COP defective in the J2R locus and variant parental RPX defective in the J2R locus, or to all variant parental poxvirus strains defective in the J2R locus);
a low viral replication in healthy cells (preferably primary cells) as disclosed above (preferably compared to variant parental Vaccinia virus strain Copenhagen (COP) defective in the J2R locus or to all variant parental poxvirus strains defective in the J2R locus);
a high EEV-SC, a high syncytia formation capacity, a high spreading capacity, a low neutralization rate in the presence of poxvirus-specific antibody, and/or a low complement-mediated virus neutralization rate as disclosed above (preferably compared to variant parental Vaccinia virus strain Copenhagen (COP) defective in the J2R locus, to variant parental Rabbitpox virus strain Utrecht (RPX) defective in the J2R locus, to Vaccinia virus strain IHD-J or to all variant parental poxvirus strains defective in the J2R locus).

In particular, a variant chimeric poxvirus defective in the J2R locus may comprise:
a high oncolytic power as disclosed above and a low viral replication in healthy cells (preferably primary cells) as disclosed above;
a high oncolytic power as disclosed above and (a high EEV-SC, a high syncytia formation capacity, a high spreading capacity, a low neutralization rate in the presence of poxvirus-specific antibody, and/or a low complement-mediated virus neutralization rate as disclosed above);
a low viral replication in healthy cells (preferably primary cells) as disclosed above and (a high EEV-SC, a high syncytia formation capacity, a high spreading capacity, a low neutralization rate in the presence of poxvirus-specific antibody, and/or a low complement-mediated virus neutralization rate as disclosed above); or
a high oncolytic power as disclosed above, a low viral replication in healthy cells (preferably primary cells) as disclosed above and (a high EEV-SC, a high syncytia formation capacity, a high spreading capacity, a low neutralization rate in the presence of poxvirus-specific antibody, and/or a low complement-mediated virus neutralization rate as above).

A particularly preferred variant chimeric poxvirus defective in the J2R locus according to the invention comprises the following functional features:
for at least one tumor, the oncolytic power of said variant chimeric poxvirus defective in the J2R locus is higher than the oncolytic power measured in the same conditions and at the same time post-infection of the variant parental Vaccinia virus strain Copenhagen (COP) defective in the J2R locus or the variant parental Rabbitpox virus strain Utrecht (RPX) defective in the J2R locus;
for at least one healthy cell (preferably primary cell), the viral replication in the healthy cell (preferably primary cell) of said variant chimeric poxvirus defective in the J2R locus is lower than that of the variant parental Vaccinia virus strain Copenhagen (COP) defective in the J2R locus;
for at least one producer cell (preferably a tumor cell), the EEV-SC of said variant chimeric poxvirus defective in the J2R locus is higher than the EEV-SC measured in the same conditions and at the same time post-infection of the variant parental Rabbitpox virus strain Utrecht (RPX) defective in the J2R locus or the Vaccinia virus strain IHD-J;
optionally, for at least one poxvirus-specific antibody and tumor, the neutralization rate of said variant chimeric poxvirus defective in the J2R locus is lower than the neutralization rate measured in the same conditions and at the same time post-infection of the variant parental Vaccinia virus strain Copenhagen (COP) defective in the J2R locus;
optionally, the complement-mediated virus neutralization rate of said variant chimeric poxvirus defective in the J2R locus is lower than the complement-mediated virus neutralization rate measured in the same conditions of the parental Vaccinia virus strain Copenhagen (COP) defective in J2R locus; and
optionally, for at least one producer cell (preferably a tumor cell), the syncytia formation capacity of said variant chimeric poxvirus defective in J2R locus is higher than the syncytia formation capacity measured in the same conditions and at the same time post-infection of the parental Vaccinia virus strain Copenhagen (COP) defective in J2R locus.

A further particularly preferred variant chimeric poxvirus defective in the J2R locus according to the invention comprises the following functional features:
for at least one tumor, the oncolytic power of said variant chimeric poxvirus defective in the J2R locus is higher than the oncolytic power measured in the same conditions and at the same time post-infection of the variant parental Vaccinia virus strain Copenhagen (COP) defective in the J2R locus or the variant parental Rabbitpox virus strain Utrecht (RPX) defective in the J2R locus;
for at least one healthy cell (preferably primary cell), the viral replication in the healthy cell (preferably primary cell) of said variant chimeric poxvirus defective in the J2R locus is lower than that of the variant parental Vaccinia virus strain Copenhagen (COP) defective in the J2R locus;
for at least one producer cell (preferably a tumor cell), the syncytia formation capacity of said variant chimeric poxvirus defective in J2R locus is higher than the syncytia formation capacity measured in the same conditions and at the same time post-infection of the parental Vaccinia virus strain Copenhagen (COP) defective in J2R locus;
optionally, for at least one producer cell (preferably a tumor cell), the EEV-SC of said variant chimeric poxvirus defective in the J2R locus is higher than the EEV-SC measured in the same conditions and at the same time post-infection of the variant parental Rabbitpox virus strain Utrecht (RPX) defective in the J2R locus or the Vaccinia virus strain IHD-J;
optionally, for at least one poxvirus-specific antibody and tumor, the neutralization rate of said variant chimeric poxvirus defective in the J2R locus is lower than the neutralization rate measured in the same conditions and at the same time post-infection of the variant parental Vaccinia virus strain Copenhagen (COP) defective in the J2R locus; and
optionally, the complement-mediated virus neutralization rate of said variant chimeric poxvirus defective in the J2R locus is lower than the complement-mediated virus neutralization rate measured in the same conditions of the parental Vaccinia virus strain Copenhagen (COP) defective in J2R locus.

A more particularly preferred variant chimeric poxvirus defective in the J2R locus according to the invention comprises the following functional features:
for at least one tumor, the oncolytic power of said variant chimeric poxvirus defective in the J2R locus is higher than the oncolytic power measured in the same conditions and at the same time post-infection of the variant parental Vaccinia virus strain Copenhagen (COP) defective in the J2R locus or the variant parental Rabbitpox virus strain Utrecht (RPX) defective in the J2R locus;
for at least one healthy cell (preferably primary cell), the viral replication in the healthy cell (preferably primary cell) of said variant chimeric poxvirus defective in the J2R locus is lower than that of the variant parental Vaccinia virus strain Copenhagen (COP) defective in the J2R locus;
for at least one producer cell (preferably a tumor cell), the EEV-SC of said variant chimeric poxvirus defective in the J2R locus is higher than the EEV-SC measured in the same conditions and at the same time post-infection of the variant parental Rabbitpox virus strain Utrecht (RPX) defective in the J2R locus or the Vaccinia virus strain IHD-J;
for at least one producer cell (preferably a tumor cell), the syncytia formation capacity of said variant chimeric poxvirus defective in J2R locus is higher than the syncytia formation capacity measured in the same conditions and at the same time post-infection of the parental Vaccinia virus strain Copenhagen (COP) defective in J2R locus;
optionally, for at least one poxvirus-specific antibody and tumor, the neutralization rate of said variant chimeric poxvirus defective in the J2R locus is lower than the neutralization rate measured in the same conditions and at the same time post-infection of the variant parental Vaccinia virus strain Copenhagen (COP) defective in the J2R locus; and
optionally, the complement-mediated virus neutralization rate of said variant chimeric poxvirus defective in the J2R locus is lower than the complement-mediated virus neutralization rate measured in the same conditions of the parental Vaccinia virus strain Copenhagen (COP) defective in J2R locus.

The variant chimeric poxvirus defective in the J2R locus of the invention may alternatively comprise a high therapeutic index as disclosed above and (a high EEV-SC, a high syncytia formation capacity, a high spreading capacity, a low neutralization rate in the presence of poxvirus-specific antibody and/or a low complement-mediated virus neutralization rate as disclosed above). A particularly preferred variant chimeric poxvirus defective in the J2R locus according to the invention comprises the following functional features:
for at least one organ, the therapeutic index of said variant chimeric poxvirus defective in the J2R locus is higher than the therapeutic index measured in the same conditions and at the same time post-infection of the variant parental Vaccinia virus strain Copenhagen (COP) defective in the J2R locus;
for at least one producer cell (preferably a tumor cell), the EEV-SC of said variant chimeric poxvirus defective in the J2R locus is higher than the EEV-SC measured in the same conditions and at the same time post-infection of the variant parental Rabbitpox virus strain Utrecht (RPX) defective in the J2R locus or the Vaccinia virus strain IHD-J;
optionally, for at least one poxvirus-specific antibody and tumor, the neutralization rate of said variant chimeric poxvirus defective in the J2R locus is lower than the neutralization rate measured in the same conditions and at the same time post-infection of the variant parental Vaccinia virus strain Copenhagen (COP) defective in the J2R locus;
optionally, the complement-mediated virus neutralization rate of said variant chimeric poxvirus defective in J2R locus is lower than the complement-mediated virus neutralization rate measured in the same conditions of the parental Vaccinia virus strain Copenhagen (COP) defective in J2R locus; and
optionally, for at least one producer cell (preferably a tumor cell), the syncytia formation capacity of said variant chimeric poxvirus defective in J2R locus is higher than the syncytia formation capacity measured in the same conditions and at the same time post-infection of the parental Vaccinia virus strain Copenhagen (COP) defective in J2R locus.

A further preferred variant chimeric poxvirus defective in the J2R locus according to the invention comprises the following functional features:
for at least one organ, the therapeutic index of said variant chimeric poxvirus defective in the J2R locus is higher than the therapeutic index measured in the same conditions and at the same time post-infection of the variant parental Vaccinia virus strain Copenhagen (COP) defective in the J2R locus;
for at least one producer cell (preferably a tumor cell), the syncytia formation capacity of said variant chimeric poxvirus defective in J2R locus is higher than the syncytia formation capacity measured in the same conditions and at the same time post-infection of the parental Vaccinia virus strain Copenhagen (COP) defective in J2R locus;
optionally, for at least one producer cell (preferably a tumor cell), the EEV-SC of said variant chimeric poxvirus defective in the J2R locus is higher than the EEV-SC measured in the same conditions and at the same time post-infection of the variant parental Rabbitpox virus strain Utrecht (RPX) defective in the J2R locus or the Vaccinia virus strain IHD-J;
optionally, for at least one poxvirus-specific antibody and tumor, the neutralization rate of said variant chimeric poxvirus defective in the J2R locus is lower than the neutralization rate measured in the same conditions and at the same time post-infection of the variant parental Vaccinia virus strain Copenhagen (COP) defective in the J2R locus; and
optionally, the complement-mediated virus neutralization rate of said variant chimeric poxvirus defective in J2R locus is lower than the complement-mediated virus neutralization rate measured in the same conditions of the parental Vaccinia virus strain Copenhagen (COP) defective in J2R locus.

A more particularly preferred variant chimeric poxvirus defective in the J2R locus according to the invention comprises the following functional features:
for at least one organ, the therapeutic index of said variant chimeric poxvirus defective in the J2R locus is higher than the therapeutic index measured in the same conditions and at the same time post-infection of the variant parental Vaccinia virus strain Copenhagen (COP) defective in the J2R locus;
for at least one producer cell (preferably a tumor cell), the EEV-SC of said variant chimeric poxvirus defective in the J2R locus is higher than the EEV-SC measured in the same conditions and at the same time post-infection of the variant parental Rabbitpox virus strain Utrecht (RPX) defective in the J2R locus or the Vaccinia virus strain IHD-J;
for at least one producer cell (preferably a tumor cell), the syncytia formation capacity of said variant chimeric poxvirus defective in J2R locus is higher than the syncytia formation capacity measured in the same conditions and at the same time post-infection of the parental Vaccinia virus strain Copenhagen (COP) defective in J2R locus;
optionally, for at least one poxvirus-specific antibody and tumor, the neutralization rate of said variant chimeric poxvirus defective in the J2R locus is lower than the neutralization rate measured in the same conditions and at the same time post-infection of the variant parental Vaccinia virus strain Copenhagen (COP) defective in the J2R locus; and
optionally, the complement-mediated virus neutralization rate of said variant chimeric poxvirus defective in J2R locus is lower than the complement-mediated virus neutralization rate measured in the same conditions of the parental Vaccinia virus strain Copenhagen (COP) defective in J2R locus.

Any variant chimeric poxvirus defective in the J2R locus combining several functional features as described above may further have at least 99%, at least 99,1%, at least 99,2%, at least 99,3%, at least 99,4%, at least 99,5%, at least 99,6%, at least 99,7%, at least 99,8%, at least 99,9%, at least 99,91%, at least 99,92%, at least 99,93%, at least 99,94%, at least 99,95%, at least 99,96%, at least 99,97%, at least 99,98%, at least 99,99%, or even 100% of identity with SEQ ID NO:8.

Variant Chimeric Poxvirus Defective in the 14L and/or F4L Locus

In another embodiment, the variant chimeric poxvirus of the invention is defective in at least one of the ribonucleotide reductase (RR) loci, where are located the RR encoding-genes: 14L encoding-gene (similar to 14L gene in COP) and F4L encoding-gene (similar to F4L gene in COP). In the natural context, the ribonucleotide reductase enzyme catalyzes the reduction of ribonucleotides to deoxyribonucleotides that represents a crucial step in DNA biosynthesis. The viral enzyme is similar in subunit structure to the mammalian enzyme, being composed of two heterologous subunits, designed 14L and F4L. In the context of the invention, either the locus encoding the R1 large subunit, or the locus encoding the R2 small subunit, or both may be defective.

In an embodiment, the variant chimeric poxvirus of the invention is defective in the 14L locus. Alternatively, the variant chimeric poxvirus of the invention is defective in the F4L locus, or in both the 14L and the F4L loci.

Variant Chimeric Poxvirus Defective in the J2R Locus and Defective in the 14L and/or F4L Locus

In a preferred embodiment, the variant chimeric poxvirus of this invention may be defective in the J2R locus and in one or both of the 14L and F4L loci. Such a double deleted variant chimeric poxvirus is defective for both TK and RR activities (e.g.: as described in WO2009/065546 and Foloppe et al., 2008, Gene Ther., 15:1361-1371). Double J2R and one or both of 14L and F4L deleted variant chimeric poxviruses are thus particularly advantageous due to low replication in healthy cells (preferably primary cells) and higher therapeutic index than the corresponding wild-type chimeric poxviruses.

Preferably, the variant chimeric poxvirus has at least 99%, at least 99,1%, at least 99,2%, at least 99,3%, at least 99,4%, at least 99,5%, at least 99,6%, at least 99,7%, at least 99,8%, at least 99,9%, at least 99,91%, at least 99,92%, at least 99,93%, at least 99,94%, at least 99,95%, at least 99,96%, at least 99,97%, at least 99,98%, at least 99,99%, or even 100% of identity with SEQ ID NO:9. SEQ ID NO:9 is the sequence of the core region and one of the ITRs of POXSTG19730, which corresponds to POXSTG19503, except that the GFP::FCU1 sequence has been inserted into the J2R locus and mCherry gene under the control of the pH5R promoter has been inserted into the 14L locus.

More preferably, the variant chimeric poxvirus has at least 99%, at least 99,1%, at least 99,2%, at least 99,3%, at least 99,4%, at least 99,5%, at least 99,6%, at least 99,7%, at least 99,8%, at least 99,9%, at least 99,91%, at least 99,92%, at least 99,93%, at least 99,94%, at least 99,95%, at least 99,96%, at least 99,97%, at least 99,98%, at least 99,99%, or even 100% of identity with SEQ ID NO: 10. SEQ ID NO:10 is the sequence of the core region and one of the ITRs of POXSTG20150, which corresponds to POXSTG19503 after the deletion of J2R gene sequence encoding TK protein and 14L gene sequence encoding for the large subunit of the RR protein.

Even more preferably, the variant chimeric poxvirus is strain POXSTG19503 having accession number CNCM-I-5913 and is defective in the J2R locus and defective in the 14L and/or F4L locus.

Alternatively, or in combination, a variant chimeric poxvirus defective in the J2R and one or both of the 14L and F4L loci (preferably the 14L locus) preferably comprises the following functional features or combinations of functional features:
a) for at least one tumor, the oncolytic power of a variant chimeric poxvirus defective in the J2R locus and in one or both of the 14L and F4L loci according to the invention is higher than the oncolytic power measured in the same conditions and at the same time post-infection of at least one (preferably the variant oncolytic parental COP defective in the J2R locus and in one or both of the 14L and F4L loci), at least two, at least three, at least four or all five of the variant oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) defective in the J2R locus and in one or both of the 14L and F4L loci;
b) for at least one healthy cell (preferably primary cell), the viral replication in the healthy cell (preferably primary cell) of the variant chimeric poxvirus defective in the J2R locus and in one or both of the 14L and F4L loci is lower than that of at least one (preferably the variant oncolytic parental COP defective in the J2R locus and in one or both of the 14L and F4L loci), at least two, at least three, at least four or all five of the variant oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus and in one or both of the 14L and F4L loci;
c) for at least one producer cell (preferably a tumor cell), the EEV-SC of a variant chimeric poxvirus defective in the J2R locus and in one or both of the 14L and F4L loci is higher than the EEV-SC measured in the same conditions and at the same time post-infection of at least one (preferably the variant oncolytic parental COP defective in the J2R locus and in one or both of the 14L and F4L loci), at least two, at least three, at least four, at least five or all six of the variant parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia Virus Ankara (MVA) all defective in the J2R locus and in one or both of the 14L and F4L loci;
d) for at least one tumor, the spreading capacity of a variant chimeric poxvirus defective in the J2R locus and in one or both of the 14L and F4L loci is higher than the spreading capacity measured in the same conditions and at the same time post-infection of at least one (preferably the variant oncolytic parental COP defective in the J2R locus and in one or both of the 14L and F4L loci), at least two, at least three, at least four, at least five or all six of the variant parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia Virus Ankara (MVA) all defective in the J2R locus and in one or both of the 14L and F4L loci;
e) for at least one poxvirus-specific antibody and tumor, the neutralization rate of a variant chimeric poxvirus defective in the J2R locus and in one or both of the 14L and F4L loci is lower than the neutralization rate measured in the same conditions and at the same time post-infection of at least one (preferably the variant oncolytic parental COP defective in the J2R locus and in one or both of the 14L and F4L loci), at least two, at least three, at least four, at least five or all six of the variant parental poxvirus strains RPX, CPX, COP, WY, WR and MVA all defective in the J2R locus and in one or both of the 14L and F4L loci;
f) the complement-mediated virus neutralization rate of a variant chimeric poxvirus defective in the J2R locus and in one or both of the 14L and F4L loci is lower than the complement-mediated virus neutralization rate measured in the same conditions of at least one (preferably the variant oncolytic parental COP defective in the J2R locus and in one or both of the 14L and F4L loci), at least two, at least three, at least four, at least five or all six of the variant parental poxvirus strains RPX, CPX, COP, WY, WR and MVA all defective in the J2R locus and in one or both of the 14L and F4L loci;
g) for at least one producer cell (preferably a tumor cell), the syncytia formation capacity of a variant chimeric poxvirus defective in the J2R locus and in one or both of the 14L and F4L loci is higher than the syncytia formation capacity measured in the same conditions and at the same time post-infection of at least one (preferably the variant oncolytic parental COP defective in the J2R locus and in one or both 14L and F4L loci), at least two, at least three, at least four, at least five or at least six of the variant parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia Virus Ankara (MVA) all defective in the J2R locus and in one or both of the 14L and F4L loci;
h) a) and b);
i) a) and (c) or d) or e) or f) or g));
j) a), b) and (c) or d) or e) or f) or g)), preferably a), b) and c), or a), b), and g);
k) a), b), c) and (e) or f) or g)), preferably a), b), c) and f); or a), b), c) and e);
l) a), b), g) and (e) or f));
m) a), b), c), e) and (f) or g));
n) a), b), c), f) and g);
o) a), b), e), f) and g);
p) a), b), c), e), f) and g), and
q) any combination thereof.

Alternatively, a variant chimeric poxvirus defective in the J2R and one or both of the 14L and F4L loci (preferably the 14L locus) preferably comprises the following functional features or combinations of functional features:
a) for at least one organ, the therapeutic index of said variant chimeric poxvirus defective in the J2R locus and in one or both of the 14L and F4L loci is higher than the therapeutic index measured in the same conditions and at the same time post-infection of at least one (preferably the variant oncolytic parental COP defective in the J2R locus and in one or both of the 14L and F4L loci), at least two, at least three, at least four, at least five or all six of the variant parental oncolytic poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) all defective in the J2R locus and in one or both of the 14L and F4L loci;
b) for at least one producer cell (preferably a tumor cell), the EEV-SC of a variant chimeric poxvirus defective in the J2R locus and in one or both of the 14L and F4L loci is higher than the EEV-SC measured in the same conditions and at the same time post-infection of at least one (preferably the variant oncolytic parental COP defective in the J2R locus and in one or both of the 14L and F4L loci), at least two, at least three, at least four, at least five or all six of the variant parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia Virus Ankara (MVA) all defective in the J2R locus and in one or both of the 14L and F4L loci;
c) for at least one tumor, the spreading capacity of a variant chimeric poxvirus defective in the J2R locus and in one or both of the 14L and F4L loci is higher than the spreading capacity measured in the same conditions and at the same time post-infection of at least one (preferably the variant oncolytic parental COP defective in the J2R locus and in one or both of the 14L and F4L loci), at least two, at least three, at least four, at least five or all six of the variant parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia Virus Ankara (MVA) all defective in the J2R locus and in one or both of the 14L and F4L loci;
d) for at least one poxvirus-specific antibody and tumor, the neutralization rate of a variant chimeric poxvirus defective in the J2R locus and in one or both of the 14L and F4L loci is lower than the neutralization rate measured in the same conditions and at the same time post-infection of at least one (preferably the variant oncolytic parental COP defective in the J2R locus and in one or both of the 14L and F4L loci), at least two, at least three, at least four, at least five or all six of the variant parental poxvirus strains RPX, CPX, COP, WY, WR and MVA all defective in the J2R locus and in one or both of the 14L and F4L loci;
e) the complement-mediated virus neutralization rate of a variant chimeric poxvirus defective in the J2R locus and in one or both of the 14L and F4L loci is lower than the complement-mediated virus neutralization rate measured in the same conditions of at least one (preferably the variant oncolytic parental COP defective in the J2R locus and in one or both of the 14L and F4L loci), at least two, at least three, at least four, at least five or all six of the variant parental poxvirus strains RPX, CPX, COP, WY, WR and MVA all defective in the J2R locus and in one or both of the 14L and F4L loci;
f) for at least one producer cell (preferably a tumor cell), the syncytia formation capacity of a variant chimeric poxvirus defective in the J2R locus and in one or both of the 14L and F4L loci is higher than the syncytia formation capacity measured in the same conditions and at the same time post-infection of at least one (preferably the variant oncolytic parental COP defective in the J2R locus and in one or both of the 14L and F4L loci), at least two, at least three, at least four, at least five or all six of the variant parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR) and Modified Vaccinia Virus Ankara (MVA) all defective in the J2R locus and in one or both of the 14L and F4L loci;
g) a) and (b) or c) or d) or e) or f)), preferably a) and b);
h) a), b) and (d) or e) or f)), preferably a), b) and d), or a), b) and e), or a), b) and f);
i) a), d) and (e) or f));
j) a), e) and f);
k) a), b), d) and (e) or f));
I) a), b), d) and e);
m) a), b), e) and f);
n) a), d), e) and f);
o) a), b), d), e), and f); and
p) any combination thereof.
   Further Viral Genes that May be Altered in the Variant Chimeric Poxvirus of the Invention

Alternatively, or in combination with deficiency in the J2R locus, in one or both of the 14L and F4L loci, or in the J2R locus and in one or both of the 14L and F4L loci, the variant chimeric poxvirus of the invention may be further defective in the M2L locus (preference for modification leading to a suppressed expression of the viral m2 protein, such as M2L locus deletion).

In one embodiment, the variant chimeric poxvirus is defective in the J2R locus (preference for modification resulting in a suppressed expression of the viral TK protein) and in the M2L locus (preference for modification leading to a suppressed expression of the viral m2 protein), resulting in a variant chimeric poxvirus defective for both m2 and TK functions (m2-tk-variant chimeric poxvirus). Partial or complete deletion of said M2L locus and/or J2R locus as well as insertion of foreign nucleic acid in the M2L locus and/or J2R locus are contemplated in the context of the present invention to inactivate m2 and tk functions.

In another embodiment the variant chimeric poxvirus is defective in one or both or the 14L and F4L loci (preference for modification leading to a suppressed expression of the viral ribonucleotide reductase (RR) protein) and in the M2L locus (preference for modification leading to a suppressed expression of the viral m2 protein), resulting in a chimeric poxvirus defective for both m2 and rr functions (m2 and rr-defective variant chimeric poxvirus). In the context of the invention, the variant chimeric poxvirus can be modified either in the 14L gene (encoding the R1 large subunit) or in the F4L gene (encoding the R2 small subunit) or both to provide a RR-defective variant chimeric poxvirus. E.g.: by partial or complete deletion of said 14L and/or F4L locus/loci.

In another embodiment, the variant chimeric poxvirus is defective in the J2R locus, in one or both of the 14L and F4L loci, and in the M2L locus (triple defective virus with modifications in the M2L, J2R and 14L loci; M2L, J2R and F4L loci or M2L, J2R, 14L and F4L loci), resulting in a variant chimeric poxvirus defective for M2, TK and RR activities (m2-, tk-, rr-variant chimeric poxvirus).

Alternatively, or in combination with alteration of one or more of the J2R locus, the 14L and/or F4L locus, and the M2 locus, the variant chimeric poxvirus of this invention may be defective for dUTPase resulting from alteration of the dUTPase encoding-gene (similar to F2L gene in COP).

Alternatively, or in combination, other strategies may also be pursued to further increase the virus tumor-specificity. A representative example of suitable modification includes disruption of the hemagglutinin encoding-gene (similar to A56R gene in COP), optionally in combination with J2R deletion (Zhang et al., 2007, Cancer Res. 67:10038-46). Disruption of interferon modulating gene(s) may also be advantageous (similar to B8R or B18R genes in COP) or the caspase-1 inhibitor (similar to B13R gene in COP). Another suitable modification comprises the disruption of the gene encoding the viral dUTPase involved in both maintaining the fidelity of DNA replication and providing the precursor for the production of TMP by thymidylate synthase (Broyles et al., 1993, Virol. 195:863-5).

### Recombinant Chimeric Poxvirus Encoding Heterologous Nucleic Acid(S) of Interest

Any chimeric poxvirus of the invention (either wild-type or variant as described above) may further comprise one or more heterologous nucleic acid(s) of interest inserted in its genome. The invention thus also provides a recombinant (either wild-type or variant as described above) chimeric poxvirus, further comprising one or more heterologous nucleic acid(s) of interest inserted in its genome.

According to the invention, the heterologous nucleic acid(s) of interest can originate from Prokaryotes (comprising the kingdoms of Bacteria, Archaea), Acaryotes (comprising the viruses) or

Eukaryotes (comprising the kingdoms of Protista, Fungi, Plantae, Animalia). Advantageously, said nucleic acid of interest encodes all or part of a polypeptide. A polypeptide is understood to be any translational product of a polynucleotide regardless of size, and whether glycosylated or not, and includes peptides and proteins.

In one embodiment, the nucleic acid of interest encodes a polypeptide of therapeutic interest, which is capable of providing a biological activity when administered appropriately to a subject or which is expected to cause a beneficial effect on the course or a symptom of the pathological condition to be treated. A vast number of nucleic acids of interest may be envisaged in the context of the invention such as those encoding polypeptides that can compensate for defective or deficient proteins in the subject, or those that act through toxic effects to limit or remove harmful cells from the body or those that encode immunity conferring polypeptides. They may be native or obtained from the latter by mutation, deletion, substitution and/or addition of one or more nucleotides. Representative examples of suitable polypeptides of therapeutic interest include, without limitation, polypeptides capable of potentiating anti-tumor efficacy (such as immunostimulatory polypeptides), as well as antigens for inducing or activating an immune humoral and/or cellular response, suicide polypeptides which are capable of reinforcing the oncolytic nature of the chimeric poxvirus of the present invention, or permease to increase the cellular nucleoside or nucleotide pool among many others. The present invention also encompasses chimeric poxvirus expressing two or more polypeptides of interest as described herein, e.g.: at least two antigens, at least one antigen and one cytokine, at least two antigens and one cytokine, etc.

### Immunostimulatory Polypeptide

A specific embodiment of the invention is directed to a recombinant (wild-type or variant) chimeric poxvirus comprising an immunostimulatory polypeptide. As used herein, the term “immunostimulatory polypeptide” refers to a polypeptide, or protein, which has the ability to stimulate the immune system, in a specific or non-specific way. A vast number of proteins are known in the art for their ability to exert an immunostimulatory effect. Examples of suitable immunostimulatory proteins in the context of the invention include, without limitation, immune checkpoint inhibitors, including, but not limited to anti-PD1, anti-PDL1, anti-PDL-2, anti-CTLA4, anti-Tim3, anti-LAG3, anti-BTLA; cytokines, like alpha, beta or gamma interferon, interleukins or tumor necrosis factor; agents that affect the regulation of cell surface receptors such as, e.g. inhibitors of Epidermal Growth Factor Receptor (in particular cetuximab, panitumumab, zalutumumab, nimotuzumab, matuzumab, gefitinib, erlotinib or lapatinib) or inhibitors of Human Epidermal Growth Factor Receptor-2 (in particular trastuzumab); agents that affect angiogenesis such as, e.g. inhibitor of Vascular Endothelial Growth Factor (in particular bevacizumab or ranibizumab); agents that stimulate stem cells to produce granulocytes, macrophages such as, e.g. granulocyte macrophage-colony stimulating factor and B7 proteins. In a preferred embodiment, said nucleic acid of interest is a cytokine, more preferably an interleukin, even more preferably an IL-12.

In a preferred embodiment of the invention, a recombinant variant chimeric poxvirus is defective in the J2R locus and further encodes an interleukin. In a more preferred embodiment, a recombinant variant chimeric poxvirus is defective in the J2R locus and further encodes an IL-12.

In a preferred embodiment of the invention, a recombinant variant chimeric poxvirus is defective in the J2R and one or both of the F4L and 14L loci, and further encodes an interleukin. In a more preferred embodiment, a recombinant variant chimeric poxvirus is defective in the J2R and in one or both of the F4L and 14L loci and further encodes an IL-12.

### Antigens

Another embodiment of the invention is directed to a recombinant (wild-type or variant) chimeric poxvirus encoding an antigen. The term “antigen” generally refers to a substance that is recognized and selectively bound by an antibody or by a T cell antigen receptor, in order to trigger an immune response. It is contemplated that the term antigen encompasses native antigen as well as fragment (e.g.: epitopes, immunogenic domains, etc.) and analogue thereof, provided that such fragment or analogue is capable of being the target of an immune response. Suitable antigens in the context of the invention are preferably polypeptides (e.g.: peptides, polypeptides, post translationally modified polypeptides, etc.) including one or more B cell epitope(s) or one or more T cell epitope(s) or both B and T cell epitope(s) and capable of raising an immune response, preferably, a humoral or cell response that can be specific for that antigen. Typically, the one or more antigen(s) is selected in connection with the disease to treat. Preferred antigens for use herein are cancer antigens and antigens of tumor-inducing pathogens.

In certain embodiments, the antigen(s) encoded by the recombinant chimeric poxvirus is/are cancer antigen(s) (also called tumor-associated antigens) that is associated with and/or serve as markers for cancers. Cancer antigens encompass various categories of polypeptides, e.g. those which are normally silent (i.e. not expressed) in healthy cells (preferably primary cells), those that are expressed only at low levels or at certain stages of differentiation and those that are temporally expressed such as embryonic and fetal antigens as well as those resulting from mutation of cellular genes, such as oncogenes (e.g. activated ras oncogene), proto-oncogenes (e.g. ErbB family), or proteins resulting from chromosomal translocations. The cancer antigens also encompass antigens encoded by pathogenic organisms (bacteria, viruses, parasites, fungi, viroids or prions) that are capable of inducing a malignant condition in a subject (especially chronically infected subject) such as RNA and DNA tumor viruses (e.g.: HPV, HCV, EBV, etc.) and bacteria (e.g.: Helicobacter pilori). Cancer antigens can also be neoantigens, which are specific for a patient's tumor, and used for personalized medicine (EP2018/066668).

Some non-limiting examples of cancer antigens include, without limitation, MART-1/Melan-A, gp100, Dipeptidyl peptidase IV (DPPIV), cyclophilin b, Colorectal associated antigen, Carcinoembryonic Antigen (CEA), Prostate Specific Antigen (PSA), prostate-specific membrane antigen (PSMA), T-cell receptor/CD3-zeta chain, MAGE-family of tumor antigens, GAGE-family of tumor antigens, BAGE, RAGE, LAGE-1, NAG, GnT-V, MUM-1, CDK4, tyrosinase, p53, MUC family (e.g. MUC1, MUC16, etc.; see e.g. U.S. Pat. No. 6,054,438; WO98/04727; or WO98/37095), HER2/neu, p21ras, alpha-fetoprotein, E-cadherin, catenin family, and viral antigens such as the HPV-16 and HPV-18 E6 and E7 antigens.

Other antigens suitable for use in this invention are marker antigens (beta-galactosidase, luciferase, green fluorescent proteins, etc.).

### Suicide Polypeptide

In one embodiment, the recombinant (wild-type or variant) chimeric poxvirus of the invention may encode at least a suicide polypeptide. The term “suicide polypeptide” refers to a polypeptide able to convert a precursor of a drug, also named “prodrug”, into a cytotoxic compound. Examples of suicide polypeptides suitable for use herein and corresponding prodrugs are disclosed in the following table:

| Suicide polypeptide | Prodrug |
|---|---|
| Thymidine Kinase | Ganciclovir; |
| | Ganciclovir elaidic acid ester; |
| | Penciclovir; |
| | Acyclovir; |
| | Valacyclovir; |
| | I-5-(2-bromovinyl)-2′-deoxyuridine; |
| | Zidovudine; |
| | 2′-Exo-methanocarbathymidine |
| Cytosine deaminase | 5-Fluorocytosine |
| Purine nucleoside phosphorylase | 6-Methylpurine deoxyriboside; |
| | Fludarabine |
| Uracil phosphoribosyl transferase | 5-Fluorocytosine; |
| | 5-Fluorouracil |
| Thymidylate kinase | Azidothymidine |

In this embodiment, for at least one tumor, the oncolytic power of said recombinant chimeric poxvirus encoding a suicide polypeptide is higher than the oncolytic power measured in the same conditions and at the same time post-infection of at least one of the oncolytic recombinant parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) encoding a suicide polypeptide. In a preferred embodiment, for at least one tumor, the oncolytic power measured in the same conditions and at the same time post-infection of the recombinant parental Vaccinia virus strain Copenhagen (COP) encoding a suicide gene or the recombinant parental Rabbitpox virus strain Utrecht (RPX) encoding a suicide gene. More preferably, for at least one tumor, the oncolytic power of said recombinant chimeric poxvirus encoding a suicide gene is higher than the oncolytic power measured in the same conditions and at the same time post-infection of at least two of the recombinant oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) encoding a suicide gene. For instance, for at least one tumor, the oncolytic power of said recombinant chimeric poxvirus encoding a suicide gene is higher than the oncolytic powers measured in the same conditions and at the same time post-infection of the recombinant parental Vaccinia virus strain Copenhagen (COP) encoding a suicide gene and the recombinant parental Rabbitpox virus strain Utrecht (RPX) encoding a suicide gene. In a more preferred embodiment, for at least one tumor, the oncolytic power of said recombinant chimeric poxvirus encoding a suicide gene is higher than the oncolytic power measured in the same conditions and at the same time post-infection of at least three, more preferably higher than at least four, and even more preferably higher than each of the five oncolytic recombinant parental poxvirus strains Rabbitpox virus strain Utrecht (RPX), Cowpox virus strain Brighton (CPX), Vaccinia virus strain Copenhagen (COP), Vaccinia virus strain Wyeth (WY) and Vaccinia virus strain Western Reserve (WR) encoding a suicide gene.

Alternatively, or in combination, the invention provides a recombinant chimeric poxvirus encoding a suicide polypeptide, wherein for at least one healthy cell (preferably primary cell), the viral replication in the healthy cell of said chimeric recombinant poxvirus encoding a suicide polypeptide is lower than at least one of the five oncolytic recombinant parental poxvirus strains RPX, CPX, COP, WY and WR encoding a suicide polypeptide, preferably lower than the recombinant parental COP encoding a suicide gene or the recombinant parental RPX encoding a suicide gene, more preferably lower than at least two of the recombinant oncolytic parental poxvirus strains RPX, CPX, COP, WY and WR encoding a suicide gene (e.g.: the recombinant parental COP encoding a suicide gene and the recombinant parental RPX encoding a suicide gene), more preferably lower than at least three, more preferably lower than at least four, and even more preferably lower than each of the five oncolytic recombinant parental poxvirus strains RPX, CPX, COP, WY and WR encoding a suicide gene.

Alternatively, or in combination, the invention provides a recombinant chimeric poxvirus encoding a suicide polypeptide, wherein for at least one organ, the therapeutic index of said recombinant chimeric poxvirus encoding a suicide polypeptide is higher than the therapeutic index measured in the same conditions and at the same time post-infection of at least one of the oncolytic recombinant parental poxvirus strains RPX, CPX, COP, WY and WR encoding a suicide polypeptide, preferably higher than the therapeutic index measured in the same conditions and at the same time post-infection of the recombinant parental COP encoding a suicide gene or the recombinant parental RPX encoding a suicide gene, more preferably higher than the therapeutic index measured in the same conditions and at the same time post-infection of at least two of the recombinant oncolytic parental poxvirus strains RPX, CPX, COP, WY and WR encoding a suicide gene (e.g.: the recombinant parental COP encoding a suicide gene and the recombinant parental RPX encoding a suicide gene), more preferably higher than the therapeutic index measured in the same conditions and at the same time post-infection of at least three, more preferably of at least four, and even more preferably of each of the five oncolytic recombinant parental poxvirus strains RPX, CPX, COP, WY and WR encoding a suicide gene.

Alternatively, or in combination, the invention provides a recombinant chimeric poxvirus encoding a suicide polypeptide, wherein for at least one producer cell (preferably a tumor cell), the EEV-SC of said recombinant chimeric poxvirus encoding a suicide polypeptide is higher than the EEV-SC measured in the same conditions and at the same time post-infection of at least one of the recombinant parental poxvirus strains RPX, CPX, COP, WY, WR and MVA encoding a suicide polypeptide, preferably higher than the EEV-SC measured in the same conditions and at the same time post-infection of at least two of the recombinant parental poxvirus strains RPX, CPX, COP, WY, WR and MVA encoding a suicide gene (e.g.: the recombinant parental COP encoding a suicide gene and the recombinant parental RPX encoding a suicide gene), even more preferably higher than the EEV-SC measured in the same conditions and at the same time post-infection of at least three, preferably of least four, more preferably of at least five, even more preferably of each of the six recombinant parental poxvirus strains RPX, CPX, COP, WY, WR and MVA encoding a suicide gene.

Alternatively, or in combination, the invention provides a recombinant chimeric poxvirus encoding a suicide polypeptide wherein, for at least one tumor cell, the spreading capacity of said recombinant chimeric poxvirus encoding a suicide polypeptide is higher than the spreading capacity measured in the same conditions and at the same time post-infection of at least one of the recombinant parental poxvirus strains RPX, CPX, COP, WY, WR and MVA encoding a suicide polypeptide, preferably higher than the spreading capacity measured in the same conditions and at the same time post-infection of the recombinant parental COP encoding a suicide gene or the recombinant parental RPX encoding a suicide gene, more preferably higher than the spreading capacity measured in the same conditions and at the same time post-infection of at least two of the recombinant parental poxvirus strains RPX, CPX, COP, WY, WR and MVA encoding a suicide gene (e.g.: the recombinant parental COP encoding a suicide gene and the recombinant parental RPX encoding a suicide gene), even more preferably higher than the spreading capacity measured in the same conditions and at the same time post-infection of at least three, preferably of at least four, more preferably of at least five, even more preferably of each of the six recombinant parental poxvirus strains RPX, CPX, COP, WY, WR and MVA encoding a suicide gene.

Alternatively, or in combination, the invention provides a recombinant chimeric poxvirus encoding a suicide polypeptide wherein, for at least one poxvirus-specific antibody and tumor, the neutralization rate of said recombinant chimeric poxvirus encoding a suicide polypeptide is lower than the neutralization rate measured in the same conditions and at the same time post-infection of at least one of the recombinant parental poxvirus strains RPX, CPX, COP, WY, WR and MVA encoding a suicide polypeptide, preferably lower than the neutralization rate measured in the same conditions and at the same time post-infection of the recombinant parental COP encoding a suicide gene or the recombinant parental RPX encoding a suicide gene, more preferably lower than the neutralization rate measured in the same conditions and at the same time post-infection of at least two of the recombinant parental poxvirus strains RPX, CPX, COP, WY, WR and MVA encoding a suicide gene (e.g.: the recombinant parental COP encoding a suicide gene and the recombinant parental RPX encoding a suicide gene), even more preferably lower than the neutralization rate measured in the same conditions and at the same time post-infection of at least three, preferably of at least four, more preferably of at least five, and even more preferably of each of the six recombinant parental poxvirus strains RPX, CPX, COP, WY, WR and MVA encoding a suicide gene.

Alternatively, or in combination, the invention provides a recombinant chimeric poxvirus encoding a suicide polypeptide wherein the complement-mediated virus neutralization rate of said recombinant chimeric poxvirus encoding a suicide polypeptide is lower than the complement-mediated virus neutralization rate measured in the same conditions of at least one of the recombinant parental poxvirus strains RPX, CPX, COP, WY, WR and MVA all encoding a suicide polypeptide, preferably lower than the complement-mediated virus neutralization rate measured in the same conditions of the recombinant parental COP encoding a suicide gene, more preferably lower than the complement-mediated virus neutralization rate measured in the same conditions of at least two of the recombinant parental poxvirus strains RPX, CPX, COP, WY, WR and MVA all encoding a suicide gene (e.g.: the recombinant parental COP encoding a suicide gene and the recombinant parental RPX encoding a suicide gene), even more preferably lower than the complement-mediated virus neutralization rate measured in the same conditions of at least three, preferably of at least four, more preferably of at least five, and even more preferably of each of the six recombinant parental poxvirus strains RPX, CPX, COP, WY, WR and MVA all encoding a suicide gene.

Alternatively, or in combination, the invention provides a recombinant chimeric poxvirus encoding a suicide polypeptide, wherein for at least one producer cell (preferably a tumor cell), the syncytia formation capacity of said recombinant chimeric poxvirus encoding a suicide polypeptide is higher than the syncytia formation capacity measured in the same conditions and at the same time post-infection of at least one of the recombinant parental poxvirus strains RPX, CPX, COP, WY, WR and MVA all encoding a suicide polypeptide, preferably higher than the syncytia formation capacity measured in the same conditions and at the same time post-infection of at least two of the recombinant parental poxvirus strains RPX, CPX, COP, WY, WR and MVA all encoding a suicide gene (e.g.: the recombinant parental COP encoding a suicide gene and the recombinant parental RPX encoding a suicide gene), even more preferably higher than the syncytia formation capacity measured in the same conditions and at the same time post-infection of at least three, preferably of least four, more preferably of at least five, even more preferably of each of the six recombinant parental poxvirus strains RPX, CPX, COP, WY, WR and MVA all encoding a suicide gene.

Preferably, the recombinant chimeric poxvirus of the invention carries in its genome a gene encoding a suicide polypeptide having at least a cytosine deaminase (CDase) activity. Alternatively, or in combination, the recombinant chimeric poxvirus of the invention carries in its viral genome a gene encoding a suicide polypeptide having uracil phosphoribosyl transferase (UPRTase) activity. CDase converts 5-fluorocytosine (5-FC), thereby forming cytotoxic 5-fluorouracil (5-FU), which is then converted into the even more toxic 5-fluoro-UMP (5-FUMP). Preferably, the recombinant chimeric poxvirus of the invention encodes a suicide polypeptide engineered by fusion of two enzymatic domains, one having the CDase activity and the second having the UPRTase activity. Exemplary polypeptides include without limitation fusion polypeptides 86mou::upp, FCY1::FUR1 and FCY1::FUR1 [Delta] 10⁵ (FCU1) and FCU1-8 described in WO96/16183, EP998568 and WO2005/07857. Of particular interest is the FCU1 suicide gene (or FCY1::FUR1 [Delta] 10⁵ fusion) encoding a polypeptide comprising the amino acid sequence represented in the sequence identifier SEQ ID NO: 1 of WO2009/065546.

### Permease

According to another embodiment, the recombinant (wild-type or variant) chimeric poxvirus of the invention may comprise a nucleic acid of interest encoding a permease.

As used herein, the term “permease” refers to trans-membranous proteins involved in the translocation of nucleoside and nucleobases. Examples of permeases which are involved in the translocation of nucleosides, nucleoside analogues and nucleobases are hCNT1, hCNT2, hCNT3, hENT1 and hENT2. hCNT1, hCNT2 and hCNT3 proteins translocate nucleosides in a Na+coupled manner with high affinity and some substrate selectivity, being hCNT1 and hCNT2 pyrimidine- and purine-preferring, respectively, and hCNT3 abroad selectivity transporter. hENT1 and hENT2 are unequivocally implicated in the translocation of nucleosides and nucleobases (Pastor-Anglada et al, 2015, Front. Pharmacol., 6 (13): 1-14).

### Other Nucleic Acids of Interest:

Other nucleic acids of interest include, but are not limited to:
Nucleoside pool modulators (e.g.: cytidine deaminase, like yeast cytidine deaminase (CDD1) or human cytidine deaminase (hCD) (see EP3562946))
Agents targeting metabolic immune modulators (e.g.: adenosine deaminase like human adenosine deaminase (huADA1 or huADA2) (see EP17306012.0))
Apoptotic genes, including pro-apoptotic genes, inhibitors of pro-apoptotic genes, anti-apoptotic genes and inhibitors of anti-apoptotic genes,
Nucleic acid coding for endonuclease, like restriction enzymes, CRISPR/Cas9
RNA, including but not limited to target-specific miRNA, shRNA, siRNA.

The nucleic acid(s) of interest sequences may be easily obtained by cloning, by PCR or by chemical synthesis using conventional techniques. They may be native nucleic acid(s) sequences (e.g.: cDNA) or sequences derived from the latter by mutation, deletion, substitution and/or addition of one or more nucleotides. Moreover, their sequences are described in the literature which can be consulted by persons skilled in the art. The nucleic acid(s) sequences can be inserted at any location of the viral genome, with a specific preference for a non-essential locus (e.g.: within J2R, 14L or F4L loci).

### Expression of the Heterologous Nucleic Acid(s) of Interest

The heterologous nucleic acid(s) of interest can be independently optimized for providing high level expression in a particular host cell or subject. It has been indeed observed that, the codon usage patterns of organisms are highly non-random, and the use of codons may be markedly different between different hosts. As such nucleic acid(s) might be from bacterial or lower eukaryote origin, they may have an inappropriate codon usage pattern for efficient expression in higher eukaryotic cells (e.g.: human). Typically, codon optimization is performed by replacing one or more “native” (e.g.: bacterial or yeast) codon, corresponding to a codon infrequently used in the host organism of interest, by one or more codon encoding the same amino acid which is more frequently used. It is not necessary to replace all native codons corresponding to infrequently used codons since increased expression can be achieved even with partial replacement.

Further to optimization of the codon usage, expression in the host cell or subject can further be improved through additional modifications of the nucleic acid sequence. For example, it may be advantageous to prevent clustering of rare, non-optimal codons being present in concentrated areas and/or to suppress or modify “negative” sequence elements which are expected to negatively influence expression levels. Such negative sequence elements include without limitation the regions having very high (>80%) or very low (<30%) GC content; AT-rich or GC-rich sequence stretches; unstable direct or inverted repeat sequences; and/or internal cryptic regulatory elements such as internal TATA-boxes, chi-sites, ribosome entry sites, and/or splicing donor/acceptor sites.

In a specific embodiment of the present invention, the recombinant chimeric poxvirus comprises the elements necessary for the expression of the heterologous nucleic acid(s) of interest in a host cell subject. Specifically, such nucleic acid(s) is/are operably linked to suitable regulatory elements that allow, contribute or modulate expression in a given host cell or subject, including replication, duplication, transcription, splicing, translation, stability and/or transport of the nucleic acid(s) or its derivative (i.e.: mRNA). As used herein, “operably linked” means that the elements being linked are arranged so that they function in concert for their intended purposes. For example, a promoter is operably linked to a nucleic acid molecule if the promoter effects transcription from the transcription initiation to the terminator of said nucleic acid molecule.

It will be appreciated by those skilled in the art that the choice of the regulatory sequences can depend on such factors as the nucleic acid molecule itself, the virus into which it is inserted, the host cell or subject, the level of expression desired, etc. The promoter is of special importance. In the context of the invention, it can be constitutive directing expression of the encoded product (e.g.: polypeptide(s) encoded by a cytidine gene) in many types of host cells or specific to certain host cells (e.g.: liver-specific regulatory sequences) or regulated in response to specific events or exogenous factors (e.g.: by temperature, nutrient additive, hormone, etc.) or according to the phase of a viral cycle (e.g.: late or early). One may also use promoters that are repressed during the production step in response to specific events or exogenous factors, in order to optimize the chimeric poxvirus production and circumvent potential toxicity of the expressed polypeptide(s).

Vaccinia virus promoters are particularly adapted in the context of the invention. Representative examples include without limitation the vaccinia p7.5K, pH5R, p11k7.5 (Erbs et al., 2008, Cancer Gene Ther. 15 (1): 18-28), pSE, pTK, p28, p11, pB2R, pF17R, pA14L, pSE/L, pA35R, pK1L, pPr13.5 (WO2014/063832), pB8R, pF11L, pA44L, pC11R (WO2011/128704), as well as synthetic promoters such as those described in Chakrabarti et al. (1997, Biotechniques, 23:1094-7; Hammond et al., 1997, J. Virol. Methods, 66:135-8; and Kumar and Boyle, 1990, Virology, 179:151-8) as well as early/late chimeric promoters (e.g.: U.S. Pat. Nos. 8,394,385; 8,772,023). Cowpox promoters are also suitable as well (e.g.: the ATI promoter).

In a preferred embodiment, the IL-12 is inserted in the 14L locus of the recombinant chimeric poxvirus of the invention and placed under the control of the vaccinia pH5R promoter.

Those skilled in the art will appreciate that the regulatory elements controlling the nucleic acid expression may further comprise additional elements for proper initiation, regulation and/or termination of transcription (e.g. a transcription termination sequences), mRNA transport (e.g. nuclear localization signal sequences), processing (e.g. splicing signals), and stability (e.g. introns and non-coding 5′ and 3′ sequences), translation (e.g. an initiator Met, tripartite leader sequences, IRES ribosome binding sites, signal peptides, etc.), targeting sequences, transport sequences, secretion signal, and sequences involved in replication or integration. Said sequences have been reported in the literature and can be readily obtained by those skilled in the art.

### Process for Producing a Chimeric Poxvirus

The invention also relates to a process for producing a (wild-type, variant, recombinant or recombinant variant) chimeric poxvirus of the invention, said process comprising:
(i) infecting a producer cell with the chimeric poxvirus of the invention
(ii) culturing said infected producer cell under conditions which are appropriate for enabling said chimeric poxvirus particles to be produced, and
(iii) recovering said chimeric poxvirus particles from the producer cell culture.

The chimeric poxvirus of the present invention is produced into suitable producer cells using conventional techniques including culturing the transfected or infected host cells under suitable conditions so-as to allow the production of infectious poxviral particles and recovering the produced infectious viral particles from the culture of said cells and optionally purifying said recovered infectious viral particles. Suitable host cells for production of the chimeric poxvirus include without limitation human cells such as HeLa (ATCC), 293 cells (Graham et al., 1997, J. Gen. Virol. 36:59-72), HER96, PER-C6 (Fallaux et al., 1998, Human Gene Ther. 9:1909-17), Monkey cells such as Vero (ATCC CCL-081), CV-1 (ATCC CCL-70) and BSC1 (ATCC CCL-26) cell lines, avian cells such as those described in WO2005/042728, WO2006/108846, WO2008/129058, WO2010/130756, WO2012/001075, etc.), hamster cell lines such as BHK-21 (ATCC CCL-10), primary chicken embryo fibroblasts (CEF) prepared from chicken embryos obtained from fertilized eggs, EB66®, HEK 293, BHK21, or MRC5 cells. Host cells are preferably cultivated in a medium free of animal- or human-derived products, using a chemically defined medium with no product of animal or human origin. Culturing is carried out at a temperature, pH and oxygen content appropriate for the producer cell. Such culturing conditions are within the expertise of one of ordinary skill in the art. If growth factors are present, they are preferably recombinantly produced and not purified from animal material. Suitable animal-free medium media are commercially available, for example VP-SFM medium (Invitrogen) for culturing CEF producer cells. Producer cells are preferably cultivated at a temperature comprised between +30° C. and +38° C. (more preferably at about +37° C.) for between 1 and 8 days (preferably for 1 to 5 days for CEF and 2 to 7 days for immortalized cells) before infection. If needed, several passages of 1 to 8 days may be made in order to increase the total number of cells.

Producer cells are infected by the chimeric poxvirus with an appropriate multiplicity of infection (MOI), which can be as low as 0.001 (more preferably between 0.05 and 5) to permit productive infection.

In step ii), infected producer cells are then cultured under appropriate conditions well known to those skilled in the art until progeny viral vector is produced. Culture of infected producer cells is also preferably performed in a chemically defined medium (which may be the same as or different from the medium used for culture of producer cells and/or for infection step) free of animal- or human-derived products at a temperature between +30° C. and +37° C., for 1 to 5 days.

In step iii), the viral particles may be collected from the culture supernatant and/or the producer cells. Recovery from producer cells (and optionally also from culture supernatant), may require a step allowing the disruption of the producer cell membrane to allow the liberation of the virus from producer cells. The disruption of the producer cell membrane can be induced by various techniques well known to those skilled in the art, including but not limited to, freeze/thaw, hypotonic lysis, sonication, microfluidization, or high-speed homogenization.

The recovered chimeric poxvirus can be at least partially purified before being used according to the present invention. Various purification steps can be envisaged, including clarification, enzymatic treatment (e.g.: endonuclease such as benzonase, protease), ultracentrifugation (e.g.: sucrose gradient or cesium chloride gradient), chromatographic and filtration steps. Appropriate methods are described in the art (e.g.: WO2007/147528; WO2008/138533, WO2009/100521, WO2010/130753, WO2013/022764).

### Composition

The invention also relates to a composition (preferentially a pharmaceutical composition) that comprises a therapeutically effective amount of the (wild-type, variant, recombinant or recombinant variant) chimeric poxvirus of the present invention. Preferably, the composition further comprises a pharmaceutically acceptable vehicle. Such a composition may be administered once or several times (e.g.: 2, 3, 4, 5, 6, 7 or 8 times etc.) and via the same or different routes.

A “therapeutically effective amount” corresponds to the amount of each of the active agents comprised in the composition of the invention that is sufficient for producing one or more beneficial results. Such a therapeutically effective amount may vary as a function of various parameters, e.g.: the mode of administration; the disease state; the age and weight of the subject; the ability of the subject to respond to the treatment; kind of concurrent treatment; the frequency of treatment; and/or the need for therapy. For “therapeutic” use, the composition of the invention is administered to a subject diagnosed as having a pathological condition (e.g.: a proliferative disease such as cancer) with the goal of treating the disease, eventually in association with one or more conventional therapeutic modalities. In particular, a therapeutically effective amount could be that amount necessary to cause an observable improvement of the clinical status over the baseline status or over the expected status if not treated, as described hereinafter. An improvement of the clinical status can be easily assessed by any relevant clinical measurement typically used by physicians and skilled healthcare staff. For example, techniques routinely used in laboratories (e.g.: flow cytometry, histology, imaging techniques, etc.) may be used to perform tumor surveillance. A therapeutically effective amount could also be the amount necessary to cause the development of an effective non-specific (innate) and/or specific anti-tumor response. Typically, development of an immune response, in particular a T cell response, can be evaluated in vitro, in suitable animal models or using biological samples collected from the subject. One may also use various available antibodies so-as to identify different immune cell populations involved in anti-tumor response that are present in the treated subjects, such as cytotoxic T cells, activated cytotoxic T cells, natural killer cells and activated natural killer cells.

The appropriate dosage of the (wild-type, variant, recombinant or recombinant variant) chimeric poxvirus can be adapted as a function of various parameters and may be routinely determined by a practitioner in the light of the relevant circumstances. Suitably, individual doses for the (wild-type, variant, recombinant or recombinant variant) chimeric poxvirus may vary within a range extending from approximately 103 to approximately 10¹² vp (viral particles), iu (infectious unit) or PFU (plaque-forming units) depending on the virus and the quantitative technique used. For illustrative purposes, a suitable dose of (wild-type, variant, recombinant or recombinant variant) chimeric poxvirus or recombinant chimeric poxvirus for human use is comprised between approximately 10⁴ to approximately 10¹¹ PFU, preferably between approximately 10⁵ PFU to approximately 10¹⁰ PFU; doses of approximately 10⁶ PFU to approximately 5×10⁹ PFU being particularly preferred (e.g. dose of 10⁶, 2×10⁶, 3×10⁶, 4×10⁶, 5×10⁶, 6×10⁶, 7×10⁶, 8×10⁶, 9×10⁶, 10⁷, 2×10⁷, 3×10⁷, 4×10⁷, 5×10⁷, 6×10⁷, 7×10⁷, 8×10⁷, 9×10⁷, 10⁸, 2×10⁸, 3×10⁸, 4×10⁸, 5×10⁸, 6×10⁸, 7×10⁸, 8×10⁸, 9×10⁸, 109, 2×10⁹, 3×10⁹, 4×10⁹ or 5×10⁹ PFU). The quantity of virus present in a sample can be determined by routine titration techniques, e.g.: by counting the number of plaques following infection of permissive cells (e.g.: BHK-21 or CEF), immunostaining (e.g.: using anti-virus antibodies; Caroll et al., 1997, Virology 238:198-211), by measuring the A260 absorbance (vp titers), or still by quantitative immunofluorescence (iu titers).

The term “pharmaceutically acceptable vehicle” is intended to include any and all carriers, solvents, diluents, excipients, adjuvants, dispersion media, coatings, antibacterial and antifungal agents, absorption agents and the like compatible with administration in mammals and in particular human subjects.

The (wild-type, variant, recombinant or recombinant variant) chimeric poxvirus of the invention can independently be placed in a solvent or diluent appropriate for human or animal use. The solvent or diluent is preferably isotonic, hypotonic or weakly hypertonic and has a relatively low ionic strength. Representative examples include sterile water, physiological saline (e.g.: sodium chloride), Ringer's solution, glucose, trehalose or saccharose solutions, Hank's solution, and other aqueous physiologically balanced salt solutions (see for example the most current edition of Remington: The Science and Practice of Pharmacy, A. Gennaro, Lippincott, Williams&Wilkins).

In one embodiment, (wild-type, variant, recombinant or recombinant variant) chimeric poxviruses are suitably buffered for human use. Suitable buffers include without limitation phosphate buffer (e.g.: PBS), bicarbonate buffer and/or Tris buffer capable of maintaining a physiological or slightly basic pH (e.g. from approximately pH 7 to approximately pH 9).

The composition of the invention may also contain other pharmaceutically acceptable excipients for providing desirable pharmaceutical or pharmacodynamic properties, including for example osmolarity, viscosity, clarity, colour, sterility, stability, rate of dissolution of the formulation, modifying or maintaining release or absorption into a human or animal subject, promoting transport across the blood barrier or penetration in a particular organ.

In one embodiment, the composition of the invention can also comprise one or more adjuvants capable of stimulating immunity (especially a T cell-mediated immunity) or facilitating infection of tumor cells upon administration, e.g. through toll-like receptors (TLR) such as TLR-7, TLR-8 and TLR-9, including without limitation alum, mineral oil emulsion such as, Freunds complete and incomplete (IFA), lipopolysaccharide or a derivative thereof (Ribi et al., 1986, Plenum Publ. Corp., 407-419), saponins such as QS21 (Sumino et al., 1998, J.Virol. 72:4931; WO98/56415), imidazoquinoline compounds such as Imiquimod (Suader, 2000, J. Am Acad Dermatol. 43: S6), S-27609 (Smorlesi, 2005, Gene Ther. 12:1324) and related compounds such as those described in WO2007/147529, polysaccharides such as Adjuvax and squalenes, oil in water emulsions such as MF59, double-stranded RNA analogs such as poly (I: C), single stranded cytosine phosphate guanosine oligodeoxynucleotides (CpG) (Chu et al., 1997, J. Exp. Med., 186:1623; Tritel et al., 2003, J. Immunol., 171:2358) and cationic peptides such as IC-31 (Kritsch et al., 2005, J. Chromatogr. Anal. Technol. Biomed. Life Sci., 822:263-70).

In one embodiment, the composition of the invention may be formulated with the goal of improving its stability, in particular under the conditions of manufacture and long-term storage (i.e.: for at least 6 months, with a preference for at least two years) at freezing (e.g.:−70° C., −20° C.), refrigerated (e.g.: 4° C.) or ambient temperatures. Various virus formulations are available in the art either in frozen, liquid form or lyophilized form (e.g.: WO98/02522, WO01/66137, WO03/053463, WO2007/056847 and WO2008/114021, etc.). Solid (e.g.: dry powdered or lyophilized) compositions can be obtained by a process involving vacuum drying and freeze-drying (see e.g.: WO2014/053571). For illustrative purposes, buffered formulations including NaCl and/or sugar are particularly adapted to the preservation of viruses (e.g. S01 buffer: 342.3 g/L saccharose, 10 mM Tris, 1 mM MgCl2, 150 mM NaCl, 54 mg/L, Tween 80; ARME buffer: 20 mM Tris, 25 mM NaCl, 2.5% Glycerol (w/v), pH 8.0; S520 buffer: 100 g/L saccharose, 30 mM Tris, pH 7.6; S08 buffer: 10 mM Tris, 50 mM NaCl, 50 g/L saccharose, 10 mM Sodium glutamate, pH 8.0).

The composition is preferably formulated in a way adapted to the mode of administration to ensure proper distribution and release in vivo. For example, gastro-resistant capsules and granules are particularly appropriate for oral administration, suppositories for rectal or vaginal administration, eventually in combination with absorption enhancers useful to increase the pore size of the mucosal membranes. Such absorption enhancers are typically substances having structural similarities to the phospholipid domains of the mucosal membranes (such as sodium deoxycholate, sodium glycocholate, dimethyl-beta-cyclodextrin, lauryl-1-lysophosphatidylcholine). Another example relates to the use of cell carriers (e.g.: mesenchymal stem cells, neural stem cells) as a vehicle for virus delivery. Another and particularly appropriate example is a formulation adapted to the administration through microneedle means (e.g. transcutaneous or intradermal patches). Such a formulation may comprise resuspension of the immunotherapeutic product in endotoxin-free phosphate-buffered saline (PBS). It can also be formulated in liposomes. Biodegradable, biocompatible polymers can be used, such as ethylene vinyl acetate, polyanhydrides, polyglycolic acid, collagen, polyorthoester, and polylactic acid. Many methods for the preparation of such formulations are described by e.g.: J. R. Robinson in “Sustained and Controlled Release Drug Delivery Systems”, ed., Marcel Dekker, Inc., New York, 1978.

### Administration

The (wild-type, variant, recombinant or recombinant variant) chimeric poxvirus or the composition of the invention may be administered in a single dose or multiple doses. If multiples doses are contemplated, administrations may be performed by the same or different routes and may take place at the same site or at alternative sites. Intervals between each administration can vary from approximately 1 day to approximately 8 weeks (e.g.: 24 h, 48h, 72h, weekly, every two or three weeks, monthly, etc.), advantageously from approximately 2 days to approximately 6 weeks, preferably from approximately 3 days to approximately 4 weeks and even more preferably from approximately 1 week to approximately 3 weeks (e.g.: every two weeks for example). Intervals can also be irregular. It is also possible to proceed via sequential cycles of administrations that are repeated after a rest period (e.g.: cycles of 3 to 6 weekly administrations followed by a rest period of 3 to 6 weeks). The dose can vary for each administration within the range described above.

Any of the conventional administration routes are applicable in the context of the invention including parenteral, topical or mucosal routes. Parenteral routes are intended for administration as an injection or infusion and encompass systemic as well as local routes. Common parenteral injection types are intravenous (into a vein), intra-arterial (into an artery), intradermal (into the dermis), subcutaneous (under the skin), intramuscular (into a muscle) and intratumoral (into a tumor or at its proximity). Infusions typically are given by intravenous route. Topical administration can be performed using transdermal means (e.g.: patch and the like). Mucosal administrations include without limitation oral/alimentary, intranasal, intratracheal, intrapulmonary, intravaginal or intra-rectal route. In the case of intranasal, intrapulmonary and intratracheal routes, it is advantageous for administration to take place by means of an aerosol or by means of instillation. Preferred routes of administration for the chimeric vaccinia virus of the invention include intravenous and intratumoral routes.

Administrations may use conventional syringes and needles (e.g.: Quadrafuse injection needles) or any compound or device available in the art capable of facilitating or improving delivery of the active agent(s) in the subject. Transdermal systems are also appropriate, e.g.: using solid, hollow, coated or dissolvable microneedles (e.g.: Van der Maaden et al., 2012, J. Control release 161:645-55) and preferred are silicon and sucrose microneedle patches (see, e.g., Carrey et al., 2014, Sci Rep 4:6154 doi 10.1038; and Carrey et al., 2011, PloS ONE, 6 (7) e22442).

A particularly preferred composition comprises 10⁶ PFU to 5×10⁹ PFU of a (wild-type, variant, recombinant or recombinant variant) chimeric poxvirus according to the invention formulated for intravenous or intratumoral administration. Another preferred composition comprises 10⁶ PFU to 5×10⁹ PFU of a variant and/or recombinant variant chimeric poxvirus according to the invention defective in the J2R locus formulated for intravenous or intratumoral administration. Another particularly preferred composition comprises 10⁶ PFU to 5×10⁹ PFU of a variant and/or recombinant variant chimeric poxvirus according to the invention defective in the J2R locus and 14L and/or F4L locus formulated for intravenous or intratumoral administration. Another particularly preferred composition comprises 10⁶ PFU to 5×10⁹ PFU of a recombinant variant chimeric poxvirus according to the invention having the IL-12 inserted in place of the 14L locus and placed under the pH5R promoter such as chimeric poxvirus TK-/IL-12 described herein formulated for intravenous or intratumoral administration.

The (wild-type, variant, recombinant or recombinant variant) chimeric poxvirus of the invention can be associated with one or more substances effective in anticancer therapy. Among pharmaceutical substances effective in anticancer therapy which may be used in association or in combination with the chimeric poxvirus of the invention, there may be mentioned more specifically:
alkylating agents such as e.g.: mitomycin C, cyclophosphamide, busulfan, ifosfamide, melphalan, hexamethylmelamine, thiotepa, chlorambucil, or dacarbazine;
antimetabolites such as, e.g.: gemcitabine, capecitabine, 5-fluorouracil, cytarabine, 2-fluorodeoxy cytidine, methotrexate, idatrexate, tomudex or trimetrexate;
topoisomerase II inhibitors such as, e.g.: doxorubicin, epirubicin, etoposide, teniposide or mitoxantrone;
topoisomerase I inhibitors such as, e.g.: irinotecan (CPT-11), 7-ethyl-10-hydroxy-camptothecin (SN-38) or topotecan;
antimitotic drugs such as, e.g.: paclitaxel, docetaxel, vinblastine, vincristine or vinorelbine;
platinum derivatives such as, e.g.: cisplatin, oxaliplatin, spiroplatinum or carboplatinum;
inhibitors of tyrosine kinase receptors such as sunitinib (Pfizer) and sorafenib (Bayer); and
anti-neoplastic antibodies
cell carriers such as neural stem cells or mesenchymal stem cells sodium Iodide Symporter-Radioiodine Gene Therapy

The (wild-type, variant, recombinant or recombinant variant) chimeric poxvirus of the invention may also be used in association with one or more other agents including but not limited to immunomodulatory agents such as, e.g. alpha, beta or gamma interferon, interleukin (in particular IL-2, IL-6, IL-10 or IL-12) or tumor necrosis factor; CAR-T cells; agents that affect the regulation of cell surface receptors such as, e.g. inhibitors of Epidermal Growth Factor Receptor (in particular cetuximab, panitumumab, zalutumumab, nimotuzumab, matuzumab, gefitinib, erlotinib or lapatinib) or inhibitors of Human Epidermal Growth Factor Receptor-2 (in particular trastuzumab); agents that affect angiogenesis such as, e.g. inhibitor of Vascular Endothelial Growth Factor (in particular bevacizumab or ranibizumab); Immune Checkpoint Inhibitor (ICI), also designated as Immune Checkpoint Modulator (ICM), e.g. anti-PD1, anti-PD-L1, anti-PD-L2, anti-CTLA4, anti-Lag3, anti-BTLA and anti-Tim3.

Such substances effective in anticancer therapy may be administered to the subject sequentially or concomitantly with the (wild-type, variant, recombinant or recombinant variant) chimeric poxvirus of the invention.

In the case of a product combination, the present invention also provides kits including the active agent(s) of the combination of the invention in kit form. In one embodiment, a kit includes at least a (wild-type, variant, recombinant or recombinant variant) chimeric poxvirus as described herein in one container (e.g., in a sterile glass or plastic vial), and one or more pharmaceutical substances effective in anticancer therapy in another container (e.g., in a sterile glass or plastic vial). Optionally, the kit can include a device for performing the administration of the active agents. The kit can also include a package insert including information concerning the compositions or individual component and dosage forms in the kit.

Alternatively, or in combination, the (wild-type, variant, recombinant or recombinant variant) chimeric poxvirus of the invention can also be used in association with radiotherapy.

### Methods and Uses

In another aspect, the present invention provides a (wild-type, variant, recombinant or recombinant variant) chimeric poxvirus or a composition thereof (in particular a pharmaceutical composition) for use as a drug, for treating a disease or a pathologic condition in a subject in need thereof. The present invention also relates to the use of a (wild-type, variant, recombinant or recombinant variant) chimeric poxvirus or composition thereof for the manufacture of a medicament for treating a disease or a pathologic condition in a subject in need thereof. The present invention also relates to a method of treatment comprising administering the (wild-type, variant, recombinant or recombinant variant) chimeric poxvirus or the composition thereof in an amount sufficient for treating a disease or a pathologic condition in a subject in need thereof. The present invention also relates to the use of a (wild-type, variant, recombinant or recombinant variant) chimeric poxvirus or composition thereof for treating a disease or a pathologic condition in a subject in need thereof.

A “disease” (and any form of disease such as “disorder” or “pathological condition”) is typically characterized by identifiable symptoms.

Examples of diseases that may be treated using the (wild-type, variant, recombinant or recombinant variant) chimeric poxvirus of the invention, or the composition thereof include proliferative diseases such as cancers, tumors or restenosis.

The present invention is particularly suited for treating cancers, and particularly Adrenocortical Carcinoma, Adrenal Cortex Cancer, Anal Cancer, Gastrointestinal Carcinoid Tumors (for example

Appendix Cancer and Carcinoid Tumor), Bile Duct Cancer (for example Cholangiocarcinoma), Bladder Cancer, Bone Cancer (for example Ewing Sarcoma, Malignant Fibrous Histiocytoma of Bone and Osteosarcoma), Brain Tumors (for example Astrocytomas, Embryonal Tumors, Germ Cell Tumors, Central Nervous System Atypical Teratoid/Rhabdoid Tumor, Craniopharyngioma, Ependymoma, Gliomas and Glioblastoma), Breast Cancer (for example Ductal Carcinoma In Situ), Bronchial Tumors, Carcinoma of Unknown Primary, Cardiac (Heart) Tumors, Cervical Cancer, Chordoma, Chronic Myeloproliferative Neoplasms, Colorectal Cancer (for example Colon Cancer or Rectal Cancer), Esthesioneuroblastoma, Extracranial Germ Cell Tumor, Extragonadal Germ Cell Tumor, Retinoblastoma, Gallbladder Cancer, Gastrointestinal Carcinoid Tumor, Testicular Cancer, Gestational Trophoblastic Disease, Head and Neck Cancer (for example Hypopharyngeal Cancer, pharyngeal Cancer, Laryngeal Cancer, Lip and Oral Cavity Cancer, Metastatic Squamous Neck Cancer with Occult Primary, Mouth Cancer, Nasal Cavity and Paranasal Sinus Cancer, Nasopharyngeal Cancer, Salivary Gland Cancer, Throat Cancer, Esophageal Cancer), Hepatocellular (Liver) Cancer, Histiocytosis, Langerhans Cell, Kidney cancer (for example Wilms Tumor, Renal Cell Cancer, Transitional Cell Cancer of the Renal Pelvis and Ureter), Langerhans Cell Histiocytosis, Laryngeal Cancer and Papillomatosis, Leukemia (for example Hairy Cell Leukemia, Chronic Lymphocytic Leukemia (CLL), Chronic Myelogenous Leukemia (CML), Acute Myeloid Leukemia (AML), Acute Lymphoblastic Leukemia (ALL)), Liver Cancer, Lung Cancer (Small Cell Lung Cancer and Non-Small Cell Lung Cancer), Lymphoma (for example AIDS-Related Lymphoma, Primary CNS Lymphoma, Cutaneous T-Cell Lymphoma, Hodgkin Lymphoma, Burkitt Lymphoma, Primary Lymphoma, Mycosis Fungoides, Non-Hodgkin Lymphoma, Macroglobulinemia, Waldenstrom, Primary Central Nervous System (CNS) Lymphoma, Sezary Syndrome, T-Cell Lymphoma), Intraocular Melanoma, Mesothelioma, Midline Tract Carcinoma Involving NUT Gene, Multiple Endocrine Neoplasia Syndromes, Multiple Myeloma/Plasma Cell Neoplasms Myelodysplastic Syndromes, Chronic Myeloproliferative Neoplasms, Neuroblastoma, Ovarian Cancer (for example Primary Peritoneal Cancer and Fallopian Tube Cancer), Pancreatic Cancer and Pancreatic Neuroendocrine Tumors (Islet Cell Tumors), Papillomatosis, Paraganglioma, Parathyroid Cancer, Penile Cancer, Pheochromocytoma, Pituitary Tumor, Plasma Cell Neoplasm/Multiple Myeloma, Pleuropulmonary Blastoma, Prostate Cancer, Retinoblastoma, Vascular Tumors, Skin Cancer (for example Basal Cell Carcinoma, Melanoma, Squamous Cell Carcinoma and Merkel Cell Carcinoma), Small Intestine Cancer, Soft Tissue Sarcoma (for example Gastrointestinal Stromal Tumors (GIST), AIDS-Related Cancers Kaposi Sarcoma, Kaposi Sarcoma and Rhabdomyosarcoma), Stomach (Gastric) Cancer, Testicular Cancer, Thymoma and Thymic Carcinoma, Thyroid Cancer, Urethral Cancer, Uterine Cancer, Endometrial and Uterine Sarcoma, Vaginal Cancer and Vulvar Cancer. The present invention is also useful for treatment of metastatic cancers. In a preferred embodiment, the present invention is particularly suited for treating solid cancers (including for example carcinomas and sarcomas) or hematological malignancies (including for example lymphoma, leukemia and myelomas). In a preferred embodiment, the present invention is particularly suited for treating lung cancer, renal cancer, bladder cancer, prostate cancer, breast cancer, colorectal cancer, colon cancer, hepatic cancer, hepatocarcinoma, gastric cancer, pancreatic cancer, melanoma, ovarian cancer and glioblastoma. In a more preferred embodiment, the present invention is particularly suited for treating lung cancer, colon cancer, hepatocarcinoma, pancreatic cancer, melanoma and glioblastoma. In another preferred embodiment, the present invention is particularly suited for treating cancers refractory or resistant to at least one oncolytic virus-based therapy, or to at least one oncolytic vaccinia virus-based therapy.

A particularly preferred method comprises 1 to 6 (e.g.: 3) intravenous or intratumoral administrations of the (wild-type, variant, recombinant or recombinant variant) chimeric poxvirus of the invention or the composition thereof given at weekly to monthly intervals with a specific preference for 3 bi-weekly administrations (e.g.: at approximately D1, D14 and D29) of a composition comprising 10⁶ to 5×10⁹ PFU of a (wild-type, variant, recombinant or recombinant variant) chimeric poxvirus, the latter being preferably defective in J2R locus and/or in the 14L or F4L locus.

Another particularly preferred method comprises 1 to 6 (e.g.: 3) intravenous or intratumoral administrations of the (wild-type, variant, recombinant or recombinant variant) chimeric poxvirus of the invention or the composition thereof given at weekly to monthly intervals with a specific preference for 3 bi-weekly administrations (e.g.: at approximately D1, D14 and D29) of a composition comprising 106 to 5×10⁹ PFU of a (wild-type, variant, recombinant or recombinant variant) chimeric poxvirus, the latter being preferably defective in J2R locus and/or in the 14L or F4L locus, and encoding an interleukin, more preferably encoding an IL-12.

The beneficial effects provided by the methods of the present invention can be evidenced by an observable improvement of the clinical status over the baseline status or over the expected status if not treated according to the modalities described herein. An improvement of the clinical status can be easily assessed by any relevant clinical measurement typically used by physicians and skilled healthcare staff. In the context of the invention, the therapeutic benefit can be transient (for one or a couple of months after cessation of administration) or sustained (for several months or years). As the natural course of clinical status which may vary considerably from a subject to another, it is not required that the therapeutic benefit be observed in each subject treated but in a significant number of subjects (e.g. statistically significant differences between two groups can be determined by any statistical test known in the art, such as a Tukey parametric test, the Kruskal-Wallis test the U test according to Mann and Whitney, the Student's t-test, the Wilcoxon test, etc.).

In a particular embodiment, as the methods according to the present invention are particularly appropriate for treating cancer, such methods can be correlated with one or more of the followings: inhibiting or slowing tumor growth, proliferation and metastasis, preventing or delaying tumor invasion (spread of tumor cells in neighboring tissues), reducing the tumor number; reducing the tumor size, reducing the number or extent of metastases, providing a prolonged overall survival rate (OS), increasing progression free survival (PFS), increasing the length of remission, stabilizing (i.e. not worsening) the state of disease, providing a better response to the standard treatment, improving quality of life and/or inducing an anti-tumor response (e.g. non-specific (innate) and/or specific such as a cytotoxic T cell response) in the subject treated in accordance with the present invention.

The appropriate measurements that can be used to assess a clinical benefit such as blood tests, analysis of biological fluids and biopsies as well as medical imaging techniques are evaluated routinely in medical laboratories and hospitals and a large number of kits is available commercially. They can be performed before the administration (baseline) and at various time points during treatment and after cessation of the treatment.

The present invention also relates to a method for treating a disease or a pathological condition in a subject in need thereof comprising administering the (wild-type, variant, recombinant or recombinant variant) chimeric poxvirus or the composition of the present invention or prepared according to the process described herein. In one embodiment, said disease is a proliferative disease such as cancers, tumors and restenosis. More precisely, the present invention relates to a method for inhibiting tumor cell growth in vivo comprising administering a (wild-type, variant, recombinant or recombinant variant) chimeric poxvirus or a composition thereof in a subject in need thereof so-as to inhibit the growth of a tumor. For general guidance, inhibition of tumor cell growth can be evaluated routinely, for example by radiography means. The administration(s) of the (wild-type, variant, recombinant or recombinant variant) chimeric poxvirus or the composition thereof desirably result(s) in at least a 10% decrease of the tumor mass.

All of the above cited disclosures of patents, publications and database entries are specifically incorporated herein by reference in their entirety. Other features, objects, and advantages of the invention will be apparent from the description, drawings and from the claims. The following examples are incorporated to demonstrate preferred embodiments of the invention. However, in light of the present disclosure, those skilled in the art should appreciate that changes can be made in the specific embodiments that are disclosed without departing from the spirit and scope of the invention.

### Examples

### Generation and Characterization of Poxstg19503, a Chimeric Poxvirus, and Two Variants Thereof (Single Deleted in J2R and Double Deleted in J2R and 14L)

For the sake of making the reading process easier, Table 1 is provided, comprising the description of the code references used in the Figures and Examples.

TABLE 1
Codes of the viruses (optionally variant and/or recombinant)
cited in the Description section and the Examples section.

| Code | Description |
|---|---|
| VVTG17111 | TK-deleted vaccinia virus strain |
| | Copenhagen encoding GFP::FCU1 |
| VVTG19328 | Double (TK/RR)-deleted vaccinia virus strain |
| | Copenhagen encoding mIL-12 |
| TG6002 | Double (TK/RR)-deleted vaccinia virus strain |
| | Copenhagen encoding FCU1 |
| VVTG17990 | Double (TK/RR)-deleted vaccinia virus strain |
| | Copenhagen encoding GFP |
| WRTG15467 | TK-deleted vaccinia virus strain Western |
| | Reserve encoding GFP |
| WYTG18254 | TK-deleted vaccinia virus strain Wyeth |
| | encoding hGM-CSF |
| CPXTG18831 | TK-deleted cowpox virus strain Brighton |
| | encoding GFP::FCU1 |
| RPXTG19095 | TK-deleted rabbitpox virus strain Utrecht |
| | encoding GFP |
| MVATG15938 | Vaccinia virus strain MVA encoding GFP |
| POXSTG19503 | Wild-type chimeric poxvirus |
| POXSTG19508 | TK-deleted chimeric poxvirus encoding |
| | GFP::FCU1 |
| POXSTG19730 | Double (TK/RR)-deleted chimeric poxvirus |
| | encoding GFP::FCU1 and mCherry |
| POXSTG19847 | Double (TK/RR)-deleted chimeric poxvirus |
| | encoding mIL-12 |
| POXSTG20150 | Double (TK/RR)-deleted chimeric poxvirus |

### Materials and Methods

### Cell Lines and Viruses

Human colon cancer cell line HCT116 (CCL-247™), human lung cancer cell line A549 (CCL-185™), human hepatocarcinoma cell line HepG2 (HB 8065™), human glioblastoma cancer cell line U-87 MG (HTB-14™), human pancreatic cancer cell line MIA PaCa-2 (CRL-1420™), murine melanoma cell line B16F10 (CRL-6475), murine colon carcinoma cell line CT26 (CRL-2638) and Vero cell line (CCL-81) were obtained from the American Type Culture Collection (ATCC, Rockville, MD). All cell lines were grown in recommended media supplemented with 10% fetal calf serum (FCS). Fresh human hepatocytes were purchased from Biopredic International (Rennes, France) and maintained in the recommended hepatocyte medium provided by the supplier (Biopredic International). The Phenion full-thickness skin model, a 3D tissue construct that simulates histological and physiological properties of human skin, was purchased from Henkel (Düsseldorf, Germany). This organotypic epithelial raft culture model was maintained in tissue culture medium according to the manufacturer's instructions. Primary chicken embryo fibroblasts (CEF) were used for recombination, production and titration of viral vectors. CEF cells were prepared as previously described (Foloppe et al., 2008, Gene Ther., 15, 1361-1371. doi: 10.1038/gt.2008.82) and maintained in Eagle-based Medium (MBE) supplemented with 5% FCS. Wild-type vaccinia virus Western Reserve strain (WR, ATCC VR-119), wild-type vaccinia virus Wyeth strain (WY, ATCC VR-1536™), wild-type Cowpox virus strain Brighton (CPXV, ATCC VR-302), wild-type Raccoonpox virus strain Herman (RCNV, ATCC VR-838), wild-type Rabbitpox virus strain Utrecht (RPXV, ATCC VR-1591), wild-type Bovine Papular Stomatitis virus strain Illinois 721 (BPSV, ATCC VR-801), wild-type ORF virus strain NZ2 (ORFV, ATCC VR-1548), wild-type Pseudo-cowpox virus strain TJS (PCPV, ATCC VR-634), wild-type Myxoma virus strain Lausanne (MYXV, ATCC VR-1829), wild-type Yaba-like disease virus strain Davis (YLDV, ATCC VR-937), wild-type Swinepox virus strain Kasza (SWPV, ATCC VR-363), wild-type Cotia virus strain SP AN 32 (CTV, ATCC VR-464) wild-type Squirrel Fibroma virus strain Kilham (SQFV, ATCC VR-236) and wild-type IHD-J (ATCC VR-156) were obtained from ATCC. Wild-type vaccinia virus strain Copenhagen (COP) used in the work described here comes from the Institut Merieux (Marcy l′Etoile, France). MVA expressing the eGFP gene under the control of the p11k7.5 promoter (MVATG15938) was constructed and characterized previously (Erbs et al., 2008, Cancer Gene Ther., 15, 18-28). Wild-type Fowlpox virus strain FP9 (FPV) was kindly provided by Pr Skinner. Double (TK/RR)-deleted Copenhagen strain VACVs encoding FCU1 (TG6002) or GFP (VVTG17990) were constructed and characterized previously (Foloppe et al., 2019, Mol Ther Oncolytics, 14, 1-14; Beguin et al., 2020, Mol Ther Oncolytics, 19, 57-66).

### Patient-Derived Serum Fluid

Serum was obtained from patients enrolled in phase 1 clinical trial evaluating the safety and tolerability of multiple-ascending doses of the Copenhagen strain VACV TG6002 administered intravenously in patients with advanced gastro-intestinal tumors (NCT03724071). All patients gave written informed consent according to good clinical practice guidelines. Serum samples from a patient collected one day before IV administration of TG6002 and 42 days after virus administration were used for VACV neutralization assay.

### Neutralizing Antibody Assay

A neutralizing antibody assay was used to determine the titer of VACV neutralizing antibodies (VACV-NAbs) in serum. For the serum collected before TG6002 administration, the VACV-NAb titer was below the limit of detection (<20), while this titer was 4580 for the serum collected 42 days post-infection. Two-fold serial dilutions of serum samples were incubated with the GFP expressing vaccinia virus VVTG17990 in 6-well plates for 1 hour at 37° C. The diluted serum-virus mixtures were then added to Vero Cells for VVTG17990 titration by plaque assay. After three days of incubation, Vero cell culture plates were examined using a fluorescence microscope to score the virus-infected cells (plaque). The neutralizing antibody titer was defined as the highest serum dilution preventing ≥50% plaque formation.

### Directed Evolution for Selection of Chimeric Poxvirus

Sixteen poxviruses (COP, WY, WR, MVATG15938, CPXV, RCNV, RPXV, BPSV, ORFV, PCPV, MYXV, YLDV, SWPV, CTV, SQFV, FPV) were mixed and used to infect the permissive human A549 cells (1.5×10⁶ cells/well in a 6 well-plate) and constituted the initial virus mix. The mix used for the first passage consisted of 1.5×10⁴ PFU of each virus corresponding to a Multiplicity of Infection (MOI) of 10⁻² for each virus. One day post infection, cells and supernatant from the first initial virus mix were harvested and then used entirely to infect a confluent T-75 tissue culture flask (T75) of A549 cells (passage 1). Cells and supernatant were harvested 72 h post infection and 1 mL of this mix was used to infect a confluent T75 flask of A549 cells (passage 2). Seventy-two hours post infection, one mL of cells and supernatant from the second passage was used to infect a T75 flask of A549 cells. Supernatant from the third passage was then used in a 10-fold dilution series to infect a T75 flask of A549 cells. Amplification and selection of viral progeny was done by 6 additional passages (passage 3 to passage 9) on A549 cells by supernatant dilution. The infected T75 were observed for the first signs of cytopathic effect (CPE).

From passage 3 to passage 9, to harvest the most potent viruses, cell culture supernatant was harvested 24h post infection from the flask infected with the most concentrated inoculum in the 10-fold dilution series that did not show any sign of potent CPE. The use of the permissive A549 cell line allows a high rate of recombination between the virus resulting in the generation of more clone types, and a higher variability. From passage 9, 48 individual plaque-purified viruses were isolated, amplified, and titrated on A549 cells. Oncolytic activities of these clones were then evaluated on A549 tumor cell line.

### Generation of Variant Chimeric Poxviruses

Double (TK/RR)-deleted virus, named POXSTG20150, was generated after deletion of two short sequences within the genes encoding respectively for the RR and the TK proteins. POXSTG20150 genome nucleotide sequence was inferred by deleting in silico these two segments from POXSTG19503 genomic sequence.

### Generation of Recombinant Chimeric Poxviruses

Single (TK)-deleted virus, named POXSTG19508, was generated by insertion of the GFP::FCU1 fusion gene into the POXSTG19503 J2R locus. Briefly, CEF were infected with POXSTG19503 at a MOI of 10 2 and incubated at 37° C. for 2 h, then transfected with a shuttle plasmid containing the GFP::FCU1 fusion gene (Ricordel et al., 2017, Mol Ther Oncolytics, 7, 1-11) under the control of the synthetic p11k7.5 promoter and surrounded by the flanking sequence of the vaccinia virus J2R gene. The cells were then incubated for 48 h at 37° C. Double recombination occurred between J2R homologous regions in the shuttle plasmid and the parental virus, resulting in the insertion of the GFP::FCU1 fusion gene into the J2R locus of POXSTG19503. Recombinant virus, named POXSTG19508, was isolated and submitted to additional plaque purification cycles on CEF. Insertion of the GFP::FCU1 sequence into the J2R locus was confirmed by multiple PCRs and DNA sequencing. The same methods were used to generate the double (TK/RR)-deleted virus, named POXSTG19730, by homologous recombination between POXSTG19508 and a shuttle plasmid containing the mCherry gene under the control of the pH5R promoter and surrounded by the flanking sequence of the vaccinia virus 14L gene. Insertion of the mCherry sequence into the 14L locus was confirmed by multiple PCRs and DNA sequencing. For generation of the double (TK/RR)-deleted virus expressing murine IL-12 (mIL-12) and renamed POXSTG19847, mCherry was replaced by mIL-12 by homologous recombination between POXSTG19730 and a shuttle plasmid containing the mIL-12 gene under the control of the pH5R promoter. Insertion of the mIL-12 sequence into the 14L locus was confirmed by multiple PCRs and DNA sequencing. Single (TK)-deleted Vaccinia virus strain Copenhagen, named VVTG17111, was generated by insertion of the GFP::FCU1 fusion gene into the J2R locus. Recombinant chimeric poxviruses were amplified in CEF and purified, and virus stocks were titrated on CEFs by plaque assay.

### In Vitro Cytotoxicity Assay

The viral lytic capacity was measured using the trypan blue exclusion method. Human tumor cells were transduced in suspension by respective chimeric viruses at the indicated MOI. A total of 3×10⁵ cells/well were plated in 6-well culture dishes in 2 ml of medium supplemented with 10% FCS. Cells were then cultured at 37° C. for 4 or 5 days and the viable cells were counted by trypan blue exclusion using a Vi-Cell Cell Counter (Beckmann Coulter, CA). All samples were analysed in triplicate. Mock-infected cells served as negative control and established the 100% survival point for the given assay.

### In Vitro Virus Yield

To evaluate viral replication in human tumor cells and human primary cells, HepG2 cells and hepatocytes were infected in 6-well plates at MOI 10⁻⁵ and MOI 10⁴, respectively (in triplicate). Three days after infection, supernatants and cells were collected, freeze thawed, sonicated, and viral progeny was quantified on Vero cells by plaque assay.

To evaluate viral replication in a reconstructed human skin model, culture of Phenion full-thickness skin model was infected with the indicated virus at 10⁵ PFU (in triplicate). Cultures were incubated for 7 days at 37° C. Viral progeny in reconstructed skin were quantified on CEFs by plaque assay after 2 cycles of sonication in PBS.

To quantify the infectious EEV and IMV at early times after infection, confluent A549 cells (1×10⁶ cells) were infected in 6-well plates at MOI 10⁻¹ for 1 h (in triplicate). Cells were then washed with PBS and replaced with fresh medium and incubated for 16 or 24 hours. Supernatants and cell fractions were collected separately. The supernatants were used to quantify EEV. The cell fractions were harvested in 1 mL PBS and the cell-associated virions, namely IMV, were extracted from cell lysates by freeze-thawing and sonication. Both virions (EEV and IMV) were titrated on CEFs by plaque assay.

### Cytosine Deaminase Enzymatic Assay

Cytosine deaminase (CDase) activity was quantified by measuring the amount of 5-FU released in the culture media. A549 cells were infected with the different vectors at a MOI of 10⁻⁴ (in triplicate) and plated in 6-well culture dish (3×10⁵ cells/well). After 6 hours, 1 mM 5-FC was added to the culture medium. From day 1 to day 3 post infection, 5-FC and 5-FU concentrations in the media were measured by HPLC. Fifty μl of media were quenched with 50 μL of acetonitrile. The samples were vortexed and centrifuged. The organic supernatant was evaporated to dryness and reconstituted in 50 μL of water and analysed by HPLC using a mobile phase of 50 mM phosphoric acid adjusted to pH 2.1. Results are expressed as the percentage of 5-FU relative to the total amount of 5FC+5FU after various incubation times with 5-FC.

### Comet Assays

A549 cells were seeded in 60 mm tissue culture dishes and grown to confluence overnight. The next day, cell monolayers were rinsed with PBS, inoculated with approximately 40 plaque-forming units (PFU) of viruses in 500 μL of PBS supplemented with 2% FCS, 1% cations, and incubated for one hour at 37° C. After one hour, monolayers were rinsed and overlaid with 5 ml of growth media. Two days after infection, plaques were imaged using a Nikon SMZ18 fluorescence microscope (GFP) and monolayers were stained with crystal violet (stock solution diluted 1:40 in ethanol) for visualisation.

### Neutralization Assay

Viruses' oncolytic activity was performed in the presence of serum collected from patient before and after IV injection of TG6002. HCT116 tumor cells seeded in 96-well plate (1×10⁴ cells/well) were infected with the indicated viruses at various MOIs in triplicate. Virus inoculums were incubated with serum (diluted 100-folds in PBS) for 1 h at 37° C. before being added to the cells. At 96 h post-infection, the viability of tumor cells was determined using the CellTiter-Blue Cell Viability Assay (Promega) following the manufacturer's instruction. Optical density was read at a microplate reader (Tecan Infinite M1000 Pro) with 560 nm excitation and 590 nm emission. The viability of the infected tumor cells was calculated as a percentage relative to the mock-infected cells. The EC50 was determined by fitting a sigmoidal dose-response curve using the GraphPad Prism software.

### Animal Studies

For human xenograft tumor models, 5×10⁶ human cancer cells (HCT116 and HepG2) were injected subcutaneously into the flank of Swiss nude mice. When the tumors reached a diameter of 100-200 mm³, the mice were randomized in a blinded manner and treated once intravenously with the indicated vectors.

For bilateral tumor model, 5×10⁶ human cancer HCT116 cells were injected on both sides of the flank of Swiss nude. When the tumors reached a diameter of 100-200 mm³, the mice were randomized in a blinded manner and the virus was injected once intratumorally in the right-side tumor.

For syngeneic murine tumor models, 2×10⁵ CT26 cells or 3×10⁵ B16F10 cells were injected subcutaneously into the flank of BALB/c mice (CT26) or C57BL/6 mice (B16F10). When tumors became palpable, the mice were randomized in a blinded manner and treated intratumorally with the indicated vectors.

Tumor size was measured twice weekly using calipers. Tumor volumes were calculated in cubic millimeters using the formula π/6×length×width². The animals were euthanized when their tumor volumes reached 2000 mm³. For bilateral tumor model, mice were euthanized when total volume of both tumors exceeded 2000 mm³.

To assess the amount of virus in tumors, tumors were collected and weighed, homogenized in PBS, sonicated, and titers were determined on Vero cells by plaque assay.

### Immunohistochemical Analysis

Tumors were collected, formalin fixed, paraffin-embedded and sectioned. Sections (5 μM) were mounted on adhesive glass slides and used for histological analysis. Virus-infected cells were detected upon incubation of the slide with rabbit IgG anti-vaccinia virus (B65101R, Meridian) at a dilution of 1:1400 and the antibody-binding signal was detected by using the NovoLink Polymer Detection System (Leica Microsystems). Signals were visualized with the TSA-fluorescein pack (SAT701001EA, Akoya) and counterstained with DAPI (B-2883, Sigma). Stained slides were scanned by a high-resolution fluorescent slide scanner (Nanozoomer, Hamamatsu) and quantification of the signal was done using the Calopix software (Tribvn). Virus-infected cells were expressed as a proportion of total tumor area.

### Syncytia Formation Assay

Vaccinia virus strain Copenhagen and chimeric poxvirus were tested in the HCT116 human CRC cancer cell line. HCT116 cells were infected with the VVTG17111 and POXSTG19508 at a MOI of 10⁻² or 10⁻³ and plated in 6-well culture dish (3×10⁵ cells/well). Cells were observed by optical and fluorescence microscopy at 40 and 60h post infection.

### Neutralization Assay by Complement-Mediated Virus

Vaccinia virus strain Copenhagen and chimeric poxvirus were incubated for 1 hour at 37° C. at a dose of 2×10⁷ PFU in 200 μL of commercially available normal human serum (Sigma-Aldrich, Darmstadt, Germany). The infectious virus remaining was quantified by plaque assay on Vero cells. As a control for complement activation, viruses were incubated under the same conditions with heat-treated human serum at 56° C. for 1 hour and quantified as described above.

### Specific T-Cell Response Measured by IFNγ ELISpot

Interferon-γ (IFN-γ) secretion by splenocytes T cell from mice treated with viruses was assessed by ELISpot assays to evaluate the activation of the cytotoxic T lymphocytes CTLs against both tumoral and viral validated epitopes. Murine colon carcinoma CT26 cells (2×10⁵ cells) were injected subcutaneously into the flank of BALB/c mice. When tumors became palpable, the mice were randomized in a blinder manner and treated intratumorally with a single injection of VVTG17111 or POXSTG19508 at 1×10⁷ PFU. Six days post-injection, mice were euthanized, and their spleens were collected. Mononuclear cells were obtained from splenocytes by density gradient centrifugation on Lymphocyte-M (Cedarlane) and after red blood cell lysis (BD Pharm Lyse Lysing Buffer 1×). 350,000 cells/well were plated in 96-well MSIP plates (Millipore) coated with anti-IFNγ capture mAB (Mabtech) and stimulated with 1 ug/mL of an irrelevant peptide (TPHPARIGL) or with 1 μg/ml of the vaccinia-specific S9L8 peptide (SPGAAGYDL) or with 1 μg/mL of the tumor-associated antigen AH1 peptide (SPSYVYHQF). Plates were incubated for 18 hours, washed and immunospots were revealed using biotinylated anti-IFNγ detection mAB (Mabtech AB, Extravidin-Phosphatase alkaline (Sigma) and BCIP/NBT solution (Sigma). Spots were counted using an ELISpot reader (CTL Immunospot reader, S5UV).

### DNA Sequencing

Samples from viral DNA were purified with AMPure XP (Beckman Coulter, Inc.) beads kit to remove residual cellular DNA and sent for sequencing to the GenomEast platform (IGBMC Microarray and Sequencing platform, Illkirch-Graffenstaden, France). Data from Illumina HiSeq4000 in 2×100 bp paired-end runs were quality trimmed using custom scripts written in Perl and R. Pairs with at least one read having more than 5 bases with a Phred quality score <13 were discarded at this step. The filtered reads were then mapped (using bwa, doi: 10.1093/bioinformatics/btp324) against the genome of the host cells used for virus production, and the pairs properly mapped with a high mapping score were considered as contaminant and discarded. This step was particularly important for samples with high proportion of contaminating DNA from host cell genomes. Contigs were de novo assembled with SPAdes v3.11.1 (Nurk et al., 2013, J Comput Biol., 20, 714-737. doi: 10.1089/cmb.2013.0084) following authors instructions and scaffolding was performed with custom script to generate the longest consensus sequence for each viral genome. Open reading frames (ORFs) longer than 150 nt were automatically annotated, and a custom script written in python3 and based on the Dijkstra algorithm was used to identify homologous regions between POXSTG19503 and each of the parental strains. Global pairwise alignments were performed using MAFFT v7.017 to measure the genomic homology between POXSTG19503 and each parental genome, and to highlight the longest regions with strict identity between POXSTG19503 and the corresponding parental genome (Katoh et al., 2002, Nucleic Acids Res. 30, 3059-3066). In addition, multiple alignments of the whole core-region of POXSTG19503 and the parental genomes was performed using Mauve (doi: 10.1101/gr.2289704) to identify potential structural variants.

### Results

POXSTG 19503 is a Chimeric Poxvirus with Enhanced Oncolytic Potency In Vitro

A direct evolution strategy was employed to generate chimeric poxvirus with increased oncolytic potency and tumor selectivity. A library of viruses was first generated by co-infecting A549 cells with 16 poxviral strains including VVs, CPXV, RCNV, RPXV, BPSV, ORFV, PCPV, MYXV, YLDV, SWPV, CTV, SQFV and FPV. Then, amplification of viral progeny under stringent conditions was performed by nine consecutive passages on A549 cells. The use of the permissive A549 cell line allows a high rate of recombination between the virus resulting in the generation of more clone types, and a higher variability. Moreover, in order to select chimeric viruses with high oncolytic potential, several passages were carried out using high dilutions (1/10 000) as well as short post-infection collection times (24h). Forty-eight individual plaque-purified viruses were isolated from passage 9 and screened for their oncolytic potential on a panel of tumor cells. With one of the clones, designated POXSTG19503, we have obtained a superior oncolytic activity on the 6 tested tumor cell lines (A549, MIA PaCa-2, U-87 MG, B16F10 and HepG2) compared to COP(FIG. 1) which had shown the highest oncolytic activity of the parental strains (Ricordel et al., 2018, Cancers (Basel), 10, doi: 10.3390/cancers10070231; Ricordel et al., 2018, Oncotarget 9, 35891-35906). The clone purity was assessed by evaluation of 10 plaque-purified clones originated from POXSTG19503, all of which displayed the same phenotype and oncolytic activity in different tumor cells (data not shown).

### Genome Analysis

DNA from POXSTG19503 and from the 16 parental virus species or strains were purified and sequenced by Next Generation Sequencing. The use of paired-end short reads and whole genome de novo assembly led to the generation of viral genomes with the core region and one copy of the Inverted Terminal Repeats (ITR) domain. These single-ITR versions of the genomes are due to the use of unique k-mers in the contigs assembly process, but the presence of both ITR regions in every viral genome was confirmed by the mean depth of coverage which was higher than the one from the core region, with a 2-fold factor (data not shown). Single-ITR versions of the genomes were retained for the sake of clarity in the subsequent paragraph. Length of resulting genomes sequences for the parental viruses and POXSTG19503 (185′577 nt) are reported in Table 2.

TABLE 2
Size of the de novo assembled genomes and ITR. Virus short name
and full names are indicated in the two first columns. Size of the
mono-ITR genome and the assembled ITR only are reported in the
3^{rd} and 4^{th} column, respectively.

| | | Size of final | Size of |
|---|---|---|---|
| | | assembled | assembled |
| Virus ID | Virus name | genome | ITR |
| COP | Vaccinia virus | 175′860 | 8′158 |
| WR | Vaccinia virus | 181′419 | 6′938 |
| Wyeth (WY) | Vaccinia virus | 182′664 | 16′306 |
| MVATG15938 | Vaccinia virus | 163′444 | 3′988 |
| CPX | Cowpox virus | 212′521 | 6′507 |
| RCN | Raccoonpox virus | 201′260 | 1′737 |
| RPX | Rabbitpox virus | 186′491 | 3′462 |
| ORF | Orf virus | 133′176 | 2′032 |
| PCP | Pseudocowpox | 128′008 | 12′426 |
| BPS | Bovine Papular | 133′871 | 863 |
| | stomatitis virus | | |
| MYX | Myxoma virus | 150′327 | 11′604 |
| SqPV (SF) | Squirrelpox virus | 147′605 | No ITR |
| FPV (FP) | Fowlpox virus | 248′557 | 6′500 |
| SPV (SWP) | Swinepox virus | 122′765 | 3′597 |
| YLD | Yaba-like | 142′724 | 1′915 |
| | disease virus | | |
| CTV (COT) | Cotia virus | 158′746 | 939 |
| POXSTG19503 | Chimeric poxvirus | 185′577 | 9′667 |
| POXSTG19508 | Chimeric poxvirus | 187′589 | 9′667 |
| | TK- encoding | | |
| | GFP::FCU1 | | |
| POXSTG19730 | Chimeric poxvirus | 188′046 | 9′667 |
| | TK- RR- encoding | | |
| | GFP::FCU1 | | |
| | and mCherry | | |
| POXSTG20150 | Chimeric poxvirus | 184′661 | 9′667 |
| | TK- RR- | | |

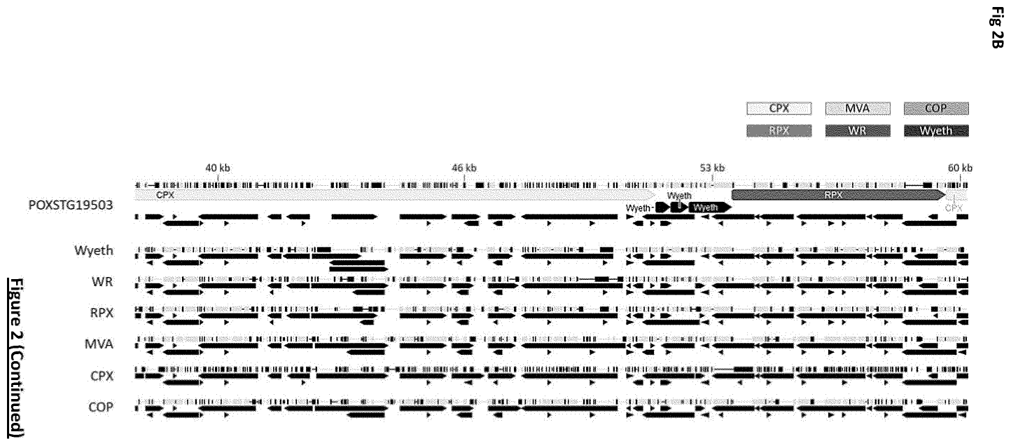

Sequence comparison between de novo genome assemblies showed that almost each segment of POXSTG19503 genome was 100% identical to at least one segment from one parental virus. However, sequences from only 6 parental viruses, namely Rabbitpox virus, Cowpox virus, and the Vaccinia virus strains Wyeth, Western-Reserve, Copenhagen and the Modified Vaccinia Virus Ankara, were found 100% identical in POXSTG19503. These 6 viruses belonged all to the Poxvirus genus, indicating that homologous recombination occurred preferentially between the closely related members of this genus. Segments of POXSTG19503 genome longer than 500 nucleotides and 100% identical of a parental genome are indicated on FIG. 2A and reported in Table 3. Of note, the longest segment identified was 100% identical to the cowpox virus genome on 37′000 nucleotides (position 14,242 to 51,241).

TABLE 3
Genomic coordinates and length of POXSTG19503 segments that
are 100% identical to a parental genome. Name of the parental
genome is indicated in the first column, coordinates in the 2^{nd} and
3^{rd} columns and length of the segments are reported in the
last column. The ″start position″ and ″stop position″
correspond to nucleotide positions. The nucleotide in position
1 is defined as the first nucleotide of the core genome.

| Parental virus | Start pos | Stop pos | Length (nt) |
|---|---|---|---|
| CPX | 14,242 | 51,241 | 37,000 |
| MVA | 95,530 | 116,249 | 20,720 |
| RPX | 137,520 | 154,979 | 17,460 |
| CPX | 59,852 | 72,141 | 12,290 |
| MVA | 118,460 | 127,289 | 8,830 |
| RPX | 54,042 | 59,851 | 5,810 |
| RPX | 83,610 | 88,879 | 5,270 |
| RPX | 154,990 | 160,079 | 5,090 |
| RPX | 562 | 4,701 | 4,140 |
| Wyeth | 176,100 | 179,909 | 3,810 |
| RPX | 127,290 | 130,589 | 3,300 |
| MVA | 134,800 | 137,519 | 2,720 |
| Wyeth | 81,060 | 83,529 | 2,470 |
| WR | 169,190 | 171,579 | 2,390 |
| MVA | 91,460 | 93,839 | 2,380 |
| WR | 173,730 | 176,099 | 2,370 |
| Wyeth | 162,290 | 164,599 | 2,310 |
| Wyeth | 116,250 | 118,459 | 2,210 |
| Wyeth | 181,920 | 184,099 | 2,180 |
| MVA | 88,880 | 90,899 | 2,020 |
| Wyeth | 76,630 | 78,639 | 2,010 |
| RPX | 131,230 | 133,199 | 1,970 |
| Wyeth | <74103 | 75,719 | >1617 |
| RPX | 133,200 | 134,799 | 1,600 |
| Wyeth | 165,670 | 167,259 | 1,590 |
| Wyeth | 52,472 | 54,041 | 1,570 |
| Wyeth | 78,640 | 80,079 | 1,440 |
| Wyeth | 180,020 | 181,289 | 1,270 |
| Wyeth | 72,772 | 74,021 | 1,250 |
| Wyeth | 160,080 | 161,239 | 1,160 |
| Wyeth | 10,112 | 11,261 | 1,150 |
| WR | 172,550 | 173,659 | 1,110 |
| Wyeth | 164,600 | 165,669 | 1,070 |
| Wyeth | 6,522 | 7,581 | 1,060 |
| WR | 80,080 | 81,059 | 980 |
| Wyeth | 168,210 | 169,189 | 980 |
| WR | 167,260 | 168,209 | 950 |
| WR | 171,590 | 172,539 | 950 |
| COP | 7,612 | 8,521 | 910 |
| CPX | 75,720 | 76,629 | 910 |
| MVA | 184,160 | 185,029 | 870 |
| MVA | 94,670 | 95,519 | 850 |
| Wyeth | 93,840 | 94,669 | 830 |
| Wyeth | 5,702 | 6,511 | 810 |
| Wyeth | 8,652 | 9,451 | 800 |
| Wyeth | 51,742 | 52,471 | 730 |
| WR | 11,812 | 12,451 | 640 |
| Wyeth | 5,112 | 5,691 | 580 |
| Wyeth | 161,340 | 161,919 | 580 |
| RPX | 181,290 | 181,859 | 570 |
| Wyeth | 90,910 | 91,459 | 550 |
| Wyeth | 62 | 561 | 500 |
| Wyeth | 9,612 | 10,111 | 500 |
| Wyeth | 51,242 | 51,741 | 500 |
| Wyeth | 130,730 | 131,229 | 500 |

To further investigate the composition of POXSTG19503 genome, the percentage of nucleic acid identity was explored by global pairwise alignments with the genome of parental viruses, using Mafft (Table 3). Nucleic acid sequence of POXSTG19503 core region was 91.8% identical to COP, 84.6% identical to CPX, 86.5% identical to MVA, 94.8% identical to RPX, 96.5% identical to WR and 94.9% identical to Wyeth.

TABLE 4
Percentage of nucleic acid identity between
POXSTG19503 and the 6 parental genomes

| | COP | CPX | MVA | RPX | WR | Wyeth |
|---|---|---|---|---|---|---|
| POXSTG19503 | 91.8 | 84.6 | 86.5 | 94.8 | 96.5 | 94.9 |

Multiple alignment of the core-genome region of POXSTG19503, RPX, CPX, WR, Wyeth, COP and MVA (with Mauve) showed the almost perfect conservation of the genome organization, with no structural variant such as large insertion, deletion or inversion detected when comparing POXSTG19503 to the parental genomes (FIG. 2B). Consequently, almost all the Open Reading Frames and their relative positions in the viral genomes were also conserved in POXSTG19503.

### Deletion of J2R Gene Improves Efficacy and Safety of the Chimeric Poxvirus

The coding sequence of GFP-FCU1 (GFP::FCU1) fusion gene was introduced into the J2R (TK) locus of POXSTG19503 to generate the thymidine-deleted chimeric poxvirus POXSTG19508. The GFP::FCU1 fusion protein exhibits cytidine deaminase (CDase) and uracil-phosphoribosyltransferase (UPRTase) activities similar to those of the FCU1 protein and displays a fluorescent signal intensity equivalent to the one of native eGFP protein (Ricordel et al., 2017, Molecular Therapy Oncolytics, 7:1-11).

Oncolytic activities of the thymidine-deleted chimeric poxvirus POXSTG19508 and the thymidine-deleted parental strains (VACV strains Copenhagen, Western Reserve, Wyeth, MVA, Cowpox virus strain Brighton and Rabbitpox virus strain Utrecht) were compared in A549 (FIG. 3A), HCT116 (FIG. 3B) and HepG2 (FIG. 3C) tumor cells. The viral efficacy of the chimeric poxvirus was not affected by TK knockout. Moreover, POXSTG19508 showed better oncolytic efficacy than all 6 parental poxvirus strains indicating that virus chimerization can generate a backbone virus that is more efficient than its parental viruses.

Most of the following comparisons were made against the Vaccinia virus strain Copenhagen. In fact, Rabbitpox viruses are too virulent to be used as oncolytic viruses for treating human cancers. They were, however, used as comparators for EEV production, as they are known to be good EEV producers.

Cowpox viruses showed a too weak oncolytic power in vivo to be an interesting comparison element. Moreover, in vitro and in vivo preclinical studies have recently shown that the Copenhagen strain has a more potent oncolytic viral activity against human tumor cells than the Wyeth and the WR strains (Foloppe et al., 2019, Mol. Ther. Oncolytics Vol.14, 1-14), and MVA is known to be not oncolytic.

Compared to COP wild-type, the chimeric wild-type POXSTG19503 produced more viral particles in HepG2 tumor cells (FIG. 4A). The chimeric wild-type POXSTG19503 also demonstrated a reduced replication on primary cells (FIG. 4A): as compared to COP wild-type, 2-fold and 4-fold reduction of replication were observed respectively in human reconstituted skin model and hepatocytes. Consequently, the specificity index calculated from the ratio between viral fold amplification obtained in HepG2 hepatocarcinoma cells and hepatocytes was largely improved for the chimeric poxvirus POXSTG19503 (FIG. 4B).

Moreover, J2R deletion had a clear benefit on POXSTG19508 allowing a supplementary 8-fold reduction of replication in human skin and hepatocytes (FIG. 4A). These results are consistent with previously reports demonstrating the benefit of deleting J2R gene for reducing virus replication of poxviruses in non-cancerous cells (Foloppe et al., 2019, Mol Ther Oncolytics, 14, 1-14; Ricordel et al., 2017, Molecular Therapy Oncolytics, 7:1-11; Ricordel et al., 2018, Cancers (Basel), 10, doi: 10.3390/cancers10070231; Ricordel et al., 2018, Oncotarget 9, 35891-35906).

Therefore, the ratio of viral progeny produced on HepG2 tumor cells to that in human primary hepatocytes, was largely improved for POXSTG19508 where the transgene was inserted into the J2R locus (FIG. 4B).

### POXSTG19508 Shows Superior CDase Activity Relative to Parental Virus

POXSTG19508 expressed the therapeutic gene FCU1, fused with eGFP, that catalyzes the conversion of 5-FC into 5-FU and its derivates metabolites (Erbs et al, 2000, Cancer research, 60, 3813-3822). Expression of functional FCU1 protein by POXSTG19508 was confirmed by quantification of 5-FU released into the supernatant of infected cells and was compared to the expression of FCU1 encoded by the thymidine-deleted vaccinia virus strain Copenhagen (VVTG17111) (FIG. 5). The analysis of A549 cells supernatant by HPLC showed a progressive release of 5-FU into the extracellular medium of cells infected with the indicated viruses at a MOI of 10⁻⁴ and incubated with 1 mM 5-FC. Two and 3 days after infection with the virus VVTG17111, respectively 15% and 60% of 5-FC was converted to 5-FU in the supernatant. At these same collection times, more than 40% and 90% of 5-FC was deaminated to 5-FU in the supernatant of POXSTG19508 infected cells, indicating higher expression of FCU1 following increased replication of the chimeric poxvirus in tumor cells.

### Chimeric Poxvirus Produces More EEV and Forms Comets

We analysed the production of EEV and IMV in A549 cancer cells (FIGS. 6, 7 and 8). A549 cells were seeded in six-well plates and the next day infected with the thymidine-deleted vaccinia virus strain Copenhagen (VVTG17111) and the thymidine-deleted chimeric poxvirus POXSTG19508 at MOI of 0.1. At 16 and 24 hours after infection, supernatant (for EEV) or cell fraction (for IMV) were harvested, and infectious viruses were quantified by plaque assays on CEFs. At different time post-infection (16 and 24 hours), the yields of total virus (both IMV and EEV) from POXSTG19508 were two-fold higher than VVTG17111, whereas the yields of EEV form produced from POXSTG19508 were 20- to 40-fold more than that from VVTG17111 (FIG. 6A, FIG. 6B). These results showed that the ratio of EEV to IMV was around 0.5% for the vaccinia virus strain Copenhagen (FIG. 6C), consistent with published results (Spehner et al., 2000, Virology, 273, 9-15, doi: 10.1006/viro.2000.0411). For the chimeric poxvirus, this ratio was markedly increased to over 5% and 10% at 16 and 24 hours, respectively (FIG. 6C).

The amount of EEV released by infected cells into the culture medium can be qualitatively monitored by a standard plaque assay performed under liquid overlay. In this assay, viruses that release large amounts of EEV give rise to plaques with a characteristic comet shape (Smith et al., 1998, Adv Exp Med Biol, 440, 395-414). The comet tail is believed to form by small secondary plaques derived from EEV released by the primary infected cell. As expected, the chimeric poxvirus POXSTG19508, which produced large amounts of EEV, forms comet-shaped virus plaques under liquid overlay while the parental vaccinia virus strain Copenhagen does not (FIG. 7).

A549 cells were seeded in six-well plates and the next day infected with the Vaccinia Virus strain IHD-J (a strain known to form high level of EEV in supernatant), the thymidine-deleted rabbitpox virus encoding GFP (RPXTG19095) or the thymidine-deleted chimeric poxvirus POXSTG19508 at MOI of 0.1. At 16 and 24 hours after infection, supernatant (for EEV) or cell fraction (for IMV) were harvested, and infectious viruses were quantified by plaque assays on CEFs. At different time post-infection (16 and 24 hours), the yields of total virus (both IMV and EEV) from POXSTG19508 were higher than RPXTG19095, whereas the yields of EEV form produced from POXSTG19508 were higher than that from RPXTG19095 (FIG. 8A, 8B). These results showed that the ratio of EEV to IMV was around 3% and 4% for the Vaccinia Virus strain IHD-J at 16 and 24 hours, respectively, around 5% and 4% for the rabbitpox virus at 16 and 24 hours, respectively. For the chimeric poxvirus, this ratio was markedly increased to over 6% and 12% at 16 and 24 hours, respectively (FIG. 8C).

### Chimeric Poxvirus Escapes Neutralization by Immunized Serum.

To compare neutralization resistance, immunized serum from patient treated by TG6002 was mixed with respectively TG6002 or POXSTG19508, and cytotoxicity of the escaped viruses were evaluated. As expected, TG6002 was strongly neutralized by immunized serum (FIG. 9A), resulting in an increase in EC50 (FIG. 9C). In contrast, the chimeric poxvirus POXTG19508 escaped neutralization at the same concentration of immunized serum (FIG. 9B) without significant modification in the EC50 value (FIG. 9C).

### Chimeric Poxvirus Shows Enhanced Capacity for Tumoral Spread.

We evaluated the ability of the viruses to propagate in the tumor after either a systemic injection in a xenograft model or after an IT injection in a syngeneic model. In the xenograft HCT116 model, virus staining was similar 2 days after IV injection of VVTG17111 and POXSTG19508 with about minus 2% positive tumor area. Following replication of VVTG17111, virus staining increases to reach approximately 10% to 20% of tumor area one to two weeks post-injection. Patches of virus staining increase more strongly for POXSTG19508 to reach more than 50% of tumor area indicating more pronounced tumor spreading of the chimeric poxvirus in human tumors (FIG. 10A, FIG. 10B). In the syngeneic B16F10 model, vaccinia was weak (D3 post-injection) or absent (D8 post-injection) after IT injection of VVTG17111 and staining for vaccinia antigen was strong in tumors injected with POXST19508 indicating the increased capacity for intratumoral spreading of the chimeric poxvirus in murine tumors (FIG. 11A, FIG. 11B).

### Chimeric Poxvirus Shows Enhanced Oncolytic Potency in Various Tumor Mouse Models.

We compared the oncolytic activity of the chimeric poxvirus POXSTG19508 and TG6002 in xenograft and syngeneic tumor models.

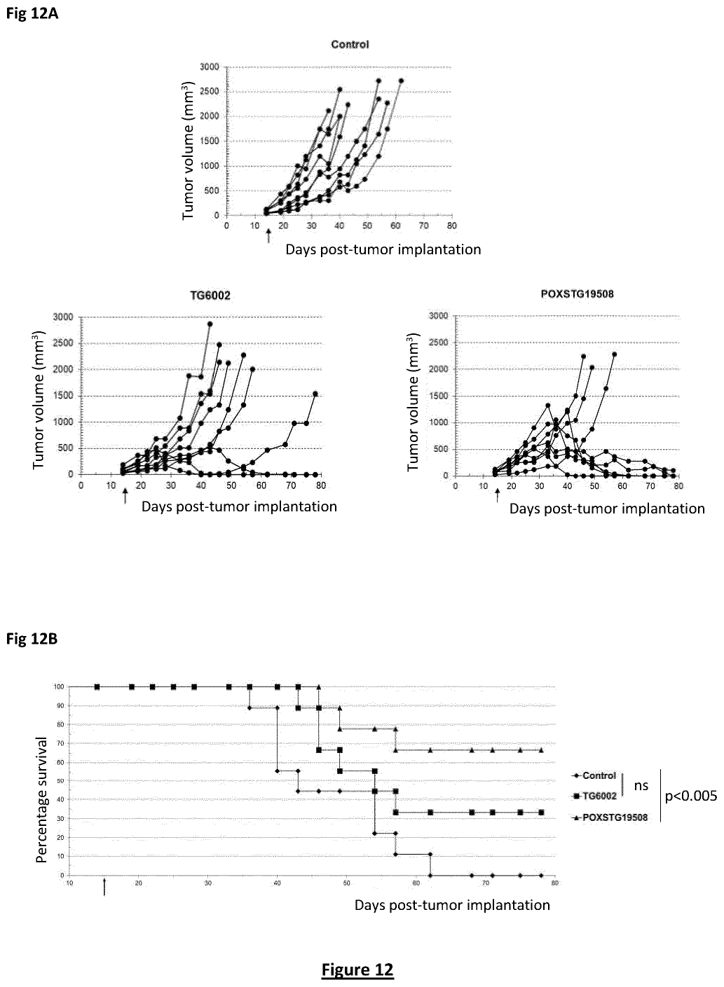

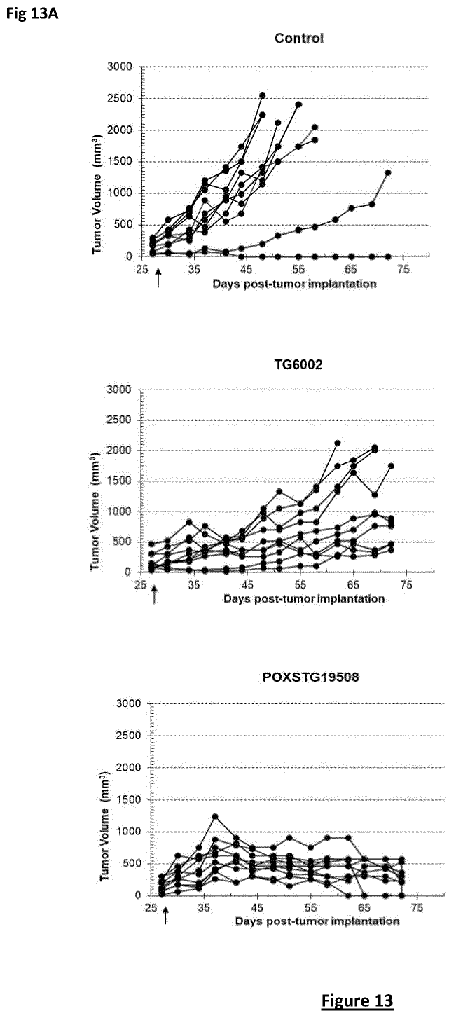

Nude mice bearing HCT116 tumors were injected intravenously with virus TG6002 and POXSTG19508, both at 3×10⁴ PFU (suboptimal dose of TG6002 for HCT116 model). As shown in FIG. 12A, a single intravenous injection of POXSTG19508 resulted in a superior inhibition of tumor growth (reduction in tumor volume for 6 out of 9 tumors 78 days post-tumor implantation) compared to TG6002 (reduction in tumor volume for 3 out of 9 tumors 78 days post-tumor implantation). As shown in FIG. 12B, comparison of survival curves showed that POXSTG19508 increased mouse survival compared to TG6002.

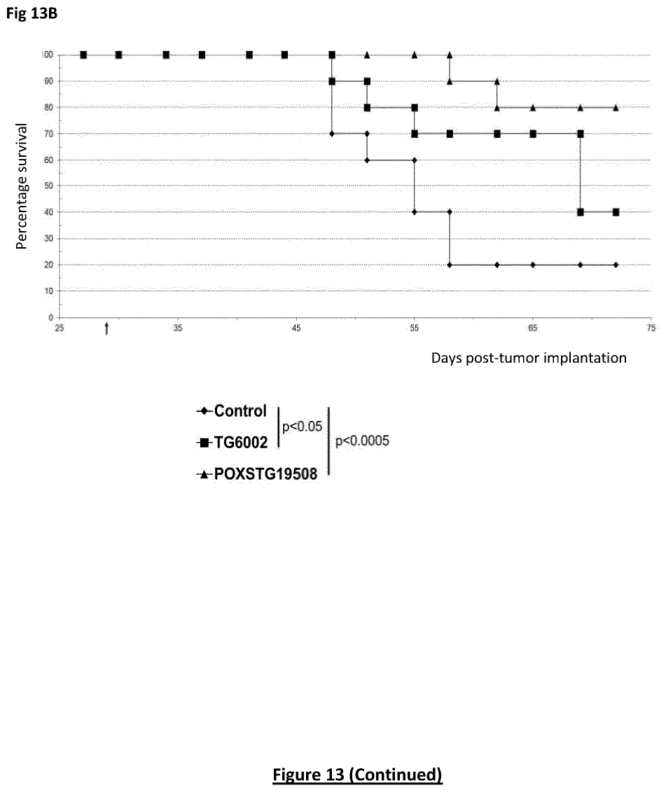

Nude mice bearing HepG2 tumors were injected intravenously with virus TG6002 and POXSTG19508, both at 3×10⁵ PFU (suboptimal dose of TG6002 for HepG2 model). As shown in FIG. 13A, a single intravenous injection of POXSTG19508 resulted in a superior inhibition of tumor growth compared to TG6002. As shown in FIG. 13B, comparison of survival curves showed that POXSTG19508 increased mouse survival compared to TG6002.

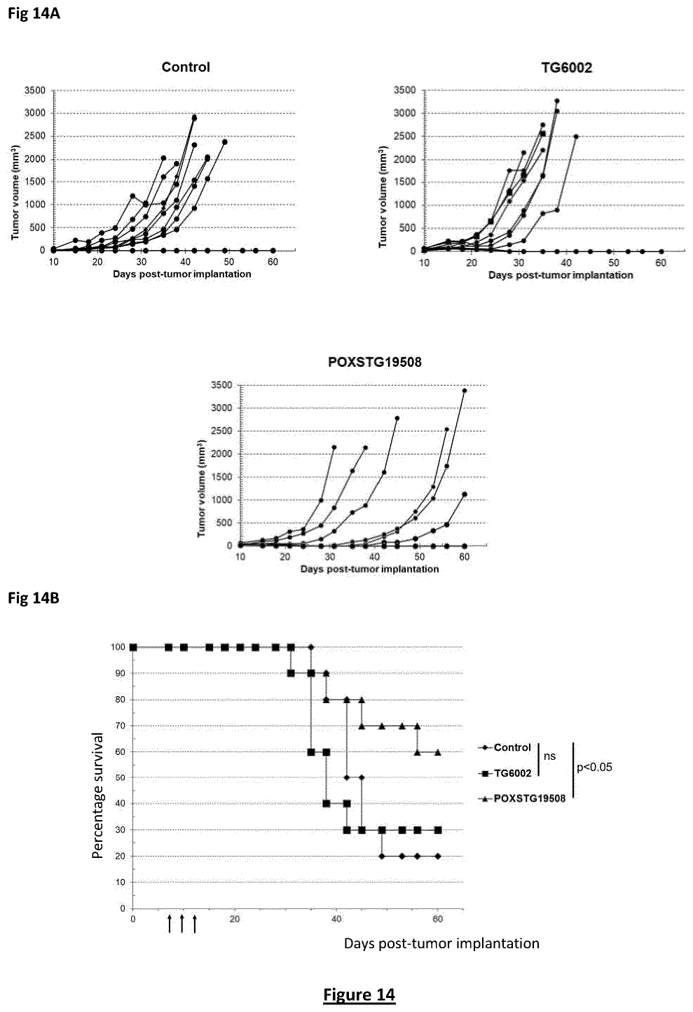

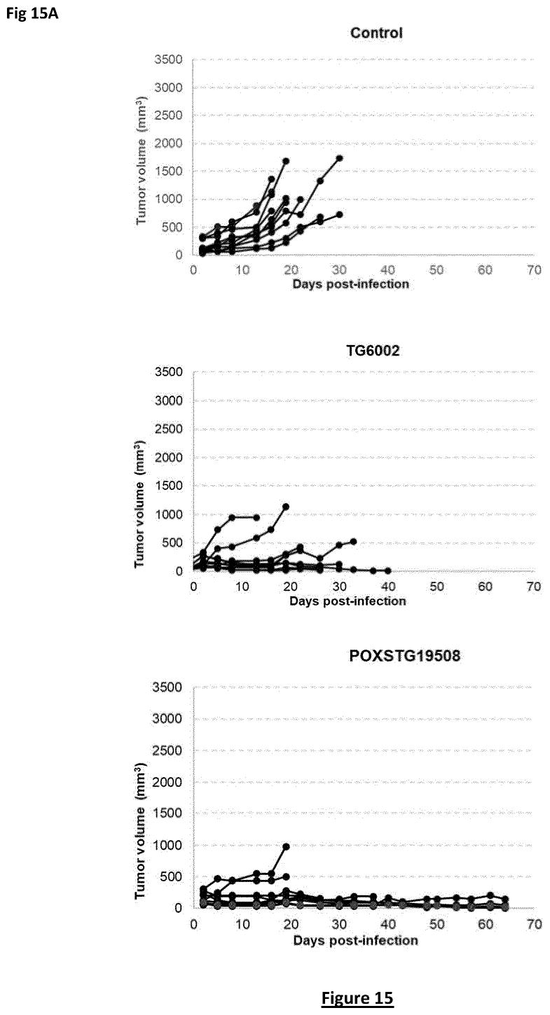

Because murine tumors are less susceptible to oncolytic vaccinia virus, the two viruses were injected three times intratumorally and at high dose (1×10⁷ PFU) in mice bearing murine CT26 tumors, known for being resistant to Vaccinia viruses. As expected, no tumor growth control was obtained using TG6002 in this model, whereas treatment with POXSTG19508 slowed tumor progression (FIG. 14A), resulting in an increase in mice survival (FIG. 14B).

### Chimeric Poxvirus Efficiently Spreads to Distant Tumors in a Human Tumor Xenograft

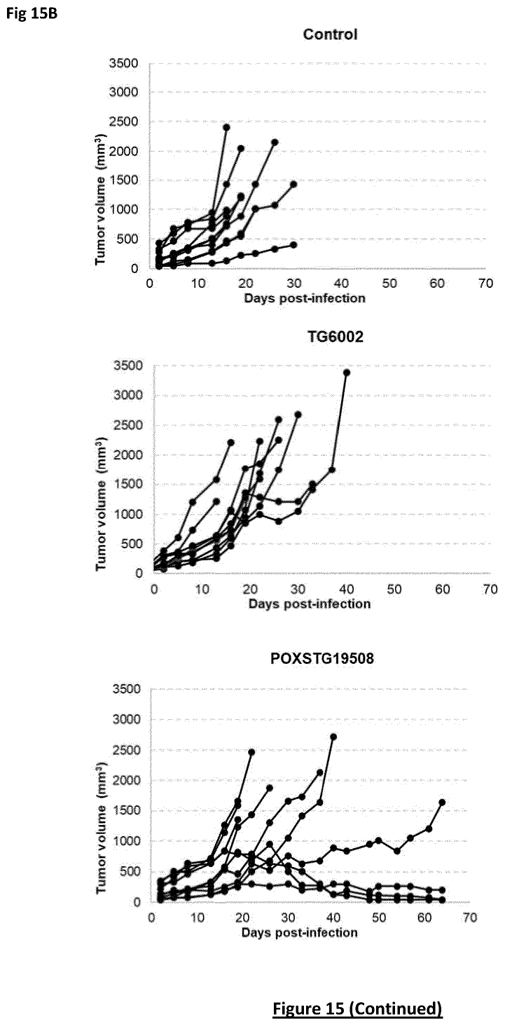

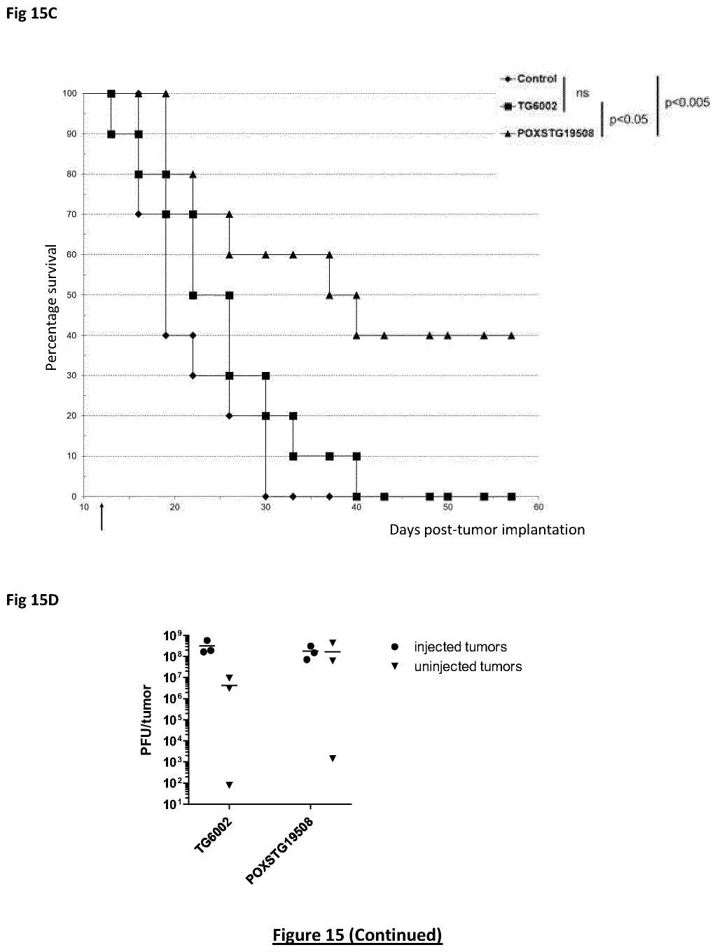

Nude mice bearing bilateral HCT116 xenografts in their flanks were injected intratumorally with PBS or 10⁶ PFU of TG6002 or POXST19508 in only one of the two tumors. A single intratumoral injection of TG6002 and POXST19508 induced strong antitumor effect in injected tumors (FIG. 15A).

Moreover, unlike TG6002, POXST19508 was able to disseminate to distant uninjected tumors and showed antitumor effect in these tumors (FIG. 15B). More importantly, POXST19508 treatment significantly increased the survival of mice compared to PBS or TG6002 treated group (FIG. 15C). In a parallel experiment, three mice per group were euthanized on day 13 after virus injection, and virus titers in tumors were determined. Equivalent amounts of the two viruses were detected in injected tumors indicating the replication of the viruses in the tumors (FIG. 15D). However, compared to TG6002, higher titers of POXST19508 were detected in uninjected tumors, confirming superior spread of the chimeric poxvirus (FIG. 15D).

### Similar Properties of a Double Deleted TK-RR-Variant

The oncolytic power, viral replication on primary cells, therapeutic index, EEV-secretion capacity, spreading capacity and neutralization rate in the presence of anti-poxvirus antibodies of a variant chimeric poxvirus defective in J2R locus and 14L locus (POXSTG19730) were evaluated and compared to the corresponding functions of the variant parental COP defective in J2R locus and 14L locus. The obtained results showed that the further deletion in 14L locus had no deleterious impact on the above-mentioned functionalities. Furthermore, compared to what was observed for the variant viruses defective in the J2R locus only, the hierarchy of results was respected: the variant chimeric poxvirus defective in J2R locus and 14L locus had a higher oncolytic power, a lower viral replication on primary cells, a higher therapeutic index, a higher EEV-secretion capacity, a higher spreading capacity, and a lower neutralization rate in the presence of anti-poxvirus antibodies than the variant parental COP defective in the J2R locus and 14L locus (data not shown).

Systemic Administration of the Chimeric Poxvirus Expressing mIL-12 Shows Significant Antitumor Activity in the Syngeneic B16F10 Mouse Model.

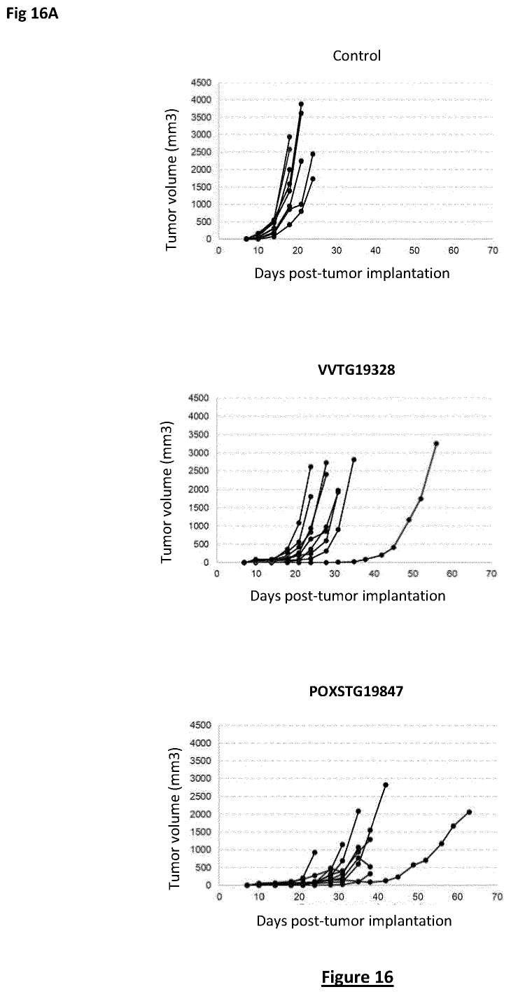

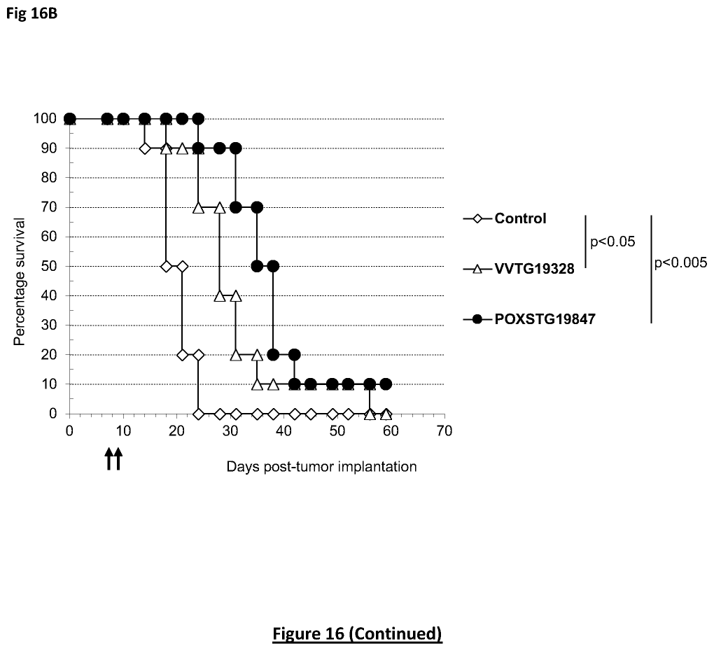

To improve the in vivo efficacy associated with the VACV and the chimeric poxvirus, the immunomodulatory cytokine murine IL-12 (mIL-12) was incorporated into the 14L region of the viruses under the control of the pH5R promoter. The antitumor effect of the double (TK/RR)-deleted viruses, expressing or not murine IL-12, was evaluated in syngeneic mouse model. Immunocompetent mice with subcutaneous murine B16F10 tumors were treated with either PBS, double deleted VACV expressing mIL-12 (VVTG19328) and double deleted chimeric poxvirus expressing mIL-12 (POXSTG19847) via IV delivery on day 7 and 9 after tumor implantation. This is an aggressive tumor model, however, IV administration of the chimeric poxvirus expressing mIL-12 (POXSTG19847) was able to slow tumor growth (FIG. 16A) and also enhanced survival compared with VACV expressing mIL12 (FIG. 16B).

### Chimeric Virus Induced Syncytia Formation

Vaccinia virus strain Copenhagen (COP) or chimeric poxvirus were used to infect HCT 116 human cancer cells, and the cell-to-cell fusion was observed by optical and fluorescence microscopy. Infection with the thymidine-deleted Vaccinia virus strain Copenhagen (VVTG17111) resulted in an altered cell morphology, with individual and compartmentalized round cells with no fused cells (FIG. 17). In contrast to the thymidine-deleted Vaccinia virus strain Copenhagen, infection with the thymidine-deleted chimeric poxvirus POXSTG19508 induced syncytia formation with large cells generate by the fusion of membranes from neighboring cells (FIG. 17). Moreover, the cell density indicated that the cell viability following POXSTG19508 infection was more reduced than that following VVTG17111 (FIG. 17), and even more with post-infection time (60h compared to 40h post-infection).

### Chimeric Virus Induced a Superior Specific T Cell Response Against Tumor

To assess tumor and virus-specific T cell responses after therapy, mice bearing CT26 tumors were treated intratumorally with vehicle, VVTG17111 or POXSTG19508 and splenocytes were collected on day 6 post injection and stimulated with a tumor associated peptide antigen (AH-1), a VACV-specific peptide (S9L8) or an irrelevant peptide. The results show that the treatment by POXSTG19508 significantly increased the number of reactive T cells against (FIG. 18) AH-1 compared to treatment by VVTG17111. Additionally, the VACV-specific (S9L8) immune response was similar after treatment with VVTG17111 or POXSTG19508 (FIG. 18). Taken together, these results suggest that chimeric poxvirus elicits an anti-tumor T-cell response superior to that induced by Vaccinia virus strain Copenhagen (VVTG17111) without eliciting a greater anti-viral T-cell response.

### Chimeric Virus Shows Resistance to Complement-Mediated Virus Neutralization by Human Serum

To determine the susceptibility of viruses to complement-mediated lysis, TG6002 and POXSTG19508 were incubated with human serum and functional virus titer was assessed by plaque assays. Heat inactivated serum was used as a negative control. The titer of TG6002 decreased to 7% in the presence of human serum, compared with titer in the presence of heat-inactivated serum (FIG. 19). In contrast, the titer of POXSTG19508 only decreased to 48% in the presence of human serum (FIG. 19), indicating for the chimeric poxvirus an improved resistance to complement-mediated virus neutralization.

### BIBLIOGRAPHIC REFERENCES

Beguin et al., 2020, Mol Ther Oncolytics, 19, 57-66
Broyles et al., 1993, Virol. 195:863-5
Buller et al., 1985, Nature, 317 (6040): 813-5
Burton et al., 2019, Mol Ther Oncolytics, 15, 131-139
Caroll et al., 1997, Virology 238:198-211
Carrey et al., 2011, PLOS ONE, 6 (7) e22442
Carrey et al., 2014, Sci Rep 4:6154 doi 10.1038
Chakrabarti et al., 1997, Biotechniques, 23:1094-7
Chaurasiya et al., 2020, Cancer Gene Therapy 27:125-135
Chernajovsky et al., 2006, BMJ, 332 (7534): 170-2
Chu et al., 1997, J. Exp. Med., 186:1623
Dayhoffed, 1981, Suppl., 3:482-9
Erbs et al., 2000, Cancer Res., 60 (14): 3813-22
Erbs et al., 2008, Cancer Gene Ther. 2008, 15, 18-28
Fallaux et al., 1998, Human Gene Ther. 9:1909-17
Fenner and Comben, 1958, Virology, 5, 530-548
Filley et al., 2017, Front Oncol. 7, 106. doi: 10.3389/fonc.2017.00106
Foloppe et al., 2008, Gene Ther., 15, 1361-1371. doi: 10.1038/gt.2008.82
Foloppe et al., 2019, Mol Ther Oncolytics, 14, 1-14
Graham et al., 1997, J. Gen. Virol. 36:59-72
Guse et al., 2011, Expert Opinion Biol. Ther.11 (5): 595-608
Haddad et al., 2017, Front Oncol., 7, 96. doi: 10.3389/fonc.2017.00096
Hammad et al., 2020, Mol. Ther. Oncolytics, vol. 19 p. 278-282
Hammond et al., 1997, J. Virol. Methods, 66:135-8
Heinrich et al., 2017, Onco Targets Ther., 10, 2389-2401. doi: 10.2147/OTT.S126320
Heo et al., 2013 Nat Med., 19, 329-336. doi: 10.1038/nm.3089
Hermiston et al., 2006, Curr. Opin. Mol. Ther., 8 (4): 322-30
Hruby, 1990, Clin. Microbiol. Rev., 3 (2) 153-170
Katoh et al., 2002, Nucleic Acids Res. 30, 3059-3066
Kirn et al., 2008, Cancer Res. 68 (7): 2071-5
Koerber et al., 2006, Nat. Protocols 1 (2) p.701-706
Krabbe et al., 2018, Cancers, 10, 216
Kritsch et al., 2005, J. Chromatogr. Anal. Technol. Biomed. Life Sci., 822:263-70
Kumar and Boyle, 1990, Virology, 179:151-8
Li et al., 2005, Journal of General Virology, 86, 2969-2977
Macedo et al., 2020, Journal for Immuno Therapy of Cancer; 8
McFadden, 2005, Nat Rev Microbiol., 3, 201-213
Mell et al., 2017, Clin Cancer Res., 23, 5696-5702. doi: 10.1158/1078-0432.CCR-16-3232
Needleman et Wunsh. J.Mol., 1970, Biol. 48,443-453
Nurk et al., 2013, J Comput Biol., 20, 714-737. doi: 10.1089/cmb.2013.0084
O'Leary et al., 2018, Mol. Therap. Vol 9:13-21
Pastor-Anglada et al, 2015, Front. Pharmacol., 6 (13): 1-14
Paszkowski et al., 2016, PLOS Pathogens, 12 (8) e1005824
Ribi et al., 1986, Plenum Publ. Corp., 407-419
Ricordel et al., 2017, Molecular Therapy Oncolytics, 7:1-11
Ricordel et al., 2018, Cancers (Basel), 10, doi: 10.3390/cancers10070231
Ricordel et al., 2018, Oncotarget 9, 35891-35906
Smith et al., 1998, Adv Exp Med Biol, 440, 395-414
Smorlesi, 2005, Gene Ther. 12:1324
Spehner et al., 2000, Virology, 273, 9-15, doi: 10.1006/viro.2000.0411
Suader, 2000, J. Am Acad Dermatol. 43: S6
Sumino et al., 1998, J.Virol. 72:4931
Torres-Domingez et al., 2019, Review Expert Opin Biol Ther.; 19 (6): 561-573
Tritel et al., 2003, J. Immunol., 171:2358
Turner et al. 2008, Virology 380, 226-233
Van der Maaden et al., 2012, J. Control release 161:645-55
Zeh et al., 2015, Mol Ther., 23, 202-214. doi: 10.1038/mt.2014.194
Zhang et al., 2007, Cancer Res. 67:10038-46
EP998568
EP17306012.0
EP3562946
EP2018/066668
U.S. Pat. No. 6,054,438
U.S. Pat. No. 8,394,385
U.S. Pat. No. 8,772,023
WO96/16183
WO98/02522
WO98/04727
WO98/37095
WO98/56415
WO01/66137
WO03/053463
WO2005/042728
WO2005/07857
WO2006/108846
WO2007/056847
WO2007/147528
WO2007/147529
WO2008/114021
WO2008/129058
WO2008/138533
WO2009/065546
WO2009/100521
WO2010/130753
WO2010/130756
WO2011/128704
WO2012/001075
WO2013/022764
WO2014/053571
WO2014/063832
WO2018/031694
WO2020011754

1.-55. (canceled)

56. A chimeric poxvirus comprising a nucleic acid sequence having a sequence identity of at least 96.6% with SEQ ID NO:1.

57. The chimeric poxvirus of claim 56, wherein the chimeric poxvirus comprises nucleic acid fragments from Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR), and Modified Vaccinia virus strain Ankara (MVA).

58. The chimeric poxvirus of claim 56, wherein the chimeric poxvirus comprises a glutamic acid in position 151 of the A34R gene.

59. The chimeric poxvirus of claim 56, wherein the chimeric poxvirus comprises:
(a) at least one Rabbitpox virus strain Utrecht (RPX)-derived nucleic acid sequence selected from:
i. a nucleic acid sequence consisting of nucleotides 562 to 4701 of SEQ ID NO:2 or a sequence with at least 99% identity with nucleotides 562 to 4701 of SEQ ID NO:2,
ii. a nucleic acid sequence consisting of nucleotides 54042 to 59851 of SEQ ID NO:2 or a sequence with at least 99% identity with nucleotides 54042 to 59851 of SEQ ID NO:2,
iii. a nucleic acid sequence consisting of nucleotides 83610 to 88879 of SEQ ID NO:2 or a sequence with at least 99% identity with nucleotides 83610 to 88879 of SEQ ID NO:2,
iv. a nucleic acid sequence consisting of nucleotides 127290 to 130589 of SEQ ID NO:2 or a sequence with at least 99% identity with nucleotides 127290 to 130589 of SEQ ID NO:2,
v. a nucleic acid sequence consisting of nucleotides 137520 to 154979 of SEQ ID NO:2 comprising a glutamic acid in position 151 or a sequence with at least 99% identity with nucleotides 137520 to 154979 of SEQ ID NO:2 comprising a glutamic acid in position 151, and
vi. a nucleic acid sequence consisting of nucleotides 157002 to 162091 of SEQ ID NO:2 or a sequence with at least 99% identity with nucleotides 157002 to 162091 of SEQ ID NO:2;
(b) at least one Cowpox virus strain Brighton (CPX)-derived nucleic acid sequence selected from:
i. a nucleic acid sequence consisting of nucleotides 14242 to 51241 of SEQ ID NO:3 or a sequence with at least 99% identity with nucleotides 14242 to 51241 of SEQ ID NO:3, and
ii. a nucleic acid sequence consisting of nucleotides 59852 to 72141 of SEQ ID NO:3 or a sequence with at least 99% identity with nucleotides 59852 to 72141 of SEQ ID NO:3;
(c) at least one Copenhagen (COP)-derived nucleic acid sequence selected from:
i. a nucleic acid sequence consisting of nucleotides 7612 to 8521 of SEQ ID NO:4 or a sequence with at least 99% identity with nucleotides 7612 to 8521 of SEQ ID NO:4;
(d) at least one Wyeth (WY)-derived nucleic acid sequence selected from:
i. a nucleic acid sequence consisting of 76630 to 78639 of SEQ ID NO:5 or a sequence with at least 99% identity with nucleotides 76630 to 78639 of SEQ ID NO:5,
ii. a nucleic acid sequence consisting of 81060 to 83529 of SEQ ID NO:5 or a sequence with at least 99% identity with nucleotides 81060 to 83529 of SEQ ID NO:5,
iii. a nucleic acid sequence consisting of 116250 to 118459 of SEQ ID NO:5 or a sequence with at least 99% identity with nucleotides 116250 to 118459 of SEQ ID NO:5,
iv. a nucleic acid sequence consisting of 162290 to 164599 of SEQ ID NO:5 or a sequence with at least 99% identity with nucleotides 162290 to 164599 of SEQ ID NO:5,
v. a nucleic acid sequence consisting of 176100 to 179909 of SEQ ID NO:5 or a sequence with at least 99% identity with nucleotides 176100 to 179909 of SEQ ID NO:5, and
vi. a nucleic acid sequence consisting of 181920 to 184099 of SEQ ID NO:5 or a sequence with at least 99% identity with nucleotides 181920 to 184099 of SEQ ID NO:5;
(e) at least one Western Reserve (WR)-derived nucleic acid sequence selected from:
i. a nucleic acid sequence consisting of 169190 to 171579 of SEQ ID NO:6 or a sequence with at least 99% identity with nucleotides 169190 to 171579 of SEQ ID NO:6, and
ii. a nucleic acid sequence consisting of 173730 to 176099 of SEQ ID NO:6 or a sequence with at least 99% identity with nucleotides 173730 to 176099 of SEQ ID NO:6;
(f) at least one Modified Vaccinia Virus Ankara (MVA)-derived nucleic acid sequence selected from:
i. a nucleic acid sequence consisting of 88880 to 90899 of SEQ ID NO:7 or a sequence with at least 99% identity with nucleotides 88880 to 90899 of SEQ ID NO:7,
ii. a nucleic acid sequence consisting of 91460 to 93839 of SEQ ID NO:7 or a sequence with at least 99% identity with nucleotides 91460 to 93839 of SEQ ID NO:7,
iii. a nucleic acid sequence consisting of 95530 to 116249 of SEQ ID NO:7 or a sequence with at least 99% identity with nucleotides 95530 to 116249 of SEQ ID NO:7,
iv. a nucleic acid sequence consisting of 118460 to 127289 of SEQ ID NO:7 or a sequence with at least 99% identity with nucleotides 118460 to 127289 of SEQ ID NO:7, and
v. a nucleic acid sequence consisting of 134800 to 137519 of SEQ ID NO:7 or a sequence with at least 99% identity with nucleotides 134800 to 137519 of SEQ ID NO:7;
or
(g) any combination of (a) to (f).

60. The chimeric poxvirus of claim 56, wherein:
(a) for at least one tumor, the oncolytic power of the chimeric poxvirus is higher than the oncolytic power measured in the same conditions and at the same time post-infection of at least one of the five wild-type parental oncolytic poxvirus strains RPX, CPX, COP, WY, and WR, wherein for a given tumor, a given virus, given conditions, and a given time post-infection, the oncolytic power OP(tumor, virus, conditions, time post-infection) is defined as:
OP(tumor, virus, conditions, time post-infection)=(100−percentage of surviving tumor cells after infection by virus);
(b) for at least one healthy cell, the viral replication in the healthy cell of the chimeric poxvirus is lower than that of at least one of the five oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY), and Vaccinia virus strain Western Reserve (WR);
(c) for at least one organ, the therapeutic index of the chimeric poxvirus is higher than the therapeutic index measured in the same conditions and at the same time post-infection of at least one of the five oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY), and Vaccinia virus strain Western Reserve (WR), wherein for a given organ, a given tumor, a given virus, given conditions and a given time post-infection, an organ-specific therapeutic index TI (organ, tumor, virus, conditions, time post-infection) is defined as:
TI(organ, tumor, virus, conditions, time post-infection)=(replication of virus in organ tumor cells/replication of virus in organ healthy cells);
(d) for at least one producer cell, the extracellular-enveloped virus (EEV)-secretion capacity (SC) (EEV-SC) of the chimeric poxvirus is higher than the EEV-SC measured in the same conditions and at the same time post-infection of at least one of the six parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR), and Modified Vaccinia virus strain Ankara (MVA), wherein, for a given virus, a given producer cell, given conditions and a given time post-infection, an EEV-SC (virus, producer cell, conditions, time post-infection) is defined as:
EEV-SC (virus, producer cell, conditions, time post-infection)=number of EEV particles/number of (EEV+IMV) particles;
(e) for at least one producer cell, the extracellular-enveloped virus (EEV)-secretion capacity (SC) (EEV-SC) of the chimeric poxvirus is higher than the EEV-SC measured in the same conditions and at the same time post-infection of the wild-type Vaccinia virus strain IHD-J;
(f) for at least one tumor, the spreading capacity of the chimeric poxvirus is higher than the spreading capacity measured in the same conditions and at the same time post-infection of at least one of the six parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR), and Modified Vaccinia virus strain Ankara (MVA);
(g) for at least one poxvirus-specific antibody and tumor, the neutralization rate of the chimeric poxvirus is lower than the neutralization rate measured in the same conditions and at the same time post-infection of at least one of the six parental poxvirus strains (optionally variant and/or recombinant) Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR), and Modified Vaccinia virus strain Ankara (MVA), wherein, for a given virus, a given tumor, a given poxvirus-specific antibody, given conditions and a given time post-infection, the neutralization rate (NT(virus, tumor, conditions, time post-infection)) is defined as:
NT(virus, tumor, poxvirus-specific antibody, conditions, time post-infection)=EC50 (with poxvirus-specific antibody)/EC50 (without poxvirus-specific antibody); or
(h) any combination of (a) to (g).

61. The chimeric poxvirus of claim 60, wherein:
(i) the oncolytic power in measured in at least one tumor cell selected from A549, MIA Paca-2, U-87-MG, B16F10, and HepG2 tumor cell lines;
(ii) the replication in at least one healthy cell is measured in at least one healthy cell selected from skin cells and hepatocytes;
(iii) the therapeutic index is measured in liver, organ healthy cells are healthy (preferably primary) hepatocytes and organ tumor cells are HepG2 tumor cells;
(iv) the extracellular-enveloped virus secretion capacity (EEV-SC) is measured in a producer cell that is is A549 tumor cell line; or
(v) any combination of (i) to (iv).

62. The chimeric poxvirus of claim 56, which has been or may be obtained by a method of directed evolution, wherein the method comprises:
(i) infecting a first tumor cell line with a plurality of parental poxvirus strains, wherein the tumor cell line is permissive for each parental poxvirus strain, so as to obtain a first infected tumor cell line;
(ii) amplifying the parental poxvirus strains on the first infected tumor cell line of step (i) during at least 12 h and at most 3 days, so as to obtain one or more distinct chimeric poxviruses through homologous genomic recombination between at least two of the parental poxvirus strains in supernatant;
(iii) collecting the supernatant at the end of step (ii) containing the one or more distinct chimeric poxvirus(es);
(iv) infecting a second tumor cell line with the one or more distinct chimeric poxvirus(es) of the supernatant of step (iii), wherein the second tumor cell line is permissive for each parental poxvirus strain of steps (i) and (ii), so as to obtain a second infected tumor cell line;
(vₐ) amplifying the one or more distinct chimeric poxvirus(es) of step (iv) on the second infected tumor cell line of step (iv) during preferably at least 12 h and at most 24 h and then collecting the supernatant;
(vi) selecting one or more distinct chimeric poxvirus(es) of step (vₐ) having, for at least one third tumor cell line, an oncolytic power higher than the oncolytic power measured in the same conditions and at the same time post-infection of at least one parental oncolytic poxvirus strain in the first tumor cell line of step (i) and/or in the second tumor cell line of step (iv), wherein for a given tumor, a given virus, given conditions and a given time post-infection, an oncolytic power OP(tumor, virus, conditions, time post-infection) is defined as:
OP(tumor, virus, conditions, time post-infection)=(100-percentage of surviving tumor cells after infection by virus).

63. The chimeric poxvirus of claim 62, wherein:
(a) the parental poxviruses strains used in first step (i) are selected in the group consisting of Rabbitpox virus strain Utrecht (RPX), Cowpox virus strain Brighton (CPX), Vaccinia virus strain Copenhagen (COP), Vaccinia virus strain Western Reserve (WR), Vaccinia virus strain Wyeth (WY), Modified Vaccinia virus strain Ankara (MVA), Raccoonpox virus strain Herman (RCN), ORF virus strain NZ2 (ORF), Pseudocowpox strain TJS (PCP), Bovine Papular stomatitis virus strain Illinois 721 (BPS), Myxoma virus strain Lausanne (MYX), Squirrelpox virus strain Kilham (SQF), Fowlpox virus strain FP9 (FPV), Swinepox virus strain Kasza (SPV), Yaba-like disease virus strain Davis (YLD), and Cotia virus strain SP An 232 (CTV);
(b) the permissive tumor cell lines of the method of directed evolution are from higher mammal's origins, preferably the permissive tumor cell lines are selected in the group consisting of A549, CAL-33, HepG2, HCT116, Hela, SK-MEL-1, PANC-1, Hs746T, SK-OV-3, and CV-1; and
(c) both (a) and (b).

64. A variant chimeric poxvirus, consisting of a chimeric poxvirus of claim 56 that has been modified by altering one or more poxviral gene(s).

65. The variant chimeric poxvirus of claim 64, wherein the variant chimeric poxvirus is defective in the J2R locus.

66. The variant chimeric poxvirus of claim 65, wherein the variant chimeric poxvirus comprises a nucleic acid sequence having a sequence identity of at least 99% with SEQ ID NO:8.

67. The variant chimeric poxvirus of claim 65, wherein:
(a) for at least one tumor, the oncolytic power of the variant chimeric poxvirus is higher than the oncolytic power measured in the same conditions and at the same time post-infection of at least one of the five variant parental oncolytic poxvirus strains RPX, CPX, COP, WY, and WR defective in the J2R locus, wherein for a given tumor, a given virus, given conditions, and a given time post-infection, the oncolytic power OP(tumor, virus, conditions, time post-infection) is defined as:
OP(tumor, virus, conditions, time post-infection)=(100-percentage of surviving tumor cells after infection by virus);
(b) for at least one healthy cell, the viral replication in the healthy cell of the variant chimeric poxvirus is lower than that of at least one of the five variant oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY), and Vaccinia virus strain Western Reserve (WR) defective in the J2R locus;
(c) for at least one organ, the therapeutic index of the chimeric poxvirus defective in the J2R locus is higher than the therapeutic index measured in the same conditions and at the same time post-infection of at least one of the five variant oncolytic parental poxvirus strains Rabbitpox virus strain Utrecht (RPX), Cowpox virus strain Brighton (CPX), Vaccinia virus strain Copenhagen (COP), Vaccinia virus strain Wyeth (WY), and Vaccinia virus strain Western Reserve (WR) defective in the J2R locus, wherein for a given organ, a given tumor, a given virus, given conditions and a given time post-infection, an organ-specific therapeutic index TI(organ, tumor, virus, conditions, time post-infection) is defined as:
TI(organ, tumor, virus, conditions, time post-infection)=(replication of virus in organ tumor cells/replication of virus in organ healthy cells);
(d) for at least one producer cell, the extracellular-enveloped virus (EEV)-secretion capacity (SC) (EEV-SC) of the variant chimeric poxvirus is higher than the EEV-SC measured in the same conditions and at the same time post-infection of at least one of the six variant parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR), and Modified Vaccinia virus strain Ankara (MVA) defective in the J2R, wherein, for a given virus, a given producer cell, given conditions and a given time post-infection, an EEV-SC (virus, producer cell, conditions, time post-infection) is defined as:
EEV-SC (virus, producer cell, conditions, time post-infection)=number of EEV particles/number of (EEV+IMV) particles;
(e) for at least one producer cell, the extracellular-enveloped virus (EEV)-secretion capacity (SC) (EEV-SC) of the variant chimeric poxvirus is higher than the EEV-SC measured in the same conditions and at the same time post-infection of the wild-type Vaccinia virus strain IHD-J;
(f) for at least one tumor, the spreading capacity of the variant chimeric poxvirus is higher than the spreading capacity measured in the same conditions and at the same time post-infection of at least one of the six variant parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR), and Modified Vaccinia virus strain Ankara (MVA) defective in the J2R locus;
(g) for at least one poxvirus-specific antibody and tumor, the neutralization rate of the variant chimeric poxvirus is lower than the neutralization rate measured in the same conditions and at the same time post-infection of at least one of the six variant parental poxvirus strains Rabbitpox virus strain Utrecht (RPX), Cowpox virus strain Brighton (CPX), Vaccinia virus strain Copenhagen (COP), Vaccinia virus strain Wyeth (WY), Vaccinia virus strain Western Reserve (WR), and Modified Vaccinia virus strain Ankara (MVA) defective in the J2R locus, wherein, for a given virus, a given tumor, a given poxvirus-specific antibody, given conditions and a given time post-infection, the neutralization rate (NT(virus, tumor, conditions, time post-infection)) is defined as:
NT(virus, tumor, poxvirus-specific antibody, conditions, time post-infection)=EC50 (with poxvirus-specific antibody)/EC50 (without poxvirus-specific antibody); or
(h) any combination of (a) to (g).

68. The chimeric poxvirus of claim 67, wherein:
(i) the oncolytic power in measured in at least one tumor cell selected from A549, MIA Paca-2, U-87-MG, B16F10, and HepG2 tumor cell lines;
(ii) the replication in at least one healthy cell is measured in at least one healthy cell selected from skin cells and hepatocytes;
(iii) the therapeutic index is measured in liver, organ healthy cells are healthy (preferably primary) hepatocytes and organ tumor cells are HepG2 tumor cells;
(iv) the extracellular-enveloped virus secretion capacity (EEV-SC) is measured in a producer cell that is A549 tumor cell line; or
(v) any combination of (i) to (iv).

69. The variant chimeric poxvirus of claim 65, which is defective in one or both of the 14L and F4L loci.

70. The variant chimeric poxvirus of claim 69, wherein the poxvirus comprises a nucleic acid sequence having a sequence identity of at least 99% of identity with SEQ ID NO:9.

71. The variant chimeric poxvirus of claim 69, which is defective in the M2L locus.

72. A recombinant chimeric poxvirus consisting of the chimeric poxvirus of claim 56, which further comprises one or more heterologous nucleic acid(s) of interest inserted in its genome.

73. The recombinant chimeric poxvirus of claim 72, wherein the one or more nucleic acid of interest is selected between immune checkpoint inhibitors, cytokines, agents that affect the regulation of cell surface receptors, agents that affect angiogenesis, agents that stimulates stem cells to produce granulocytes and/or macrophages.

74. A recombinant variant chimeric poxvirus, consisting of the variant chimeric poxvirus of claim 64, which further comprises one or more heterologous nucleic acid(s) of interest inserted in its genome.

75. The recombinant variant chimeric poxvirus of claim 74, wherein the one or more nucleic acid of interest is selected between immune checkpoint inhibitors, cytokines, agents that affect the regulation of cell surface receptors, agents that affect angiogenesis, agents that stimulates stem cells to produce granulocytes and/or macrophages.

76. A process for producing a chimeric poxvirus, a variant chimeric poxvirus, a recombinant chimeric poxvirus, or a recombinant variant chimeric poxvirus, the process comprising:
(i) infecting a producer cell with:
a. himeric poxvirus according to claim 56,
b. a variant chimeric poxvirus consisting of the chimeric poxvirus of claim 56 that has been modified by altering one or more poxviral gene(s),
c. a recombinant chimeric poxvirus consisting of the chimeric poxvirus of claim 56 further comprising one or more heterologous nucleic acid(s) of interest inserted in its genome; or
d. a recombinant variant chimeric poxvirus consisting of the chimeric poxvirus of claim 56 that has been modified by altering one or more poxviral gene(s) and further comprises one or more heterologous nucleic acid(s) of interest inserted in its genome;
(ii) culturing the infected producer cell under conditions which are appropriate for enabling the chimeric poxvirus, variant chimeric poxvirus, recombinant chimeric poxvirus, or recombinant variant chimeric poxvirus to be produced, and;
(iii) recovering the chimeric poxvirus, variant chimeric poxvirus, recombinant chimeric poxvirus, or recombinant variant chimeric poxvirus from the producer cell culture.

77. An isolated nucleic acid encoding:
(i) the chimeric poxvirus according to claim 56,
(ii) a variant chimeric poxvirus consisting of the chimeric poxvirus of claim 56 that has been modified by altering one or more poxviral gene(s),
(iii) a recombinant chimeric poxvirus consisting of the chimeric poxvirus of claim 56 further comprising one or more heterologous nucleic acid(s) of interest inserted in its genome; or
(iv) a recombinant variant chimeric poxvirus consisting of the chimeric poxvirus of claim 56 that has been modified by altering one or more poxviral gene(s) and further comprises one or more heterologous nucleic acid(s) of interest inserted in its genome.

78. A composition comprising a pharmaceutically acceptable vehicle and:
(i) the chimeric poxvirus according to claim 56,
(ii) a variant chimeric poxvirus consisting of the chimeric poxvirus of claim 56 that has been modified by altering one or more poxviral gene(s),
(iii) a recombinant chimeric poxvirus consisting of the chimeric poxvirus of claim 56 further comprising one or more heterologous nucleic acid(s) of interest inserted in its genome; or
(iv) a recombinant variant chimeric poxvirus consisting of the chimeric poxvirus of claim 56 that has been modified by altering one or more poxviral gene(s) and further comprises one or more heterologous nucleic acid(s) of interest inserted in its genome.

79. The composition of claim 78, wherein:
(i) the composition comprises a dose of chimeric poxvirus, variant chimeric poxvirus, recombinant chimeric poxvirus, or recombinant variant chimeric poxvirus comprised between 10⁵ and 5×10⁹ PFU;
(ii) the chimeric poxvirus, variant chimeric poxvirus, recombinant chimeric poxvirus, or recombinant variant chimeric poxvirus is formulated for parenteral route administration; or
(iii) both (i) and (ii).

80. A method for treating a proliferative disease in a subject in need thereof comprising the administration to the subject of:
(i) the chimeric poxvirus according to claim 56,
(ii) a v ariant chimeric poxvirus consisting of the chimeric poxvirus of claim 56 that has been modified by altering one or more poxviral gene(s),
(iii) a recombinant chimeric poxvirus consisting of the chimeric poxvirus of claim 56 further comprising one or more heterologous nucleic acid(s) of interest inserted in its genome;
(iv) a recombinant variant chimeric poxvirus consisting of the chimeric poxvirus of claim 56 that has been modified by altering one or more poxviral gene(s) and further comprises one or more heterologous nucleic acid(s) of interest inserted in its genome; or
(v) a composition comprising a pharmaceutically acceptable vehicle and:
(a) chimeric poxvirus according to claim 56,
(b) a variant chimeric poxvirus consisting of the chimeric poxvirus of claim 56 that has been modified by altering one or more poxviral gene(s),
(c) a recombinant chimeric poxvirus consisting of the chimeric poxvirus of claim 56 further comprising one or more heterologous nucleic acid(s) of interest inserted in its genome; or
(d) a recombinant variant chimeric poxvirus consisting of the chimeric poxvirus of claim 56 that has been modified by altering one or more poxviral gene(s) and further comprises one or more heterologous nucleic acid(s) of interest inserted in its genome.

81. The method of claim 80, wherein the proliferative disease is selected from cancers.

82. The method of claim 81, wherein the cancer is:
(a) selected from lung cancer, renal cancer, bladder cancer, prostate cancer, breast cancer, colorectal cancer, hepatic cancer, gastric cancer, pancreatic cancer, melanoma, ovarian cancer, and glioblastoma;
(b) refractory or resistant to at least one oncolytic virus-based therapy; or
(c) both (a) and (b).

83. The method of claim 81, further comprising the administration of one or more substances effective in anticancer therapy.
